SEQUENCE LISTING (101 sequences)

SEQ ID NO: 1 - protein, 543 aa, Homo sapiens
MAKGEGAESGSAAGLLPTSILQSTERPAQVKKEPKKKKQQLSVCNKLCYALGGAPYQVTGCALGFFLQIYLLDVAQKDEEVVFCFSSFQVGPFSASIILFVGRAWDAITDPLVGLCISKSPWTCLGRLMPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 2 - DNA, 2168 bp, Homo sapiens
  source 1..2168: mol_type = genomic DNA
agagtgcgttcctcgtctgccagccggcttggctagcgcgcggcggccgtggctaaggctgctacgaagcgagcttgggaggagcagcggcctgcggggcagaggagcatcccgtctaccaggtcccaagcggcgtggcccgcgggtcatggccaaaggagaaggcgccgagagcggctccgcggcggggctgctacccaccagcatcctccaaagcactgaacgcccggcccaggtgaagaaagaaccgaaaaagaagaaacaacagttgtctgtttgcaacaagctttgctatgcacttgggggagccccctaccaggtgacgggctgtgccctgggtttcttccttcagatctacctattggatgtggctcagaaggatgaggaagttgtcttttgcttctcctcattccaggtgggccctttctctgcctccatcatcctgtttgtgggccgagcctgggatgccatcacagaccccctggtgggcctctgcatcagcaaatccccctggacctgcctgggtcgccttatgccctggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgctctcggccactttaaccattcccatctggcagtggttcttgacccggtttggcaagaagacagctgtatatgttgggatctcatcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctagggcccgccacgttgcccgaagccaccatgcagaaggccacagaagggatcaggacctgtctgccggcttgctgagcagctggactgcaggtgctaggaagggaactgaagactcaaggaggtggcccaggacacttgctgtgctcactgtggggccggctgctctgtggcctcctgcctcccctctgcctgcctgtggggccaagccctggggctgccactgtgaatatgccaaggactgatcgggcctagcccggaacactaatgtagaaacctttttttttacagagcctaattaataacttaatgactgtgtacatagcaatgtgtgtgtatgtatatgtctgtgagctattaatgttattaattttcataaaagctggaaagca

SEQ ID NO: 3 - protein, 533 aa, Homo sapiens
MAKGEGAESGSAAGLLPTSILQASERPVQVKKEPKKKQQLSICNKLCYAVGGAPYQLTGCALGFFLQIYLLDVAKVEPLPASIILFVGRAWDAFTDPLVGFCISKSSWTRLGRLMPWIIFSTPLAIIAYFLIWFVPDFPSGTESSHGFLWYLLFYCLFETLVTCFHVPYSALTMFISTEQSERDSATAYRMTVEVLGTVIGTAIQGQIVGQAKAPCLQDQNGSVVVSEVANRTQSTASLKDTQNAYLLAAGIIASIYVLCAFILILGVREQRELYESQQAESMPFFQGLRLVMGHGPYVKLIAGFLFTSAFMLVEGNFALFCTYTLDFRNEFQNLLLAIMLSATFTIPIWQWFLTRFGKKTAVYIGISSAVPFLILVALMERNLIVTYVVAVAAGVSVAAAFLLPWSMLPDVIDDFHLKHPHSPGTEPIFFSFYVFFTKFASGVSLGVSTLSLDFANYQRQGCSQPEQVKFTLKMLVTMAPIILILLGLLLFKLYPIDEEKRRQNKKALQALREEASSSGCSDTDSTELASIL

SEQ ID NO: 4 - protein, 374 aa, Homo sapiens
MVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 5 - protein, 491 aa, Homo sapiens
MAKGEGAESGSAAGLLPTSILQSTERPAQVKIYLLDVAQVGPFSASIILFVGRAWDAITDPLVGLCISKSPWTCLGRLMPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 6 - protein, 528 aa, Homo sapiens
MAKGEGAESGSAAGLLPTSILQSTERPAQKEPKKKKQQLSVCNKLCYALGGAPYQVTGCALGFFLQIYLLDVAQVGPFSASIILFVGRAWDAITDPLVGLCISKSPWTCLGRLMPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 7 - protein, 502 aa, Homo sapiens
MAKGEGAESGSAAGLLPTSILQSTERPAQVKKEPKKKKQQLSVCNKLCYALGGAPYQVTGCALGFFLQIYLLDVAQVGPFSASIILFVGRAWDAITDPLVGLCISKSPWTCLGRLMPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 8 - protein, 415 aa, Homo sapiens
MPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 9 - protein, 530 aa, Homo sapiens
MAKGEGAESGSAAGLLPTSILQSTERPAQVKKEPKKKKQQLSVCNKLCYALGGAPYQVTGCALGFFLQIYLLDVAQVGPFSASIILFVGRAWDAITDPLVGLCISKSPWTCLGRLMPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 10 - DNA, 1869 bp, Homo sapiens
  source 1..1869: mol_type = genomic DNA
agagtgcgttcctcgtctgccagccggcttggctagcgcgcggcggccgtggctaaggctgctacgaagcgagcttgggaggagcagcggcctgcggggcagaggagcatcccgtctaccaggtcccaagcggcgtggcccgcgggtcatggccaaaggagaaggcgccgagagcggctccgcggcggggctgctacccaccagcatcctccaaagcactgaacgcccggcccaggtgaaggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgctctcggccactttaaccattcccatctggcagtggttcttgacccggtttggcaagaagacagctgtatatgttgggatctcatcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctagggcccgccacgttgcccgaagccaccatgcagaaggccacagaagggatcaggacctgtctgccggcttgctgagcagctggactgcaggtgctaggaagggaactgaagactcaaggaggtggcccaggacacttgctgtgctcactgtggggccggctgctctgtggcctcctgcctcccctctgcctgcctgtggggccaagccctggggctgccactgtgaatatgccaaggactgatcgggcctagcccggaacactaatgtagaaacctttttttttacagagcctaattaataacttaatgactgtgtacatagcaatgtgtgtgtatgtatatgtctgtgagctattaatgttattaattttcataaaagctggaaagca

SEQ ID NO: 11 - DNA, 2012 bp, Homo sapiens
  source 1..2012: mol_type = genomic DNA
agagtgcgttcctcgtctgccagccggcttggctagcgcgcggcggccgtggctaaggctgctacgaagcgagcttgggaggagcagcggcctgcggggcagaggagcatcccgtctaccaggtcccaagcggcgtggcccgcgggtcatggccaaaggagaaggcgccgagagcggctccgcggcggggctgctacccaccagcatcctccaaagcactgaacgcccggcccaggtgaagatctacctattggatgtggctcaggtgggccctttctctgcctccatcatcctgtttgtgggccgagcctgggatgccatcacagaccccctggtgggcctctgcatcagcaaatccccctggacctgcctgggtcgccttatgccctggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgctctcggccactttaaccattcccatctggcagtggttcttgacccggtttggcaagaagacagctgtatatgttgggatctcatcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctagggcccgccacgttgcccgaagccaccatgcagaaggccacagaagggatcaggacctgtctgccggcttgctgagcagctggactgcaggtgctaggaagggaactgaagactcaaggaggtggcccaggacacttgctgtgctcactgtggggccggctgctctgtggcctcctgcctcccctctgcctgcctgtggggccaagccctggggctgccactgtgaatatgccaaggactgatcgggcctagcccggaacactaatgtagaaacctttttttttacagagcctaattaataacttaatgactgtgtacatagcaatgtgtgtgtatgtatatgtctgtgagctattaatgttattaattttcataaaagctggaaagca

SEQ ID NO: 12 - DNA, 2123 bp, Homo sapiens
  source 1..2123: mol_type = genomic DNA
agagtgcgttcctcgtctgccagccggcttggctagcgcgcggcggccgtggctaaggctgctacgaagcgagcttgggaggagcagcggcctgcggggcagaggagcatcccgtctaccaggtcccaagcggcgtggcccgcgggtcatggccaaaggagaaggcgccgagagcggctccgcggcggggctgctacccaccagcatcctccaaagcactgaacgcccggcccagaaagaaccgaaaaagaagaaacaacagttgtctgtttgcaacaagctttgctatgcacttgggggagccccctaccaggtgacgggctgtgccctgggtttcttccttcagatctacctattggatgtggctcaggtgggccctttctctgcctccatcatcctgtttgtgggccgagcctgggatgccatcacagaccccctggtgggcctctgcatcagcaaatccccctggacctgcctgggtcgccttatgccctggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgctctcggccactttaaccattcccatctggcagtggttcttgacccggtttggcaagaagacagctgtatatgttgggatctcatcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctagggcccgccacgttgcccgaagccaccatgcagaaggccacagaagggatcaggacctgtctgccggcttgctgagcagctggactgcaggtgctaggaagggaactgaagactcaaggaggtggcccaggacacttgctgtgctcactgtggggccggctgctctgtggcctcctgcctcccctctgcctgcctgtggggccaagccctggggctgccactgtgaatatgccaaggactgatcgggcctagcccggaacactaatgtagaaacctttttttttacagagcctaattaataacttaatgactgtgtacatagcaatgtgtgtgtatgtatatgtctgtgagctattaatgttattaattttcataaaagctggaaagca

SEQ ID NO: 13 - DNA, 2045 bp, Homo sapiens
  source 1..2045: mol_type = genomic DNA
agagtgcgttcctcgtctgccagccggcttggctagcgcgcggcggccgtggctaaggctgctacgaagcgagcttgggaggagcagcggcctgcggggcagaggagcatcccgtctaccaggtcccaagcggcgtggcccgcgggtcatggccaaaggagaaggcgccgagagcggctccgcggcggggctgctacccaccagcatcctccaaagcactgaacgcccggcccaggtgaagaaagaaccgaaaaagaagaaacaacagttgtctgtttgcaacaagctttgctatgcacttgggggagccccctaccaggtgacgggctgtgccctgggtttcttccttcagatctacctattggatgtggctcaggtgggccctttctctgcctccatcatcctgtttgtgggccgagcctgggatgccatcacagaccccctggtgggcctctgcatcagcaaatccccctggacctgcctgggtcgccttatgccctggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgtcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctagggcccgccacgttgcccgaagccaccatgcagaaggccacagaagggatcaggacctgtctgccggcttgctgagcagctggactgcaggtgctaggaagggaactgaagactcaaggaggtggcccaggacacttgctgtgctcactgtggggccggctgctctgtggcctcctgcctcccctctgcctgcctgtggggccaagccctggggctgccactgtgaatatgccaaggactgatcgggcctagcccggaacactaatgtagaaacctttttttttacagagcctaattaataacttaatgactgtgtacatagcaatgtgtgtgtatgtatatgtctgtgagctattaatgttattaattttcataaaagctggaaagca

SEQ ID NO: 14 - DNA, 2125 bp, Homo sapiens
  source 1..2125: mol_type = genomic DNA
agagtgcgttcctcgtctgccagccggcttggctagcgcgcggcggccgtggctaaggctgctacgaagcgagcttgggaggagcagcggcctgcggggcagaggagcatcccgtctaccaggtcccaagcggcgtggcccgcgggtcatggccaaaggagaaggcgccgagagcggctccgcggcggggctgctacccaccagcatcctccaaagcactgaacgcccggcccaggtgaagaaccgaaaaagaagaaacaacagttgtctgtttgcaacaagctttgctatgcacttgggggagccccctaccaggtgacgggctgtgccctgggtttcttccttcagatctacctattggatgtggctcaggtgggccctttctctgcctccatcatcctgtttgtgggccgagcctgggatgccatcacagaccccctggtgggcctctgcatcagcaaatccccctggacctgcctgggtcgccttatgccctggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgctctcggccactttaaccattcccatctggcagtggttcttgacccggtttggcaagaagacagctgtatatgttgggatctcatcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctagggcccgccacgttgcccgaagccaccatgcagaaggccacagaagggatcaggacctgtctgccggcttgctgagcagctggactgcaggtgctaggaagggaactgaagactcaaggaggtggcccaggacacttgctgtgctcactgtggggccggctgctctgtggcctcctgcctcccctctgcctgcctgtggggccaagccctggggctgccactgtgaatatgccaaggactgatcgggcctagcccggaacactaatgtagaaacctttttttttacagagcctaattaataacttaatgactgtgtacatagcaatgtgtgtgtatgtatatgtctgtgagctattaatgttattaattttcataaaagctggaaagca

SEQ ID NO: 15 - DNA, 2129 bp, Homo sapiens
  source 1..2129: mol_type = genomic DNA
agagtgcgttcctcgtctgccagccggcttggctagcgcgcggcggccgtggctaaggctgctacgaagcgagcttgggaggagcagcggcctgcggggcagaggagcatcccgtctaccaggtcccaagcggcgtggcccgcgggtcatggccaaaggagaaggcgccgagagcggctccgcggcggggctgctacccaccagcatcctccaaagcactgaacgcccggcccaggtgaagaaagaaccgaaaaagaagaaacaacagttgtctgtttgcaacaagctttgctatgcacttgggggagccccctaccaggtgacgggctgtgccctgggtttcttccttcagatctacctattggatgtggctcaggtgggccctttctctgcctccatcatcctgtttgtgggccgagcctgggatgccatcacagaccccctggtgggcctctgcatcagcaaatccccctggacctgcctgggtcgccttatgccctggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgctctcggccactttaaccattcccatctggcagtggttcttgacccggtttggcaagaagacagctgtatatgttgggatctcatcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctagggcccgccacgttgcccgaagccaccatgcagaaggccacagaagggatcaggacctgtctgccggcttgctgagcagctggactgcaggtgctaggaagggaactgaagactcaaggaggtggcccaggacacttgctgtgctcactgtggggccggctgctctgtggcctcctgcctcccctctgcctgcctgtggggccaagccctggggctgccactgtgaatatgccaaggactgatcgggcctagcccggaacactaatgtagaaacctttttttttacagagcctaattaataacttaatgactgtgtacatagcaatgtgtgtgtatgtatatgtctgtgagctattaatgttattaattttcataaaagctggaaagca

SEQ ID NO: 16 - DNA, 48 bp, synthetic construct
  source 1..48: mol_type = other DNA
gctgagtcaatgcccttctttctcgagaaagaagggcattgactcagc

SEQ ID NO: 17 - DNA, 48 bp, synthetic construct
  source 1..48: mol_type = other DNA
gctcggaacactaatgtagaactcgagttctacattagtgttccgagc

SEQ ID NO: 18 - DNA, 48 bp, synthetic construct
  source 1..48: mol_type = other DNA
gcttacttcctcatctggtttctcgagaaaccagatgaggaagtaagc

SEQ ID NO: 19 - DNA, 48 bp, synthetic construct
  source 1..48: mol_type = other DNA
gcacagcgattcaaggacaaactcgagtttgtccttgaatcgctgtgc

SEQ ID NO: 20 - protein, 7 aa, synthetic construct
NNSTRGG

SEQ ID NO: 21 - protein, 8 aa, synthetic construct
SGARYGYG

SEQ ID NO: 22 - protein, 7 aa, synthetic construct
ANNIRPS

SEQ ID NO: 23 - protein, 10 aa, synthetic construct
GNEDSITYAA

SEQ ID NO: 24 - protein, 10 aa, synthetic construct
GFTFSSYDMA

SEQ ID NO: 25 - protein, 18 aa, synthetic construct
AGITSTGSYTNYGAAVKG

SEQ ID NO: 26 - protein, 19 aa, synthetic construct
SSFGCPYSCWYDIAGSIDA

SEQ ID NO: 27 - protein, 245 aa, synthetic construct
QPSSVSANPGETVKITCSGARYGYGWYQQKSPGSALVTLIYANNIRPSAIPSRFSGSKSGSTATLTITGVRAEDEAVYYCGNEDSITYAAFGAGTTLTVLGGSSRSSGGGGSSGGGGSAVTLDESGGGLQTPKGGLSLVCKASGFTFSSYDMAWVRQAPGKGLEWVAGITSTGSYTNYGAAVKGRATISRDNGQSTVRLQLNSLRAEDTATYFCAKSSFGCPYSCWYDIAGSIDAWGHGTEVIVS

SEQ ID NO: 28 - protein, 103 aa, synthetic construct
ALTQPSSVSANPGETVKITCSGARYGYGWYQQKSPGSALVTLIYANNIRPSAIPSRFSGSKSGSTATLTITGVRAEDEAVYYCGNEDSITYAAFGAGTTLTVL

SEQ ID NO: 29 - protein, 128 aa, synthetic construct
AVTLDESGGGLQTPKGGLSLVCKASGFTFSSYDMAWVRQAPGKGLEWVAGITSTGSYTNYGAAVKGRATISRDNGQSTVRLQLNSLRAEDTATYFCAKSSFGCPYSCWYDIAGSIDAWGHGTEVIVSS

SEQ ID NO: 30-99 - skipped (70 entries)

SEQ ID NO: 100 - protein, 5 aa, synthetic construct
GGGGS

SEQ ID NO: 101 - protein, 6 aa, synthetic construct
HHHHHH

(19) United States
(12) Patent Application Publication
(10) Pub. No.: US 2025/0302918 A1
(43) Pub. Date: Oct. 2, 2025

(54) METHODS AND COMPOSITIONS RELATING TO TREATMENT OF CNS DISEASES

(71) Applicant: PRESIDENT AND FELLOWS OF HARVARD COLLEGE, Cambridge, MA (US)

(72) Inventors: Chenghua GU, Cambridge, MA (US); Ayal BEN-ZVI, Cambridge, MA (US); Benjamin ANDREONE, Cambridge, MA (US)

(73) Assignee: PRESIDENT AND FELLOWS OF HARVARD COLLEGE, Cambridge, MA (US)

(21) Appl. No.: 18/864,132

(22) PCT Filed: May 19, 2023
(86) PCT No.: PCT/US2023/022825

§ 371 Date: Nov. 8, 2024

Related U.S. Application Data
(60) Provisional application No. 63/344,196, filed on May 20, 2022.

(51) Int. Cl. A61K 38/17; A61K 48/00; C07K 16/28; C12N 15/113

(52) U.S. Cl. CPC A61K 38/177; A61K 48/0033; C07K 16/28; C12N 15/1138; C12N 2310/14

(57) ABSTRACT

Described herein are methods relating to the treatment of certain diseases with an agonist of Mfsd2A, and the treatment of certain other diseases with an inhibitor of Mfsd2A.

### CROSS-REFERENCE TO RELATED APPLICATIONS

This application claims benefit under 35 U.S.C. 119(e) of U.S. Provisional Application No. 63/344,196, filed May 20, 2022, the contents of which are incorporated herein by reference in their entirety.

### GOVERNMENT SUPPORT

This invention was made with government support under NS064583, NS092473, NS116820, DA048786, and HL153261 awarded by the National Institutes of Health (NIH). The government has certain rights in the invention.

### TECHNICAL FIELD

The technology described herein relates to the treatment of diseases affecting the central nervous system (CNS) or retina, e.g., by modulating the permeability of the blood-brain barrier (BBB) or blood-retina barrier (BRB).

### BACKGROUND

The central nervous system (CNS) functions in a tightly controlled and stable environment. This is maintained by highly specialized blood vessels that physically seal the CNS and control substance influx/efflux, known as the ‘blood brain barrier’ (BBB). Specialized tight junctions between endothelial cells comprising a single layer that lines the CNS capillaries are the physical seal between blood and brain. BBB selectivity is facilitated by an array of endothelial transporters responsible for the supply of nutrients and for the clearance of waste or toxins. In concert with pericytes and astrocytes, the BBB protects the brain from various toxins and pathogens and provides the proper chemical composition for synaptic transmissions. Therefore, proper function of the CNS critically depends on BBB integrity.

BBB breakdown occurs in many neurodegenerative diseases prior to noticeable neuronal abnormalities. On the other hand, the BBB is also a major obstacle for drug delivery to the CNS, as approximately 98% of small molecules and most large molecules/biologics cannot freely pass through the BBB. Therefore, largely unsuccessful attempts have been made, both to “loosen” the BBB for drugs to pass through and to “re-seal” the BBB to treat various CNS disorders.

### SUMMARY

The technology described herein is directed to methods that “tighten” the BBB to promote its integrity and treat neurodegenerative diseases that degrade the BBB, and methods that temporarily “loosen” the BBB in order to permit other drugs to enter the CNS and promoter the efficacy of those drugs in treating CNS diseases. This modulation of the BBB relates to inhibition or agonism of Mfsd2A to selective transcytose drugs.

In one aspect of any of the embodiments, described herein is a method of treating a disease selected from the group consisting of:
a) a primary central nervous cancer or a metastatic disease in the central nervous system;
b) a neuroinflammatory disorder;
c) a neurocognitive disorder;
d) a neurovascular disease; and
e) a retinal disease;
in a subject in need thereof, the method comprising administering to the subject an agonist of Mfsd2A.
   In one aspect of any of the embodiments, described herein is an agonist of Mfsd2A for use in a method of treating a disease selected from the group consisting of:
a) a primary central nervous cancer or a metastatic disease in the central nervous system;
b) a neuroinflammatory disorder;
c) a neurocognitive disorder;
d) a neurovascular disease; and
e) a retinal disease;
   in a subject in need thereof. In some embodiments of any of the aspects,
a) the primary central nervous cancer or metastatic disease in the central nervous system is selected from the group consisting of: glioblastoma, CNS lymphoma, meningioma, metastatic melanoma, metastatic breast cancer, metastatic lung cancer, metastatic renal cell cancer, and metastatic colorectal cancer;
a) the neuroinflammatory disorder is selected from the group consisting of: multiple sclerosis, infectious encephalitis, COVID-19 encephalopathy, sepsis, and CAR-T neurotoxicity (ICANS) and other toxicities from immuno- and cancer therapeutics;
b) the neurocognitive disorder is selected from the group consisting of: Alzheimer's disease, Huntington disease, Lewy-Body Dementia, and frontotemporal dementia (FTD);
c) the neurovascular disease is selected from the group consisting of: stroke, trauma, and cerebral edema; and
d) the retinal disease is selected from a group consisting of: diabetic retinopathy, macular degeneration, macular edema, retinitis pigmentosa, infectious retinitis, and inflammatory retinitis.

In some embodiments of any of the aspects, the disease is glioblastoma. In some embodiments of any of the aspects, the subject is not further administered a chemotherapeutic. In some embodiments of any of the aspects, the subject is not further administered a further therapeutic agent. In some embodiments of any of the aspects, the agonist is a small molecule agonist. In some embodiments of any of the aspects, the agonist is a pharmaceutical, nutraceutical or dietary formulation, or dietary supplement comprising omega-3 fatty acids and/or linolenic acid. In some embodiments of any of the aspects, the agonist is a cholesterol lowering drug. In some embodiments of any of the aspects, the agonist is a Mfsd2A polypeptide or a vector encoding a Mfsd2A polypeptide. In some embodiments of any of the aspects, the vector is a CNS endothelial-targeting AAV. In some embodiments of any of the aspects, the CNS endothelial-targeting AAV is PHP.V1, BI-30, BR1, or a variant thereof. In some embodiments of any of the aspects, the agonist is targeted to endothelial cells.

In one aspect of any of the embodiments, described herein is a method of treating a disease selected from the group consisting of:
a) a primary central nervous cancer or a metastatic disease in the central nervous system;
b) a neuroinflammatory disease;
c) a neurocognitive disorder;
d) a neuropsychiatric condition;
e) a movement disorder;
f) a critical illness;
g) an enzyme deficiency disorder; and
h) a lysosomal storage disorder.

in a subject in need thereof, the method comprising administering to the subject:
i) an inhibitor of Mfsd2A; and
ii) a central nervous system therapeutic agent.

In one aspect of any of the embodiments, described herein is the combination of:
i) an inhibitor of Mfsd2A; and
ii) a central nervous system therapeutic agent;
   for use a method of treating a disease selected from the group consisting of:

a) a primary central nervous cancer or a metastatic disease in the central nervous system;
b) a neuroinflammatory disease;
c) a neurocognitive disorder;
d) a neuropsychiatric condition;
e) a movement disorder;
f) a critical illness;
g) an enzyme deficiency disorder; and
h) a lysosomal storage disorder.
   In some embodiments of any of the aspects:
a) the primary central nervous cancer or metastatic disease in the central nervous system is selected from the group consisting of: glioblastoma, CNS lymphoma, meningioma, metastatic melanoma, metastatic breast cancer, metastatic lung cancer, metastatic renal cell cancer, and metastatic colorectal cancer;
b) the neuroinflammatory disease is selected from the group consisting of: Multiple Sclerosis (MS), neuromyelitis optica (NMO) Spectrum Disorder, autoimmune encephalitis, infectious meningoencephalitis, progressive multifocal leukoencephalopathy (PML), and paraneoplastic encephalitis;
c) the neurocognitive disorder is selected from the group consisting of: Alzheimer's Dementia, Lewy-Body Dementia, Frontotemporal Dementia, Encephalopathy, and Vascular Demential;
d) the neuropsychiatric condition is selected from the group consisting of: Schizophrenia, Major Depressive Disorder, Attention-Deficit/Hyperactivity Disorder, and Bipolar Disorder;
e) the movement disorder is selected from the group consisting of: Parkinson's Disease, Dystonia, ALS, and Tic disorders;
f) the critical illness is selected from the group consisting of: trauma, stroke, seizure disorders such as epilepsy;
g) the enzyme deficiency disorder is selected from a lysosomal storage disorder, CDKL5 deficiency disorder, X-linked severe paediatric monogenic developmental and epilepsy disorder occurring via a loss of function mutation; and
h) the lysosomal storage disorder is selected from the group consisting of: Tay-Sachs disease, Pompe disease, Gaucher disease, Neimann-Pick disease, Fabry disease, and Mucopolysaccharidoses (MPS) disease.

In some embodiments of any of the aspects, the inhibitor is a small molecule inhibitor. In some embodiments of any of the aspects, the inhibitor is an anti-Mfsd2A antibody reagent, an inhibitory nucleic acid, or a vector encoding an inhibitory nucleic acid. In some embodiments of any of the aspects, the inhibitory nucleic acid comprises or consists of the sequence of one or more of SEQ ID NOs: 16-19. In some embodiments of any of the aspects, the vector is a CNS endothelial-targeting AAV. In some embodiments of any of the aspects, the CNS endothelial-targeting AAV is PHP.V1, BI-30, BR1, or a variant thereof. In some embodiments of any of the aspects, the inhibitor is targeted to endothelial cells. In some embodiments of any of the aspects, the central nervous system therapeutic agent is administered at least 10 days after the inhibitor of Mfsd2A is first administered. In some embodiments of any of the aspects, the central nervous system therapeutic agent is administered at least 14 days after the inhibitor of Mfsd2A is first administered. In some embodiments of any of the aspects, the inhibitor of Mfsd2A is administered for at least 3 months.

### BRIEF DESCRIPTION OF THE DRAWINGS

FIG. 1 depicts a schematic of an inducible Mfsd2A knockout model.

FIG. 2 depicts a workflow for studying the model shown in FIG. 1.

FIG. 3 depicts immunofluorescence images demonstrating the functional impact of acute Mfsd2a ablation on BBB permeability.

FIG. 4 depicts slide scanner immunofluorescence images of medial sagittal sections displaying tracer extravasation across multiple regions of the brain.

FIG. 5 depicts images and graph depicting the amount of AAV9 crossing the BBB in Mfsd2a knockouts.

FIG. 6 depicts a graph of Mfsd2A inhibition, as measured by the uptake of a fluorescent substrate in the presence or absence of candidate inhibitors. Numbers on the X axis identify the compound number. Structures of inhibitors are provided elsewhere herein.

### DETAILED DESCRIPTION

As described herein, the inventors have found improved methods and reagents for modulating Mfsd2A, which controls the integrity of the blood-brain barrier. In some patients, depending on the disease and the clinician's chosen therapeutic approach, Mfsd2A can be agonized to tighten the BBB, which provides its own therapeutic effect by restoring normal, healthy BBB integrity and CNS homeostasis. Alternatively, when delivery of a drug to the CNS is desired, Mfsd2A can be inhibited, providing a temporary loosening of the BBB and permitting the drug to cross the BBB.

A blood-brain barrier (or BBB) is the structure that separates circulating blood from the central nervous system (CNS). The BBB lines the capillaries associated with the CNS and is comprised of endothelial cells and the tight junctions between them. The BBB also includes a basement membrane and astrocytic endfeet. The BBB generally excludes large hydrophilic molecules and bacteria from entering the CNS while allowing the passage of small hydrophobic molecules such as oxygen. Certain molecules are actively transported across the BBB, e.g. glucose.

In some embodiments of any of the aspects, the methods described relate to improving the integrity of the BBB and/or blood-retinal barrier (BRB), e.g., tightening the BBB or BRB. As used herein, “improving the integrity” is an increase in the integrity, impermeability, or condition of the BBB and/or BRB, or a decrease in the rate or level of permeability of the BBB and/or BRB. As compared with an equivalent untreated control, such an improvement is by at least 5%, 10%, 50%, 100%, 200%, 500%, or more as measured by any standard technique. As compared with an equivalent untreated control, such reduction is by at least 5%, 10%, 20%, 40%, 50%, 60%, 80%, 90%, 95%, 99% or more as measured by any standard technique.

In some embodiments of any of the aspects, the methods described relate to decreasing the integrity of the BBB and/or blood-retinal barrier (BRB), e.g., loosening the BBB or BRB. As used herein, “decreasing the integrity” is a decrease in the integrity, impermeability, or condition of the BBB and/or BRB, or a increase in the rate or level of permeability of the BBB and/or BRB. As compared with an equivalent untreated control, such an improvement is by at least 5%, 10%, 50%, 100%, 200%, 500%, or more as measured by any standard technique. As compared with an equivalent untreated control, such reduction is by at least 5%, 10%, 20%, 40%, 50%, 60%, 80%, 90%, 95%, 99% or more as measured by any standard technique.

As used herein, the term “decreasing the permeability” when used in reference to the BBB or the BRB means, at a minimum, that the passage of 10 kD tetramethylrhodamine (TMR)-dextran tracer from blood vessel lumen to tissue parenchyma is reduced by at least 10% in one instance relative to another. A decrease in permeability can be, for example, at least 10%, at least 20%, at least 30%, at least 40%, at least 50%, at least 60%, at least 70%, at least 80%, at least 90% or more, e.g., at least 95%, at least 96%, at least 97%, at least 98%, at least 99% or more. Other tracers, including, e.g., proteins such as horseradish peroxidase or dye conjugates can also be used to measure differences in barrier permeability according to methods known in the art and described herein. In practice, the permeability of a barrier to a given agent or drug can be measured in a manner analogous to the measurements described herein using example tracers. As used herein, the term “increasing the permeability” when used in reference to the BBB or the BRB means, at a minimum, that the passage of 10 kD tetramethylrhodamine (TMR)-dextran tracer from blood vessel lumen to tissue parenchyma is increased by at least 10% in one instance relative to another. An increase in permeability can be, for example, at least 10%, at least 20%, at least 30%, at least 40%, at least 50%, at least 60%, at least 70%, at least 80%, at least 90% or more, e.g., at least 95%, at least 96%, at least 97%, at least 98%, at least 99% or more.

As used herein, “Mfsd2A” or “major facilitator superfamily domain-containing 2A” refers to a transmembrane protein believed to mediate the uptake and transport of tunicamycin. Mfsd2A has a 12 transmembrane alpha-helical domain structure with similarity to the bacterial Na+/melibiose symporters. The sequences of Mfsd2A polypeptides and nucleic acids encoding such polypeptides are known in the art for a number of species, e.g. human Mfsd2A (NCBI Gene ID: 84879 (polypeptide; NCBI Ref Seq: NP_001129965.1 (SEQ ID NO: 1 or 3), NP_001274737.1 (SEQ ID NO: 4), NP_001274738.1 (SEQ ID NO: 5), NP_001336750.1 (SEQ ID NO: 6), NP_001336751.1 (SEQ ID NO: 7), NP_001336752.1 (SEQ ID NO: 8), NP_116182.2 (SEQ ID NO: 9)) (mRNA; NCBI Ref Seq: NM_001136493.3 (SEQ ID NO:2), NM_001287808.2 (SEQ ID NO: 10), NM_001287809.2 (SEQ ID NO: 11), NM_001349821.2 (SEQ ID NO: 12), NM_001349822.2 (SEQ ID NO: 13), NM_001349823.2 (SEQ ID NO: 14), NM_032793.5 (SEQ ID NO: 15)).

A Mfsd2A polypeptide can comprise SEQ ID NO: 1, 3, or 4-9 or a homolog, variant, and/or functional fragment thereof. A nucleic acid encoding a Mfsd2A polypeptide can comprise SEQ ID NO: 2 or 10-15 or a homolog or variant thereof. The polypeptide sequences and nucleic acid sequences encoding any of the other BBB key regulatory genes described herein can readily by obtained by searching the “Gene” Database of the NCBI (available on the World Wide Web at ncbi.nlm.nih.gov/) using the common name or NCBI Gene ID number as the query and selecting the first returned Homo sapiens gene.

In one aspect of any of the embodiments, described herein is a method of treating a disease selected from the group consisting of:
a primary central nervous cancer or a metastatic disease in the central nervous system;
a neuroinflammatory disorder;
a neurocognitive disorder;
a neurovascular disease; and
a retinal disease;
in a subject in need thereof, the method comprising administering to the subject an agonist of Mfsd2A. In some embodiments of any of the aspects,
the primary central nervous cancer or metastatic disease in the central nervous system is selected from the group consisting of: glioblastoma, CNS lymphoma, meningioma, metastatic melanoma, metastatic breast cancer, metastatic lung cancer, metastatic renal cell cancer, and metastatic colorectal cancer;
the neuroinflammatory disorder is selected from the group consisting of: multiple sclerosis, infectious encephalitis, COVID-19 encephalopathy, sepsis, and CAR-T neurotoxicity (ICANS) and other toxicities from immuno- and cancer therapeutics;
the neurocognitive disorder is selected from the group consisting of: Alzheimer's disease, Huntington disease, Lewy-Body Dementia, and frontotemporal dementia (FTD);
the neurovascular disease is selected from the group consisting of: stroke, trauma, and cerebral edema; and
the retinal disease is selected from a group consisting of: diabetic retinopathy, macular degeneration, macular edema, retinitis pigmentosa, infectious retinitis, and inflammatory retinitis.

In some embodiments of any of the aspects, the disease is glioblastoma.

An agonist of Mfsd2A is directly therapeutic, by increasing the integrity (e.g, decreasing the permeability) of the BBB. The effect of a Mfsd2A agonist also means that any other therapeutic is less likely to cross the BBB and enter the CNS. Accordingly, in some embodiments of any of the aspects, the agonist of Mfsd2A is administered as monotherapy. In some embodiments of any of the aspects, the subject administered an agonist of Mfds2A is not administered a further therapeutic agent, e.g., a therapeutic other than the agonist of Mfsd2A. In some embodiments of any of the aspects, the subject administered an agonist of Mfsd2A is not administered a CNS therapeutic agent, e.g., a therapeutic other than the agonist of Mfsd2A. In some embodiments of any of the aspects, the subject administered an agonist of Mfsd2A is not administered a chemotherapeutic. In some embodiments of any of the aspects, the subject administered an agonist of Mfsd2A is further administered a cholesterol lowering drug. Cholesterol lowering drugs can include but are not limited to statins (e.g., atorvastatin, fluvastatin, lovastatin, pravastatin, rosuvastatin, simvastatin, pitavastatin), bile acid sequestrants (e.g., cholestyramine, colestipol, colesevelam), PCSK9 inhibitors (e.g., aliorcumab and evolucumab), ACL inhibitors (e.g., bempedoic acid and ezetimibe), fibrates (e.g., gemfibrozil, fenofibrate, clofibrate), and niacin.

Combinations of the different agonists can be administered, e.g. two or more of: a Mfsd2A polypeptide; a nucleic acid or vector encoding an Mfsd2A polypeptide; a small molecule agonist of Mfsd2A; and/or a pharmaceutical, nutraceutical or dietary formulation, or dietary supplement comprising omega-3 fatty acids and/or linolenic acid. Possible pairwise combinations of Mfsd2A agonists include:
a Mfsd2A polypeptide and a nucleic acid or vector encoding an Mfsd2A polypeptide
a Mfsd2A polypeptide and a small molecule agonist of Mfsd2A;
a Mfsd2A polypeptide and a pharmaceutical, nutraceutical or dietary formulation, or dietary supplement comprising omega-3 fatty acids and/or linolenic acid;
a Mfsd2A polypeptide and a cholesterol lowering drug;
a nucleic acid or vector encoding an Mfsd2A polypeptide and a small molecule agonist of Mfsd2A;
a nucleic acid or vector encoding an Mfsd2A polypeptide and a pharmaceutical, nutraceutical or dietary formulation, or dietary supplement comprising omega-3 fatty acids and/or linolenic acid;
a small molecule agonist of Mfsd2A and a pharmaceutical, nutraceutical or dietary formulation, or a dietary supplement comprising omega-3 fatty acids and/or linolenic acid;
a nucleic acid or vector encoding an Mfsd2A polypeptide and a cholesterol lowering drug;
a nucleic acid or vector encoding an Mfsd2A polypeptide and a cholesterol lowering drug;
a small molecule agonist of Mfsd2A and a cholesterol lowering drug; and
a pharmaceutical, nutraceutical or dietary formulation, or a dietary supplement comprising omega-3 fatty acids and/or linolenic acid and a cholesterol lowering drug.

In some embodiments of any of the aspects, the subject administered a combination of agonists of Mfds2A is not administered a further therapeutic agent, e.g., a therapeutic other than the agonists of Mfsd2A. In some embodiments of any of the aspects, the subject administered a combination of agonists of Mfsd2A is not administered a CNS therapeutic agent, e.g., a therapeutic other than the agonists of Mfsd2A. In some embodiments of any of the aspects, the subject administered a combination of agonists of Mfsd2A is not administered a chemotherapeutic.

As used herein, the term “agonist” refers to an agent which increases the expression and/or activity of the target (e.g., Mfsd2A) by at least 10% or more, e.g. by 10% or more, 50% or more, 100% or more, 200% or more, 500% or more, or 1000% or more. The efficacy of an agonist, e.g. its ability to increase the level and/or activity of the target can be determined, e.g. by measuring the level of an expression product of the target and/or the activity of the target. Methods for measuring the level of a given mRNA and/or polypeptide are known to one of skill in the art, e.g. RTPCR with primers can be used to determine the level of RNA, and Western blotting with an antibody can be used to determine the level of a polypeptide. Suitable primers for a given target are readily identified by one of skill in the art, e.g., using software widely available for this purpose (e.g., Primer3 or PrimerBank, which are both available on the world wide web). Antibodies to polypeptide gene expression products of the immune response regulators described herein are commercially available, e.g., from AbCam (Cambridge, MA). Assays for measuring the activity of the targets described herein are provided elsewhere herein. In some embodiments of any of the aspects, an agonist of Mfsd2A be a Mfsd2A polypeptide, a nucleic acid encoding a Mfsd2A polypeptide, or a small molecule.

Non-limiting examples of agonists of Mfsd2A, can include Mfsd2A polypeptides or variants or functional fragments thereof and nucleic acids encoding Mfsd2A or variants or functional fragments thereof. In some embodiments of any of the aspects, the agonist of Mfsd2A, is a Mfsd2A polypeptide or variants or functional fragment thereof and/or a nucleic acid encoding a Mfsd2A polypeptide or variant or functional fragment thereof. In some embodiments of any of the aspects, the polypeptide agonist can be an engineered and/or recombinant polypeptide. In some embodiments of any of the aspects, the polypeptide agonist can be a nucleic acid encoding a Mfsd2A polypeptide, e.g. a functional fragment thereof. In some embodiments of any of the aspects, the nucleic acid can be comprised by a vector.

In some embodiments of any of the aspects, a polypeptide agonist can comprise one of the sequences described herein for Mfsd2A. In some embodiments of any of the aspects, a polypeptide agonist can consist essentially of one of the sequences provided herein for Mfsd2A. In some embodiments of any of the aspects, a polypeptide agonist can consist of one of the sequences provided herein for Mfsd2A. In some embodiments of any of the aspects, an agonist can comprise a nucleic acid encoding one of the sequences provided herein for Mfsd2A. In some embodiments of any of the aspects, an agonist can be a polypeptide comprising a reference/wild-type sequence described herein with at least 80%, at least 85%, at least 90%, at least 95%, or at least 98% identity to the reference/wild-type Mfsd2A sequence and which retains the activity of the reference/wild-type Mfsd2A sequence. In some embodiments of any of the aspects, an agonist can be a polypeptide comprising a reference/wild-type Mfsd2A sequence described herein with at least 95% identity to the reference/wild-type Mfsd2A sequence and which retains the activity of the reference/wild-type Mfsd2A sequence.

In some embodiments of any of the aspects, the agonist is an exogenous polypeptide. In some embodiments of any of the aspects, the subject is administered exogenous polypeptide, e.g., the polypeptide is produced in vitro and/or synthesized and purified polypeptide is provided to the subject. In some embodiments of any of the aspects, the agonist is an ectopic polypeptide. In some embodiments of any of the aspects, the subject is administered ectopic polypeptide, e.g., the polypeptide is produced in vitro and/or synthesized and purified polypeptide is provided to the subject.

In some embodiments of any of the aspects, the agonist can be a nucleic acid encoding a Mfsd2A polypeptide (or a variant or functional fragment thereof) and/or a vector comprising a nucleic acid encoding a Mfsd2A polypeptide (or a variant or functional fragment thereof). A nucleic acid encoding a Mfsd2A polypeptide can be, e.g., an RNA molecule, a plasmid, and/or an expression vector. In some embodiments of any of the aspects, the nucleic acid encoding a Mfsd2A polypeptide can be an mRNA. In some embodiments of any of the aspects, the nucleic acid encoding a Mfsd2A polypeptide can be a modified mRNA. In some embodiments of any of the aspects, the agonist can be a nucleic acid encoding a Mfsd2A polypeptide, e.g., exogenous and/or ectopic Mfsd2A polypeptide. In some embodiments of any of the aspects, the subject is administered the nucleic acid encoding exogenous and/or ectopic Mfsd2A polypeptide, e.g., the nucleic acid is transcribed and/or translated after the administering step to provide exogenous and/or ectopic Mfsd2A polypeptide to the subject.

In some embodiments of any of the aspects, a polypeptide or nucleic acid as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, the agonist and/or inhibitor is administered as a nucleic acid. In some embodiments of any of the aspects, a nucleic acid encoding the agonist and/or inhibitor is administered. In some embodiments of any of the aspects, the subject is administered a vector comprising a nucleic acid. Vectors can be, e.g., a DNA or RNA vector.

In some embodiments of any of the aspects, the agonist is a vector encoding a Mfsd2A polypeptide. In some embodiments of any of the aspects, the agonist is a vector encoding a Mfsd2A polypeptide, wherein the vector is a CNS endothelial-targeting AAV, e.g., an AAV vector that preferentially binds to and/or enters CNS endothelial cells. Such vectors are known in the art and include PHP.V1, BI-30, BR1, or a variant thereof, which are described elsewhere herein.

In some embodiments of any of the aspects, the agonist of Mfsd2A is a small molecule agonist. Small molecule agonists can be identified using a fluorescence screen for Mfsd2A activity, e.g., a cell-based Mfsd2a lipid transport assay. In such assays, a fluorescent Mfsd2a substrate, TopFluor LPC, is added to cells (e.g., HEK293 cells) lacking Mfsd2a or stably expressing Mfsd2a. Control cells, which do not express Mfsd2a should not take up TopFluor LPC and thus have minimal fluorescent signal. Mfsd2a-expressing cells should take up TopFluor LPC and fluoresce green; TopFluor LPC signal in Mfsd2a cells+agonist should be increased. In exemplary conditions, cells can be incubated with 100 nM TopFluor LPC for 30 minutes, then fixed and imaged with spinning-disk confocal microscopy. Such assays are further described in, e.g., International Patent Application PCT/US2022/012425; Nguyen, L. N. et al. Nature 509, 503-506 (2014); Andreone, B. J. et al. Neuron 94, 581-594 e585 (2017); and Wood et al. Nature 596 (7872): 444-448 (2021); each of which is incorporated by reference herein in its entirety. In some embodiments of any of the aspects, the agonist of Mfsd2A is a small molecule agonist.

In some embodiments of any of the aspects, the agonist of Mfsd2A is a pharmaceutical, nutraceutical or dietary formulation, or dietary supplement comprising omega-3 fatty acids and/or linolenic acid. As used herein, the term “omega-3 fatty acids” includes natural and synthetic omega-3 fatty acids, as well as pharmaceutically acceptable esters, free acids, triglycerides, derivatives, conjugates, precursors, salts, and mixtures thereof. Omega-3 fatty acids can include, but are not limited to, hexadecatrienoic acid (HTA); α-Linolenic acid (ALA); Stearidonic acid (SDA); Eicosatrienoic acid (ETE); Eicosatetraenoic acid (ETA); Eicosapentaenoic acid (EPA); Heneicosapentaenoic acid (HPA); Docosapentaenoic acid (DPA); Clupanodonic acid; Docosahexaenoic acid (DHA); Tetracosapentaenoic acid; and Tetracosahexaenoic acid (Nisinic acid). Omega-3 fatty acids for use in the methods and compostions described herein can have a high content of eicosapentaenoic acid (EPA) as well as docosahexaenoic acid (DHA). The omega-3-fatty acids may be from marine or synthetic origin. For example, a suitable source of omega-3 fatty acids is fish or seal oil. Suitable fish oil sources include deep-sea fish, shark, salmon, cod, salmon, bonito, mackerel, Atlantic mackerel, haddock, herring, mahi mahi, menhaden, mackerel, caplin, tilapia, pacific saury, krill, anchovies, pollock, trout, whitefish, tuna, smelt, shad, and sardines, cold-water fish as described elsewhere herein, and the like.

The fatty acid(s) according to the present disclosure may be derived from animal oils and/or non-animal oils. In some embodiments of the present disclosure, the fatty acid(s) are derived from at least one oil chosen from marine oil, algae oil, plant-based oil, and microbial oil. Marine oils include, for example, fish oil, such as tuna fish oil, krill oil, and lipid composition derived from fish. Plant-based oils include, for example, flaxseed oil, canola oil, mustard seed oil, and soybean oil. Microbial oils include, for example, products by Martek. In at least one embodiment of the present disclosure, the fatty acid(s) are derived from a marine oil, such as a fish oil. In at least one embodiment, the marine oil is a purified fish oil.

Examples of further omega-3 fatty acids and mixtures thereof encompassed by the present disclosure include the omega-3 fatty acids as defined in the European Pharmacopoeia Omega-3 Triglycerides, the European Pharmacopoeia Omega-3 acid Ethyl Esters 60, or the Fish oil rich in omega-3 acids monograph; which are incorporated by reference herein in their entireties.

Commercial examples of omega-3 fatty acids suitable for the present disclosure comprise different fatty acid mixtures (e.g., that can be in the form of triglycerides (TG), ethyl esters (EE), free fatty acid form (FA) and/or as phospholipids) including, but not limited to: Incromega™ omega-3 marine oil concentrates such as Incromega™ E1070, Incromega™ TG7010 SR, Incromega™ E7010 SR, Incromega™ TG6015, Incromega™ EPA500TG SR, Incromega™ E400200 SR, Incromega™ E4010, Incromega™ DHA 700TG SR, Incromega™ DHA700E SR, Incromega™ DHA500TG SR, Incromega™ TG3322 SR, Incromega™ E3322 SR, Incromega™ TG3322, Incromega™ E3322, Incromega™ Trio TG/EE (Croda International PLC, Yorkshire, England); EPAX6000FA, EPAX5000TG, EPAX4510TG, EPAX2050TG, EPAX7010EE, EPAX5500EE, EPAX5500TG, EPAX5000EE, EPAX5000TG, EPAX6000EE, EPAX6000TG, EPAX6000FA, EPAX6500EE, EPAX6500TG, EPAX4510TG, EPAX1050TG, EPAX2050TG, EPAX 7010TG, EPAX7010EE, EPAX6015TG/EE, EPAX4020TG, and EPAX4020EE (EPAX is a wholly-owned subsidiary of Norwegian company Austevoll Seafood ASA); MEG-3® EPA/DHA fish oil concentrates (Ocean Nutrition Canada); DHA FNO “Functional Nutritional Oil” and DHA CL “Clear Liquid” (Lonza); Superba™ Krill Oil (Aker); omega-3 products comprising DHA produced by Martek; Neptune krill oil (Neptune); cod-liver oil products and anti-reflux fish oil concentrate (TG) produced by Møllers; Lysi Omega-3 Fish oil; Seven Seas Triomega® Cod Liver Oil Blend (Seven Seas); and Fri Flyt Omega-3 (Vesterålens).

The fish oil and/or omega-3 fatty acids used herein can be purified, e.g., to meet the quality standards for parenteral administration. In some embodiments of any of the aspects, the fish oil and/or omega-3 fatty acids can be enriched with omega-3 fatty acid triglycerides. Methods of extracting and refining oils are well known in the art.

In some embodiments of any of the aspects described herein, the omega-3 fatty acids and/or fish oil can be highly refined, e.g., highly enriched beyond the initial content of omega-3 fatty acids and their triglycerine compound as part of this specific procedure.

In some embodiments of any of the aspects, the omega-3 fatty acid comprises DHA (Docosahexaenoic acid). In some embodiments of any of the aspects, the omega-3 fatty acid consists of DHA (Docosahexaenoic acid). In some embodiments of any of the aspects, the omega-3 fatty acid is DHA (Docosahexaenoic acid).

The DHA can be in the form of a phospholipid comprising DHA, the phospholipid being selected, for example, from a lysophosphatidylethanolamine (LPE), a phosphatidylcholine (PC), a phosphatidylethanolamine (PE), a lysophosphatidylcholine (LPC), and a phosphatidylserine (PS). Analogs of the noted lipids that promote barrier function can also be used. As used herein, “DHA phospholids” refers to a phospholipid comprising DHA.

In some embodiments of any of the aspects, the omega-3 fatty acid comprises lysophosphatidylcholine (LPC) containing DHA (LPC-DHA). In some embodiments of any of the aspects, the omega-3 fatty acid is lysophosphatidylcholine (LPC) containing DHA (LPC-DHA).

In some embodiments of any of the aspects, the composition administered to the subject can comprise DHA and/or DHA phospholipids. In some embodiments of any of the aspects, the composition administered to the subject can consist essentially of DHA and/or DHA phospholipids. In some embodiments of any of the aspects, the composition administered to the subject can consist of DHA and/or DHA phospholipids.

In some embodiments of any of the aspects, the omega-3 fatty acid comprises or consists of C34:0 PC. In some embodiments of any of the aspects, the omega-3 fatty acid comprises or consists of C38:4 PE. In some embodiments of any of the aspects, the omega-3 fatty acid comprises or consists of C34:0 PC and C38:4 PE. In some embodiments of any of the aspects, the omega-3 fatty acid comprises or consists of DHA, C34:0 PC, and C38:4 PE.

In some embodiments of any of the aspects, the composition, formulation, or supplement comprises sufficient quantities of one or more omega-3 fatty acids to provide in the subject's total daily consumption of nutrients: omega-3 fatty acids as greater than 5%, 10%, 15%, 20%, 25%, 30%, 35%, 40%, 45%, 50%, 55%, 60%, 65%, 70%, 75%, 80%, 85%, 90%, 95%, 98%, 99%, or 100% of the fat in the subject's total daily consumption of nutrients. In some embodiments of any of the aspects, the composition, formulation, or supplement comprises sufficient quantities of linolenic acids to provide in the subject's total daily consumption of nutrients: omega-3 fatty acids as greater than 5%, 10%, 15%, 20%, 25%, 30%, 35%, 40%, 45%, 50%, 55%, 60%, 65%, 70%, 75%, 80%, 85%, 90%, 95%, 98%, 99%, or 100% of the fat in the subject's total daily consumption of nutrients. In some embodiments of any of the aspects, the composition, formulation, or supplement comprises sufficient quantities of one or more omega-3 fatty acids to provide in the subject's circulation: omega-3 fatty acids as greater than 5%, 10%, 15%, 20%, 25%, 30%, 35%, 40%, 45%, 50%, 55%, 60%, 65%, 70%, 75%, 80%, 85%, 90%, 95%, 98%, 99%, or 100% of the circulating total fat in the subject. In some embodiments of any of the aspects, the composition, formulation, or supplement comprises sufficient quantities of linolenic acid to provide in the subject's circulation: linolenic acid as greater than 5%, 10%, 15%, 20%, 25%, 30%, 35%, 40%, 45%, 50%, 55%, 60%, 65%, 70%, 75%, 80%, 85%, 90%, 95%, 98%, 99%, or 100% of the circulating total fat in the subject.

In some embodiments of any of the aspects, the agonist compositions described herein are pharmaceutical, nutraceutical or dietary formulations, and/or dietary supplements. In some embodiments, the agonist compositions described herein, and/or the omega-3 fatty acids described herein are food or pharmaceutical grade.

In some embodiments, a nutraceutical, dietary formulation, and/or dietary supplement can be provided in the form of a shake, meal replacement shake, drink, smoothie, powder, bars, or the like.

As used herein, “nutraceutical” refers to compounds and compositions that are useful in both the nutritional and pharmaceutical field of application. Thus, nutraceutical compositions of the present invention may be used as supplements and/or alternatives to food and beverages, and as pharmaceutical formulations for enteral or parenteral application which may be solid formulations such as capsules or tablets, or liquid formulations, such as solutions or suspensions. In some embodiments of the present invention, nutraceutical compositions may also comprise food and beverages as described herein, as well as supplement compositions, for example dietary supplements.

A dietary formulation can contain all essential amino acids, as well as essential vitamins and minerals to ensure that the recipient is obtaining all necessary nutrients. When the formulation is in the form of a dietary supplement (food oil), the formulation can provide about 5% to 60% of total energy expenditure in terms of calories. Use of such a supplement is expected to produce the same beneficial results as described herein for the dietary formulations when combined with a diet. The dietary formulation or supplement can also be made in powder form by increasing the percent total solids of the formula, using procedures well known to those skilled in the art. The concentrate or powder can then reconstituted for feeding by adding water (tap or deionized-sterilized water).

In some embodiments of any of the aspects, a dietary formulation or supplement can further comprise additional components such as one or more essential fatty acids, a source of carbohydrate, a source of protein, a source of vitamins and minerals, and an emulsifier. The source of carbohydrates can be any simple or complex carbohydrate, e.g., monosaccharides, disaccharides, or oligosaccharides. In one embodiment the source of carbohydrate is at least one of corn starch, dextrose, and glucose. The source of protein can be any protein hydrolysate or peptide mixtures, amino acid mixtures of high biologic values, e.g., meat, milk, egg or soy proteins. The protein hydrolysate can be partially hydrolyzed in nature and include a substantial fraction of variable chain length peptides, e.g., medium or short chain peptides, e.g., di- and tri-peptides, but have less than about 10% free amino acids, more preferably less than about 5% free amino acids. In one embodiment, only the highest biological value proteins are hydrolyzed, e.g., whey, lactalbumin, casein, egg white, egg solids, soy, or delactosed milk solids. In other embodiments, the protein source is lactose-free, and free amino acids are avoided in the formulation. In some embodiments of any of the aspects, a dietary formulation can further comprise a source of vitamins and minerals. For example, vitamins and minerals in accordance with, or approximately, the Recommended Dietary Allowance (RDA), now called the Daily Reference Intake (DRI). The dietary formulations can also contain nutrients not recommended by the DRI, e.g., nucleotides, beta-carotene, carnitine, and taurine. In some embodiments of any of the aspects, a dietary formulation can further comprise an emulsifier or other inactive ingredients such as sweeteners and/or flavorings, which can be artificial. A dietary formulations of the present invention may be in the form of a dietary supplement (food oil) or used in a feeding regimen. In one embodiment, the dietary formulation is provided in a form suitable for oral administration. The dietary formulation can contain all essential amino acids, as well as essential vitamins and minerals to ensure that the recipient is obtaining all necessary nutrients. When the formulation is in the form of a dietary supplement (food oil), the formulation should provide about 5% to 60% of total energy expenditure in terms of calories.

In some embodiments of any of the aspects, the agonist of Mfsd2A can be cholesterol-lowering drug.

The agonist compositions described herein can be administered orally, intravenously, or intraarterially. The compositions and formulations described herein can further include a pharmaceutically or nutritionally acceptable carrier.

The agonist compositions of the invention can be administered in such oral dosage forms as immediate release, controlled release and/or sustained release tablets or capsules, pills, powders, granules, elixers, tinctures, suspensions, syrups and emulsions. For oral administration in the form of a tablet or capsule (e.g., a gelatin capsule), a pharmaceutical-grade omega-3 fatty acid and/or linolenic acid can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or β-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

Examples of formulations suitable for oral administration include, but are not limited to food oil present in, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, health bars, confections, animal feeds, cereals, yogurts, cereal coatings, foods, nutritive foods, functional foods and combinations thereof.

The agonist compositions of the invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is further hydrated with an aqueous solution of a drug to a form lipid layer encapsulating the drug, as described in U.S. Pat. No. 5,262,564.

For solid compositions, excipients include pharmaceutical grades of mannitol, lactose, starch, magnesium stearate, sodium saccharin, talcum, cellulose, glucose, sucrose, magnesium carbonate, and the like may be used. The composition may be also formulated as suppositories using for example, polyalkylene glycols, for example, propylene glycol, as the carrier. In some embodiments, suppositories are advantageously prepared from fatty emulsions or suspensions.

It is also possible for the agonist compositions of the invention to be coupled with soluble polymers as targetable drug carriers. Such polymers can include polyvinylpyrrolidone, pyran copolymer, polyhydroxypropyl-methacrylamide-phenol, polyhydroxyethylaspanamide-phenol, or polyethyleneoxidepolylysine substituted with palmitoyl residues. Furthermore, the compositions of the invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Temporarily loosening the BBB, in combination with the administration of a drug that will act on the CNS, can be therapeutic, by providing more of the drug to the CNS due to the increased permeability of the CNS. In such cases, the therapeutic action of the drug can overcome the costs of loosening the BBB. This similar to the tradeoffs made in surgical treatments (where trauma is created via surgical incisions in order to provide doctors the access necessary to repair internal damage or issues) or chemotherapeutic treatment (where toxicity to the patient is balanced against greater toxicity to the cancer cells). This temporary loosening of the BBB is accomplished by inhibiting Mfsd2a to selectively transcytose desired drugs into the CNS.

Accordingly, in one aspect of any of the embodiments, described herein is a method of treating a disease selected from the group consisting of:
a primary central nervous cancer or a metastatic disease in the central nervous system;
a neuroinflammatory disease;
a neurocognitive disorder;
a neuropsychiatric condition;
a movement disorder;
a critical illness;
an enzyme deficiency disorder; and
a lysosomal storage disorder.
   in a subject in need thereof, the method comprising administering to the subject:
an inhibitor of Mfsd2A; and
a central nervous system therapeutic agent.

In some embodiments of any of any of the aspects:
the primary central nervous cancer or metastatic disease in the central nervous system is selected from the group consisting of: glioblastoma, CNS lymphoma, meningioma, metastatic melanoma, metastatic breast cancer, metastatic lung cancer, metastatic renal cell cancer, and metastatic colorectal cancer; the neuroinflammatory disease is selected from the group consisting of: Multiple Sclerosis (MS), neuromyelitis optica (NMO) Spectrum Disorder, autoimmune encephalitis, infectious meningoencephalitis, progressive multifocal leukoencephalopathy (PML), and paraneoplastic encephalitis;
the neurocognitive disorder is selected from the group consisting of: Alzheimer's Dementia, Lewy-Body Dementia, Frontotemporal Dementia, Encephalopathy, and Vascular Demential;
the neuropsychiatric condition is selected from the group consisting of: Schizophrenia, Major Depressive Disorder, Attention-Deficit/Hyperactivity Disorder, and Bipolar Disorder;
the movement disorder is selected from the group consisting of: Parkinson's Disease, Dystonia, ALS, and Tic disorders;
the critical illness is selected from the group consisting of: trauma, stroke, seizure disorders such as epilepsy;
the enzyme deficiency disorder is selected from a lysosomal storage disorder, CDKL5 deficiency disorder, X-linked severe paediatric monogenic developmental and epilepsy disorder occurring via a loss of function mutation (e.g., usually spontaneous de novo mutations); and
the lysosomal storage disorder is selected from the group consisting of: Tay-Sachs disease, Pompe disease, Gaucher disease, Neimann-Pick disease, Fabry disease, and Mucopolysaccharidoses (MPS) disease.

As described herein, an “inhibitor”, e.g. an inhibitor of Mfsd2A, refers to an agent which can decrease the expression and/or activity of the targeted expression product (e.g. mRNA encoding the target or a target polypeptide), e.g. by at least 10% or more, e.g. by 10% or more, 50% or more, 70% or more, 80% or more, 90% or more, 95% or more, or 98% or more. The efficacy of an inhibitor of, for example, Mfsd2A, e.g. its ability to decrease the level and/or activity of Mfsd2A can be determined, e.g. by measuring the level of an expression product of Mfsd2A and/or the activity of Mfsd2A (e.g. the permeability of the BBB, the measurement of which is described elsewhere herein). Methods for measuring the level of a given mRNA and/or polypeptide are known to one of skill in the art, e.g. RTPCR with primers can be used to determine the level of RNA and Western blotting with an antibody (e.g. an anti-Mfsd2A antibody, e.g. Cat No. ab105399; Abcam; Cambridge, MA) can be used to determine the level of a polypeptide. The activity of, e.g., Mfsd2A can be determined using methods known in the art and described above herein. In some embodiments, the inhibitor of Mfsd2A can be an inhibitory nucleic acid; an aptamer; an antibody reagent; an antibody; or a small molecule. Non-limiting examples of inhibitors of Mfsd2A include inhibitory anti-Mfsd2A antibodies; and inhibitory nucleic acids. In some embodiments of any of the aspects, the Mfsd2A inhibitor is an anti-Mfsd2A antibody reagent. In some embodiments of any of the aspects, the Mfsd2A inhibitor is a small molecule. In some embodiments of any of the aspects, the Mfsd2A inhibitor is an inhibitory nucleic acid. In some embodiments of any of the aspects, the Mfsd2A inhibitor is a vector encoding an inhibitory nucleic acid.

Combinations of the different inhibitors can be administered, e.g. two or more of: an anti-Mfsd2A antibody reagent; a small molecule; an inhibitory nucleic acid; or a vector encoding an inhibitory nucleic acid. Possible pairwise combinations of Mfsd2A inhibitors include:
an anti-Mfsd2A antibody reagent and a small molecule;
an anti-Mfsd2A antibody reagent and an inhibitory nucleic acid;
an anti-Mfsd2A antibody reagent and a vector encoding an inhibitory nucleic acid;
a small molecule and an inhibitory nucleic acid;
a small molecule and a vector encoding an inhibitory nucleic acid; or
an inhibitory nucleic acid and a vector encoding an inhibitory nucleic acid.

In some embodiments, the antibody reagent can bind specifically to Mfsd2A. In some embodiments, the antibody reagent can bind specifically to an extracellular region, domain, and/or epitope of Mfsd2A. In some embodiments, the antibody reagent can bind specifically to an epitope comprising the amino acid corresponding to residue 92 of SEQ ID NO: 3. In some embodiments, the antibody reagent can bind specifically to an epitope comprising the amino acid corresponding to residue 96 of SEQ ID NO: 3.

In some embodiments, the antibody reagent can bind specifically to an epitope comprising amino acids corresponding to residues 1-52 of SEQ ID NO: 3. In some embodiments, the antibody reagent can bind specifically to an epitope comprising at least 4 amino acids of the amino acids corresponding to residues 1-52 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 31-39 of SEQ ID NO: 3. In some embodiments, the antibody reagent can bind specifically to an epitope comprising at least 4 amino acids of the amino acids corresponding to residues 31-39 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 99-114 of SEQ ID NO: 3. In some embodiments, the antibody reagent can bind specifically to an epitope comprising at least 4 amino acids of the amino acids corresponding to residues 99-114 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 175-191 of SEQ ID NO: 3. In some embodiments, the antibody reagent can bind specifically to an epitope comprising at least 4 amino acids of the amino acids corresponding to residues 175-191 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 268-298 of SEQ ID NO: 3. In some embodiments, the antibody reagent can bind specifically to an epitope comprising at least 4 amino acids of the amino acids corresponding to residues 268-298 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 355-360 of SEQ ID NO: 3. In some embodiments, the antibody reagent can bind specifically to an epitope comprising at least 4 amino acids of the amino acids corresponding to residues 355-360 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 406-428 of SEQ ID NO: 3. In some embodiments, the antibody reagent can bind specifically to an epitope comprising at least 4 amino acids of the amino acids corresponding to residues 406-428 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 494-533 of SEQ ID NO: 3. In some embodiments, the antibody reagent can bind specifically to an epitope comprising at least 4 amino acids of the amino acids corresponding to residues 494-533 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 506-509 of SEQ ID NO: 3. In some embodiments, the antibody reagent can bind specifically to an epitope comprising at least 4 amino acids of the amino acids corresponding to residues 506-509 of SEQ ID NO: 3, e.g., 4 amino acids, 5 amino acids, 6 amino acids, 7 amino acids, 8 amino acids, or more amino acids comprised by that region of SEQ ID NO: 3.

In some embodiments, the antibody reagent can bind specifically to an epitope comprising amino acids corresponding to residues 74-77 of SEQ ID NO: 3. In some embodiments, the antibody reagent can bind specifically to an epitope comprising amino acids corresponding to residues 136-150 of SEQ ID NO: 3. In some embodiments, the antibody reagent can bind specifically to an epitope comprising at least 4 amino acids of the amino acids corresponding to residues 136-150 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an eptiope comprising amino acids corresponding to residues 214-246 of SEQ ID NO: 3. In some embodiments, the antibody reagent can bind specifically to an epitope comprising at least 4 amino acids of the amino acids corresponding to residues 214-246 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 319-331 of SEQ ID NO: 3. In some embodiments, the antibody reagent can bind specifically to an epitope comprising at least 4 amino acids of the amino acids corresponding to residues 319-331 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 382-384 of SEQ ID NO: 3. In some embodiments, the antibody reagent can bind specifically to an epitope comprising amino acids corresponding to residues 448-472 of SEQ ID NO: 3. In some embodiments, the antibody reagent can bind specifically to an epitope comprising at least 4 amino acids of the amino acids corresponding to residues 448-472 of SEQ ID NO: 3, e.g., 4 amino acids, 5 amino acids, 6 amino acids, 7 amino acids, 8 amino acids, or more amino acids comprised by that region of SEQ ID NO: 3.

In some embodiments of any of the aspects, the antibody reagent is an antibody reagent as described in International Patent Application PCT/US2022/012425, which is incorporated by reference herein in its entirety; e.g., it comprises at least one heavy or light chain complementarity determining region (CDR) selected from the group consisting of:
(a) a heavy chain CDR1 having the amino acid sequence of SEQ ID NO: 24;
(b) a heavy chain CDR2 having the amino acid sequence of SEQ ID NO: 25;
(c) a heavy chain CDR3 having the amino acid sequence of SEQ ID NO: 26;
(d) a light chain CDR1 having the amino acid sequence of SEQ ID NO: 21;
(e) a light chain CDR2 having the amino acid sequence of SEQ ID NO: 22; and
(f) a light chain CDR3 having the amino acid sequence of SEQ ID NO: 23;
   or a conservative substitution variant of one or more of (a)-(f).

TABLE 1

| | | |
|---|---|---|
| Light Chain CDR1 | SGARYGYG | SEQ ID NO: 21 |
| Light Chain CDR2 | ANNIRPS | SEQ ID NO: 22 |
| Light Chain CDR3 | GNEDSITYAA | SEQ ID NO: 23 |
| Heavy Chain CDR1 | GFTFSSYDMA | SEQ ID NO: 24 |
| Heavy Chain CDR2 | AGITSTGSYTNYGAAVKG | SEQ ID NO: 25 |
| Heavy Chain CDR3 | SSFGCPYSCWYDIAGSIDA | SEQ ID NO: 26 |
| Light Chain Variable | ALTQPSSVSANPGETVKITCSGARYGYGWYQQ | SEQ ID NO: 28 |
| region | KSPGSALVTLIYANNIRPSAIPSRFSGSKSGSTAT | |
| | LTITGVRAEDEAVYYCGNEDSITYAAFGAGTTL | |
| | TVL | |
| Heavy Chain | AVTLDESGGGLQTPKGGLSLVCKASGFTFSSYD | SEQ ID NO: 29 |
| Variable region | MAWVRQAPGKGLEWVAGITSTGSYTNYGAAV | |
| | KGRATISRDNGQSTVRLQLNSLRAEDTATYFC | |
| | AKSSFGCPYSCWYDIAGSIDAWGHGTEVIVSS | |

ScFv variable region Sequence
(SEQ ID NO: 27)
QPSSVSANPGETVKITCSGARYGYGWYQQKSPGSALVTLIYANNIRPS
AIPSRFSGSKSGSTATLTITGVRAEDEAVYYCGNEDSITYAAFGAGTT
LTVLGGSSRSSGGGGSSGGGGSAVTLDESGGGLQTPKGGLSLVCKASG
FTFSSYDMAWVRQAPGKGLEWVAGITSTGSYTNYGAAVKGRATISRDN
GQSTVRLQLNSLRAEDTATYFCAKSSFGCPYSCWYDIAGSIDAWGHGT
EVIVS

In some embodiments of any of the aspects, the antibody reagent comprises heavy chain CDRs having the amino acid sequences of SEQ ID NOs: 24-26 or a conservative substitution variant of such amino acid sequence. In some embodiments of any of the aspects, the antibody reagent comprises heavy chain CDRs having the amino acid sequences of SEQ ID NOs: 24-26.

In some embodiments of any of the aspects, the antibody reagent comprises light chain CDRs having the amino acid sequences of SEQ ID NOs: 21-23 or a conservative substitution variant of such amino acid sequence. In some embodiments of any of the aspects, the antibody reagent comprises light chain CDRs having the amino acid sequences of SEQ ID NOs: 21-23.

In some embodiments of any of the aspects, the antibody reagent comprises heavy chain CDRs having the amino acid sequences of SEQ ID NOs: 24-26 and light chain CDRs having the amino acid sequences of SEQ ID NOs: 21-23 or a conservative substitution variant of such amino acid sequence. In some embodiments of any of the aspects, the antibody reagent comprises heavy chain CDRs having the amino acid sequences of SEQ ID NOs: 24-26 and light chain CDRs having the amino acid sequences of SEQ ID NOs: 21-23.

In some embodiments of any of the aspects, the antibody reagent comprises a heavy chain variable domain comprising SEQ ID NO: 29 or a light chain variable domain comprising SEQ ID NO: 28. In some embodiments of any of the aspects, the antibody reagent comprises a heavy chain variable domain comprising SEQ ID NO: 29 and a light chain variable domain comprising SEQ ID NO: 28. In some embodiments of any of the aspects, the antibody reagent comprises the sequence of SEQ ID NO: 27.

In some embodiments, the inhibitor comprises a bi-specific antibody, e.g. an antibody that can specifically bind to both Mfsd2A and a therapeutic target. The therapeutic target can vary depending upon the disease to be treated. Targets for various diseases of the CNS are known in the art, see, e.g. Corbo and Alsono Adel. Prog Mol Biol Transl Sci 2011 98:47-83 and “Emerging Drugs and Targets for Alzheimer's Disease” Martinez (ed), 2010, RSC Press for discussion of Alzheimer's targets; Hickey and Stacy. Drug Des Devel Thera 2011 5:241-254; Coune et al. Cold Sprin Harb Perspect Med 2012 2: a009431; and Douglas. Expert Review of Neurotherapeutics 2013 13:695-705 for discussion of Parkinson's disease targets. Each of the foregoing references is incorporated by reference in its entirety. In some embodiments of any of the aspects, the subject is in need of treatment for Alzheimer's, and the therapeutic target is beta-secretase 1 or amyloid. For example, amyloid can be targeted using aducanumab.

In some embodiments, the inhibitor of Mfsd2A and the CNS therapeutic agent can be directly conjugated and/or bound to each other, e.g. an antibody-drug conjugate. In some embodiments, binding can be non-covalent, e.g., by hydrogen, electrostatic, or van der waals interactions, however, binding may also be covalent. By “conjugated” is meant the covalent linkage of at least two molecules. In some embodiments, the composition can be an antibody-drug conjugate. In some embodiments, the antibody reagent can be bound to and/or conjugated to multiple therapeutic molecules. In some embodiments, the ratio of a given molecules can be from about 1:1 to about 1,000:1.

In some embodiments of any of the aspects, a bi-specific reagent or antibody drug conjugate can comprise a cleaveable or degradable linker. Such embodiments permit the drug or therapeutic antibody to be released after binding to Mfsd2A, and/or after being endocytosed or transcytosed.

In some embodiments, the linker can be a cleavable linker. For example, the linker comprises a cleavable group. A cleavable group is one which is sufficiently stable under a first set of conditions and can be cleaved to release the two parts the cleavable group is holding together. In a preferred embodiment, the cleavable group is cleaved at least 10 times or more, preferably at least 100 times faster under a first reference condition (which can, e.g., be selected to mimic or represent the blood or serum) than under a second reference condition (which can, e.g., be selected to mimic or represent conditions found in the CNS or intracellular environment).

Cleavable groups are susceptible to cleavage agents, e.g., pH, redox potential or the presence of degradative molecules. Generally, cleavage agents are more prevalent or found at higher levels or activities at the desired site of action of the molecule comprising the cleavable group. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases.

Exemplary cleavable groups include, but are not limited to peptide-based cleavable groups, (e.g., groups that are cleaved by enzymes such as peptidases and proteases, e.g., —NHCHRAC(O)NHCHRBC(O)—, where RA and RB are the R groups of the two adjacent amino acids); redox cleavable groups (e.g., —S—S— and —C(R)2-S—S—, wherein R is H or C1-C6 alkyl and at least one R is C1-C6 alkyl such as CH3 or CH2CH3); phosphate-based cleavable linking groups (e.g., —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(ORk)-S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—, wherein R is optionally substituted linear or branched C1-C10 alkyl); acid cleavable groups (e.g., hydrazones, esters, and esters of amino acids, —C═NN— and —OC(O)—); and ester-based cleavable groups (e.g., —C(O)O—).

In some embodiments, the linker comprises a peptide based cleavable group comprises two or more amino acids.

Inhibitors which bind to Mfsd2A polypeptide (e.g., an antibody reagent) can, after such binding, be endocytosed into the cell expressing Mfsd2A. When the inhibitor is present in a composition and/or conjugated to one or more additional agents, the endocytosis can permit of the additional agent(s) into the cell, i.e. compositions comprising an agent that binds Mfsd2A can be transported across the BBB. In one aspect of any of the embodiments, described herein is a pharmaceutical composition comprising a) an antibody reagent that binds to Mfsd2A; b) a central nervous system therapeutic agent; and c) a pharmaceutically-acceptable carrier.

In some embodiments of any of the aspect, the inhibitor of Mfsd2A is an inhibitory nucleic acid. In some embodiments of any of the aspects, the inhibitory nucleic acid comprises or consists of the sequence of one or more of SEQ ID NOs: 16-19. In some embodiments of any of the aspects, the inhibitory nucleic acid comprises or consists of the sequence of SEQ ID NO: 16. In some embodiments of any of the aspects, the inhibitory nucleic acid comprises or consists of the sequence of SEQ ID NO: 17. In some embodiments of any of the aspects, the inhibitory nucleic acid comprises or consists of the sequence of SEQ ID NO: 18. In some embodiments of any of the aspects, the inhibitory nucleic acid comprises or consists of the sequence of SEQ ID NO: 19.

shRNA#1 (exon 8; SEQ ID NO: 16):
GCTGAGTCAATGCCCTTCTTTCTCGAGAAAGAAGGGCATTGACTCAGC
shRNA#2 (3' UTR; SEQ ID NO: 17):
GCTCGGAACACTAATGTAGAACTCGAGTTCTACATTAGTGTTCCGAGC
shRNA#3 (exon 4; SEQ ID NO: 18):
GCTTACTTCCTCATCTGGTTTCTCGAGAAACCAGATGAGGAAGTAAGC
shRNA#4 (exon 6; SEQ ID NO: 19):
GCACAGCGATTCAAGGACAAACTCGAGTTTGTCCTTGAATCGCTGTGC
Bold: coding strand DNA sequence
Italics: antisense of coding strand sequence

As used herein, “inhibitory nucleic acid” refers to a nucleic acid molecule which can inhibit the expression of a target, e.g., double-stranded RNAs (dsRNAs), inhibitory RNAs (iRNAs), and the like. In some embodiments of any of the aspects, the inhibitory nucleic acid can be a silencing RNA (siRNA), microRNA (miRNA), or short hairpin RNA (shRNA). Inhibitory nucleic acids can also include guide sequence molecules (e.g., a guide RNA) that function, e.g., in combination with an enzyme, to induce insertions, deletions, indels, and/or mutations of a target, thereby inhibiting the expression of the target.

In some embodiments of any of the aspects, an iNA comprises a sequence that is complementary to at least a portion of a target sequence described herein. In some embodiments of any of the aspects, an iNA comprises a sequence at least 15 nucleotides in length that is complementary to at least a portion of a target sequence described herein. In some embodiments of any of the aspects, an iNA comprises a sequence at least 20 nucleotides in length that is complementary to at least a portion of a target sequence described herein.

In some embodiments of any of the aspects, an iNA comprises a sequence that is the reverse complement to at least a portion of a target sequence described herein. In some embodiments of any of the aspects, an iNA comprises a sequence at least 15 nucleotides in length that is the reverse complement to at least a portion of a target sequence described herein. In some embodiments of any of the aspects, an iNA comprises a sequence at least 20 nucleotides in length that is the reverse complement to at least a portion of a target sequence described herein.

In some embodiments of any of the aspects, an iNA comprises a sequence that can specifically hybridize to at least a portion of a target sequence described herein. In some embodiments of any of the aspects, an iNA comprises a sequence at least 15 nucleotides in length that can specifically hybridize to at least a portion of a target sequence described herein. In some embodiments of any of the aspects, an iNA comprises a sequence at least 20 nucleotides in length that can specifically hybridize to at least a portion of a target sequence described herein.

Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA (or modified nucleic acids as described below herein) and which mediates the targeted cleavage of an RNA transcript via an RNA-induced silencing complex (RISC) pathway. In some embodiments of any of the aspects, an iRNA as described herein effects inhibition of the expression and/or activity of a target, e.g. Mfsd2A. In some embodiments of any of the aspects, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA. In some embodiments of any of the aspects, administering an inhibitor (e.g. an iRNA) to a subject results in a decrease in the target mRNA level in the subject by at least about 5%, about 10%, about 20%, about 30%, about 40%, about 50%, about 60%, about 70%, about 80%, about 90%, about 95%, about 99%, up to and including 100% of the target mRNA level found in the subject without the presence of the iRNA.

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target, e.g., it can span one or more intron boundaries. The other strand (the sense strand) includes a region that is complementary to the antisense strand, such that the two strands hybridize and form a duplex structure when combined under suitable conditions. Generally, the duplex structure is between 15 and 30 base pairs in length inclusive, more generally between 18 and 25 base pairs in length inclusive, yet more generally between 19 and 24 base pairs in length inclusive, and most generally between 19 and 21 base pairs in length, inclusive. Similarly, the region of complementarity to the target sequence is between 15 and 30 base pairs in length inclusive, more generally between 18 and 25 base pairs in length inclusive, yet more generally between 19 and 24 base pairs in length inclusive, and most generally between 19 and 21 base pairs in length nucleotides in length, inclusive. In some embodiments of any of the aspects, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

Exemplary embodiments of types of inhibitory nucleic acids can include, e.g. siRNA, shRNA, miRNA, and/or amiRNA, which are well known in the art. One skilled in the art would be able to design further siRNA, shRNA, or miRNA to target the nucleic acid sequence of Mfsd2A (e.g., one or SEQ ID NOs: 2 or 10-15), e.g., using publically available design tools. siRNA, shRNA, or miRNA is commonly made using companies such as Dharmacon (Layfayette, CO) or Sigma Aldrich (St. Louis, MO).

In some embodiments of the various aspects described herein, the inhibitory nucleic acid is a guide nucleic acid (gNA). As used herein, the terms “guide nucleic acid,” “guide sequence,” “crRNA,” “guide RNA,” “single guide RNA,” “gRNA” or “CRISPR guide sequence” refer to a nucleic acid comprising a sequence that determines the specificity of an enzyme, e.g., the Cas DNA binding protein of a CRISPR/Cas system, to a polynucleotide target. The gNA can comprise a polynucleotide sequence with at least partial complementarity with a target nucleic acid sequence, sufficient to hybridize with the target nucleic acid sequence and to direct sequence-specific binding of an enzyme, e.g, a nuclease, to the target nucleic acid sequence.

In some embodiments, the enzyme directed by the gNA is a gene-editing protein, e.g., any nuclease that induces a nick or double-strand break into a desired recognition site. Such enzymes can be native or engineered. These breaks can then be repaired by the cell in one of two ways: non-homologous end joining and homology-directed repair (homologous recombination). In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence can be used as a template for repair of the cleaved target DNA sequence, resulting in the transfer of genetic information from the donor polynucleotide to the target DNA. Therefore, new nucleic acid material may be inserted/copied into the site. The modifications of the target DNA due to NHEJ and/or homology-directed repair can be used for gene correction, gene replacement, gene tagging, transgene insertion, nucleotide deletion, gene disruption, gene mutation, etc.

In one embodiment, the gene-editing protein is a CRISPR-associated nuclease. The native prokaryotic CRISPR-associated nuclease system comprises an array of short repeats with intervening variable sequences of constant length (i.e., clusters of regularly interspaced short palindromic repeats), and CRISPR-associated (“Cas”) nuclease proteins. The RNA of the transcribed CRISPR array is processed by a subset of the Cas proteins into small guide RNAs, which generally have two components as discussed below. There are at least three different systems: Type I, Type II and Type III. The enzymes involved in the processing of the RNA into mature crRNA are different in the 3 systems. In the native prokaryotic system, the guide RNA (“gRNA”) comprises two short, non-coding RNA species referred to as CRISPR RNA (“crRNA”) and trans-acting RNA (“tracrRNA”). In an exemplary system, the gRNA forms a complex with a nuclease, for example, a Cas nuclease. The gRNA: nuclease complex binds a target polynucleotide sequence having a protospacer adjacent motif (“PAM”) and a protospacer, which is a sequence complementary to a portion of the gRNA. The recognition and binding of the target polynucleotide by the gRNA: nuclease complex induces cleavage of the target.

Any CRISPR-associated nuclease can be used in the system and methods of the invention. CRISPR nuclease systems are known to those of skill in the art, e.g. Cas9, Cas12, Cas12a, or the like, see patents/applications U.S. Pat. No. 8,993,233, US 2015/0291965, US 2016/0175462, US 2015/0020223, US 2014/0179770, U.S. Pat. Nos. 8,697,359; 8,771,945; 8,795,965; WO 2015/191693; U.S. Pat. No. 8,889,418; WO 2015/089351; WO 2015/089486; WO 2016/028682; WO 2016/049258; WO 2016/094867; WO 2016/094872; WO 2016/094874; WO 2016/112242; US 2016/0153004; US 2015/0056705; US 2016/0090607; US 2016/0029604; U.S. Pat. Nos. 8,865,406; 8,871,445; each of which are incorporated by reference in their entirety. The nuclease can also be a phage Cas nuclease, e.g., CasΦ (e.g., Pausch et al. Science 369:333-7 (2020); which is incorporated by reference herein in its entirety).

The full-length guide nucleic acid strand can be any length. For example, the guide nucleic acid strand can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments of the various aspects described herein, a nucleic acid strand is less than about 75, 50, 45, 40, 35, 30, 25, 20, 15, 12, or fewer nucleotides in length. For example, the guide nucleic acid sequence is 10-30 nucleotides long.

In addition to a sequence that is complementary to a target nucleic acid, in some embodiments, the gNA also comprises a scaffold sequence. Expression of a gNA encoding both a sequence complementary to a target nucleic acid and scaffold sequence has the dual function of both binding (hybridizing) to the target nucleic acid and recruiting the endonuclease to the target nucleic acid, which may result in site-specific CRISPR activity. In some embodiments, such a chimeric gNA may be referred to as a single guide RNA (sgRNA).

In some embodiments of the various aspects described herein, the guide nucleic acid is designed using a guide design tool (e.g., Benchling™; Broad Institute GPP™; CasOFFinder™; CHOPCHOP™; CRISPOR™; Deskgen™; E-CRISP™; Geneious™; GenHub™; GUIDES™ (e.g., for library design); Horizon Discovery™; IDT™; Off-Spotter™; and Synthego™; which are available on the world wide web).

In some embodiments of any of the aspects, the RNA of an iRNA, e.g., a dsRNA, is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments of any of the aspects, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2-[known as a methylene (methylimino) or MMI backbone], —CH2-O—N(CH3)-CH2-, —CH2-N(CH3)-N(CH3)-CH2- and —N(CH3)-CH2-CH2-[wherein the native phosphodiester backbone is represented as —O—P—O—CH2-].

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33 (1): 439-447; Mook, OR. et al., (2007) Mol Canc Ther 6 (3): 833-843; Grunweller, A. et al., (2003) Nucleic Acids Research 31 (12): 3185-3193).

Modified RNAs can also contain one or more substituted sugar moieties. The iRNAs, e.g., dsRNAs, described herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO]mCH3, O(CH2)·nOCH3, O(CH2)nNH2, O(CH2)nCH3, O(CH2)nONH2, and O(CH2)nON[(CH2)nCH3)]2, where n and m are from 1 to about 10. In some embodiments of any of the aspects, dsRNAs include one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments of any of the aspects, the modification includes a 2′ methoxyethoxy (2′-O—CH2CH2OCH3, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2-N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar.

An inhibitory nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

The preparation of the modified nucleic acids, backbones, and nucleobases described above are well known in the art.

Another modification of an inhibitory nucleic acid featured in the invention involves chemically linking to the inhibitory nucleic acid to one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86:6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In some embodiments of any of the aspects, a small molecule inhibitor of Mfsd2A is a compound having the structural formula I. In some embodiments of any of the aspects, a subject administered an inhibitor of Mfsd2A can be further administered a compound having the structural formula I:

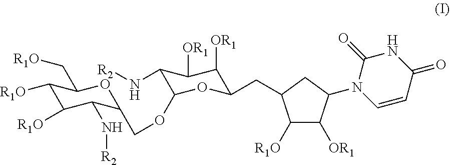

wherein:

each occurrence of R₁ and R₂ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R^{B}; —CO₂R^{B}; -; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl moiety; heteroaryl; hydroxyl; alkoxy; aryloxy; alkylthioxy; arylthioxy; amino; alkylamino; dialkylamino; heteroaryloxy; heteroarylthioxy; or alkylhalo.

In some embodiments, R1 is hydrogen. In some embodiments, at least one R1 is hydrogen. In some embodiments all R1 are hydrogen.

In some embodiments, R1 is a straight chain aliphatic. In some embodiments, R1 is a branched chain aliphatic. In some embodiments, R1 is a straight chain heteroaliphatic. In some embodiments, R1 is a branched chain heteroaliphatic.

In some embodiments, R1 is C1-4 alkyl, C2-4 alkenyl, or C2-4 alkynyl. In some embodiments, R1 is aryl or heteroaryl. In some embodiments R1 is acyl.

In some embodiments, R1 is C1-4 alkyl. In some embodiments, R1 is methyl, ethyl, n-propyl, i-propyl, n-butyl, i-butyl, or t-butyl.

In some embodiments, R1 is —C(O)CH3. In some embodiments, at least one R1 is —C(O)CH3. In some embodiments all R1 are —C(O)CH3.

In some embodiments, R1 is a protecting group.

In some embodiments, R1 is optionally substituted aryl. In some embodiments, R1 is optionally substituted heteroaryl.

In some embodiments, R2 is hydrogen. In some embodiments, at least one R2 is hydrogen. In some embodiments all R2 are hydrogen.

In some embodiments, R2 is a straight chain aliphatic. In some embodiments, R2 is a branched chain aliphatic. In some embodiments, R2 is a straight chain heteroaliphatic. In some embodiments, R2 is a branched chain heteroaliphatic.

In some embodiments, R2 is C1-4 alkyl, C2-4 alkenyl, or C2-4 alkynyl. In some embodiments, R2 is aryl or heteroaryl. In some embodiments R2 is acyl.

In some embodiments, R2 is C1-4 alkyl. In some embodiments, R2 is methyl, ethyl, n-propyl, i-propyl, n-butyl, i-butyl, or t-butyl.

In some embodiments, R2 is —C(O)CH3. In some embodiments, at least one R2 is —C(O)CH3. In some embodiments all R2 are —C(O)CH3.

In some embodiments, R2 is a protecting group.

In some embodiments, R2 is optionally substituted aryl. In some embodiments, R1 is optionally substituted heteroaryl.

In some embodiments, R2 is

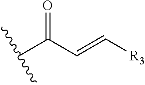

wherein R3 is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl.

In some embodiments, R3 is C1-4 alkyl, C2-4 alkenyl, or C2-4 alkynyl. In some embodiments, R3 is aryl or heteroaryl.

In some embodiments, R3 is C1-4 alkyl. In some embodiments, R2 is methyl, ethyl, n-propyl, i-propyl, n-butyl, i-butyl, or t-butyl. In some embodiments, R3 is i-propyl.

In some embodiments, formula I is tunicamycin:

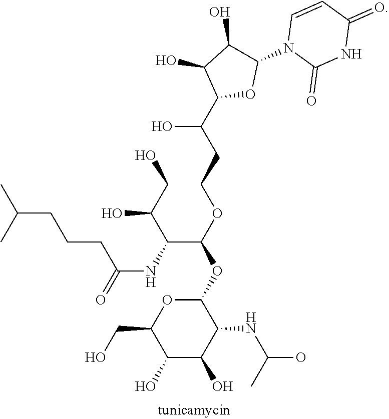

In some embodiments of any of the aspects, a small molecule inhibitor of Mfsd2A is a compound having any one of the following structures:

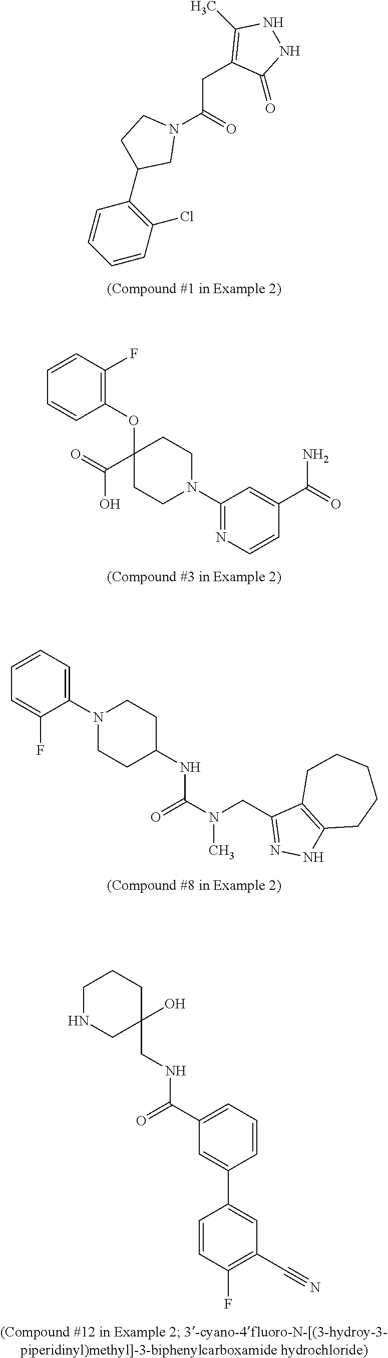 [SMILES: Cc1[nH][nH]c(=O)c1CC(=O)N1CCC(c2ccccc2Cl)C1 ; NC(=O)c1ccnc(N2CCC(Oc3ccccc3F)(C(=O)O)CC2)c1 ; CN(Cc1n[nH]c2c1CCCCC2)C(=O)NC1CCN(c2ccccc2F)CC1 ; N#Cc1cc(-c2cccc(C(=O)NCC3(O)CCCNC3)c2)ccc1F]

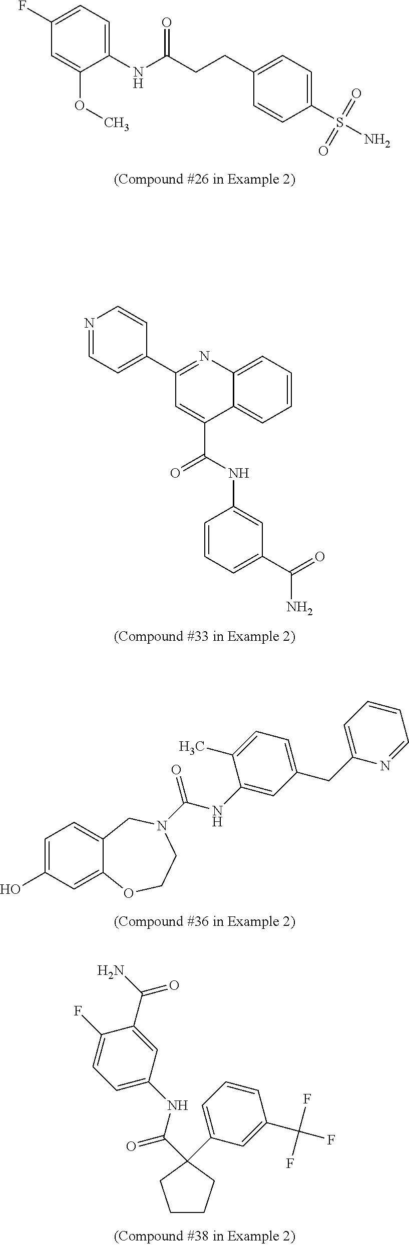 [SMILES: COc1cc(F)ccc1NC(=O)CCc1ccc(S(N)(=O)=O)cc1 ; NC(=O)c1cccc(NC(=O)c2cc(-c3ccncc3)nc3ccccc23)c1 ; Cc1ccc(Cc2ccccn2)cc1NC(=O)N1CCOc2cc(O)ccc2C1 ; NC(=O)c1cc(NC(=O)C2(c3cccc(C(F)(F)F)c3)CCCC2)ccc1F]

A “protecting group” is introduced into a molecule by chemical modification of a functional group. Protecting groups can be but are not limited to alcohol protecting groups (e.g., ester protection, ether protection, ether silyl protection), amine protecting groups (e.g., amine protection, amide protection, carbamate protection, sulfonamide protection), carbonyl protecting groups (e.g., acetal protection, dithiane/dithiolane protection), carboxylic acid protecting groups (e.g., ester protection, ester silyl protection, orthoester protection, oxazoline protection). Examples of ester protection: acetoxy (Ac) and pivolyl (Piv) groups. Examples of ether protections: methyl (Me), methoxymethyl (MOM), methylethoxymethyl (MEM), tetrahydropyranyl (THP), benzyl (Bn), p-methoxybenzyl (PMB), and trityl or triphenylmethane (Tr) groups. Examples of ether silyl protection: trimethylsilyl (TMS), triisopropylsilyl (TIPS), tert-butyldimethylsilyl (TBS or TBDMS) and [2-(trimethylsilyl) ethoxy]methyl (SEM) groups. Examples of amine protection: benzyl (Bn) and p-methoxyphenyl (PMP) groups. Examples of amide protection: acetyl (Ac), trifluoroacetyl (TFA) and Trichloroacetyl groups. Examples of carbamate protection: tert-butyloxycarbonyl (BOC), carbobenzyloxy (Cbz or Z), vinyloxycarbonyl (Voc), allyloxycarbonyl (Alloc), 9-fluorenylmethyloxycarbonyl (Fmoc) groups. Examples of sulfonamide protection: tosyl (Ts) and nosyl (Ns) groups. Examples of acetal protection: dimethyl acetal, 1,3-dioxolanes, 1,3-dioxane. Examples of dithiane/dithiolane protection: 1,3-dithiane, 1,3-dithiolane. One of ordinary skill in the art would know to refer for example to “Protective Groups in Organic Synthesis” Wuts P. G. M. and Greene T. W., editions Wiley-Interscience 4the Edition (Oct. 30, 2006) which is incorporated in entirety by reference.

As used herein, the terms “alkyl,” “alkenyl” and the prefix “alk-” are inclusive of both straight chain and branched chain groups and of cyclic groups, i.e. cycloalkyl and cycloalkenyl. Unless otherwise specified, these groups contain from 1 to 20 carbon atoms, with alkenyl groups containing from 2 to 20 carbon atoms. Preferred groups have a total of up to 10 carbon atoms. Cyclic groups can be monocyclic or polycyclic and preferably have from 3 to 10 ring carbon atoms. Exemplary cyclic groups include cyclopropyl, cyclopentyl, cyclohexyl, cyclopropylmethyl, adamantly, norbornane, and norbornene. This is also true of groups that include the prefix “alkyl-,” such as alkylcarboxylic acid, alkyl alcohol, alkylcarboxylate, alkylaryl, and the like. Examples of suitable alkylcarboxylic acid groups are methylcarboxylic acid, ethylcarboxylic acid, and the like. Examples of suitable alkylacohols are methylalcohol, ethylalcohol, isopropylalcohol, 2-methylpropan-1-ol, and the like. Examples of suitable alkylcarboxylates are methylcarboxylate, ethylcarboxylate, and the like. Examples of suitable alkyl aryl groups are benzyl, phenylpropyl, and the like.

These may be straight chain or branched, saturated or unsaturated aliphatic hydrocarbon, which may be optionally inserted with N, O, or S. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like.

As used herein, the term “alkenyl” means an alkyl, as defined above, containing at least one double bond between adjacent carbon atoms. Alkenyls include both cis and trans isomers. Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like.

As used herein, the term “alkynyl” means any alkyl or alkenyl, as defined above, which additionally contains at least one triple bond between adjacent carbons. Representative straight chain and branched alkynyls include acetylenyl, propynyl, 1-butynyl, 2-butynyl, 1-pentynyl, 2-pentynyl, 3-methyl-1 butynyl, and the like.

The term “aryl” as used herein includes carbocyclic aromatic rings or ring systems. As used herein, the term “aryl” refers to an aromatic 5-8 membered monocyclic, 8-12 membered bicyclic, or 11-14 membered tricyclic ring system. Examples of aryl groups include phenyl, naphthyl, biphenyl, fluorenyl and indenyl.

The term “heteroaryl” includes aromatic rings or ring systems that contain at least one ring hetero atom (e.g., O, S, N). As used herein, the term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, thiazolyl, benzimidazolyl, pyrimidinyl, thiophenyl or thienyl, quinolinyl, indolyl, oxazolyl, isoquinolinyl, isoindolyl, thiazolyl, pyrrolyl, tetrazolyl, pyrazolyl, oxazolyl, thiazolyl, benzofuranyl, benzothiophenyl, carbazolyl, benzoxazolyl, benzimidazolyl, quinoxalinyl, benzothiazolyl, naphthyridinyl, isoxazolyl, isothiazolyl, purinyl, quinazolinyl, and the like.

Heteroaryl rings may also be fused with one or more cyclic hydrocarbon, heterocyclic, aryl, or heteroaryl rings. Heteroaryl includes, but is not limited to, 5-membered heteroaryls having one hetero atom (e.g., thiophenes, pyrroles, furans); 5-membered heteroaryls having two heteroatoms in 1,2 or 1,3 positions (e.g., oxazoles, pyrazoles, imidazoles, thiazoles, purines); 5-membered heteroaryls having three heteroatoms (e.g., triazoles, thiadiazoles); 5-membered heteroaryls having 3 heteroatoms; 6-membered heteroaryls with one heteroatom (e.g., pyridine, quinoline, isoquinoline, phenanthrine, 5,6-cycloheptenopyridine); 6-membered heteroaryls with two heteroatoms (e.g., pyridazines, cinnolines, phthalazines, pyrazines, pyrimidines, quinazolines); 6-membered heretoaryls with three heteroatoms (e.g., 1,3,5-triazine); and 6-membered heteroaryls with four heteroatoms. Particularly preferred heteroaryl groups are 5-10-membered rings with 1-3 heteroatoms selected from O, S, and N.

The aryl, and heteroaryl groups can be unsubstituted or substituted by one or more substituents independently selected from the group consisting of alkyl, alkoxy, methylenedioxy, ethylenedioxy, alkylthio, haloalkyl, haloalkoxy, haloalkylthio, halogen, nitro, hydroxy, mercapto, cyano, carboxy, formyl, aryl, aryloxy, arylthio, arylalkoxy, arylalkylthio, heteroaryl, heteroaryloxy, heteroarylalkoxy, heteroarylalkylthio, amino, alkylamino, dialkylamino, heterocyclyl, heterocycloalkyl, alkylcarbonyl, alkenylcarbonyl, alkoxycarbonyl, haloalkylcarbonyl, haloalkoxycarbonyl, alkylthiocarbonyl, arylcarbonyl, heteroarylcarbonyl, aryloxycarbonyl, heteroaryloxycarbonyl, arylthiocarbonyl, heteroarylthiocarbonyl, alkanoyloxy, alkanoylthio, alkanoylamino, arylcarbonyloxy, arylcarbonythio, alkylaminosulfonyl, alkylsulfonyl, arylsulfonyl, heteroarylsulfonyl, aryldiazinyl, alkylsulfonylamino, arylsulfonylamino, arylalkylsulfonylamino, alkylcarbonylamino, alkenylcarbonylamino, arylcarbonylamino, arylalkylcarbonylamino, arylcarbonylaminoalkyl, heteroarylcarbonylamino, heteroarylalkycarbonylamino, alkylsulfonylamino, alkenylsulfonylamino, arylsulfonylamino, arylalkylsulfonylamino, heteroarylsulfonylamino, heteroarylalkylsulfonylamino, alkylaminocarbonylamino, alkenylaminocarbonylamino, arylaminocarbonylamino, arylalkylaminocarbonylamino, heteroarylaminocarbonylamino, heteroarylalkylaminocarbonylamino and, in the case of heterocyclyl, oxo. If other groups are described as being “substituted” or “optionally substituted,” then those groups can also be substituted by one or more of the above enumerated substituents.

The term “arylalkyl,” as used herein, refers to a group comprising an aryl group attached to the parent molecular moiety through an alkyl group.

As used herein, the term “cyclyl” refers to a nonaromatic 5-8 membered monocyclic, 8-12 membered bicyclic, or 11-14 membered tricyclic ring system, which can be saturated or partially unsaturated. Representative saturated cyclyl groups include, but are not limited to, cyclopropyl, cyclobutyl, cyclopentyl, cyclopentenyl, cyclohexyl, cyclohexenyl, cycloheptyl, cyclooctyl, and the like; while unsaturated cyclyl groups include cyclopentenyl and cyclohexenyl, and the like.

The terms “heterocycle”, “heterocyclyl” and “heterocyclic group” are recognized in the art and refer to nonaromatic 3- to about 14-membered ring structures, such as 3- to about 7-membered rings, whose ring structures include one to four heteroatoms, 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. The heterocycle may include portions which are saturated or unsaturated. In some embodiments, the heterocycle may include two or more rings (e.g., cycloalkyls, cycloalkenyls, cycloalkynyls, aryls and/or heterocyclyls) in which two or more carbons are common to two adjoining rings, e.g., the rings are “fused rings.” In some embodiments, the heterocycle may be a “bridged” ring, where rings are joined through non-adjacent atoms, e.g., three or more atoms are common to both rings. Each of the rings of the heterocycle may be optionally substituted. Examples of heterocyclyl groups include, for example, thiophene, thianthrene, furan, pyran, isobenzofuran, chromene, xanthene, phenoxathin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, dioxane, morpholine, tetrahydrofurane, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with substituents including, for example, halogen, aryl, heteroaryl, alkyl, heteroalkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, CF3, CN, or the like.

As used herein, the term “halogen” refers to iodine, bromine, chlorine, and fluorine.

As used herein, the terms “optionally substituted alkyl,” “optionally substituted cyclyl,” “optionally substituted heterocyclyl,” “optionally substituted aryl,” and “optionally substituted heteroaryl” means that, when substituted, at least one hydrogen atom in said alkyl, cyclyl, heterocyclyl, aryl, or heteroaryl is replaced with a substituent. In the case of an oxo substituent (═O) two hydrogen atoms are replaced. In this regard, substituents include oxo, halogen, alkyl, cyclyl, heterocyclyl, aryl, heteroaryl, —CN, —ORx, —NRxRy, —NRxC(═O)Ry, —NRxSO2Ry, —C(═O)Rx, —C(═O)ORx, —C(═O)NRxRy, —SOnRx and —SOnNRxRy, wherein n is 0, 1 or 2, Rx and Ry are the same or different and independently hydrogen, alkyl, cyclyl, heterocyclyl, aryl or heterocycle, and each of said alkyl, cyclyl, heterocyclyl, aryl and heterocycle substituents may be further substituted with one or more of oxo, halogen, —OH, —CN, alkyl, —ORx, heterocycle, —NRxRy, —NRxC(═O)Ry, —NRxSO2Ry, —C(═O)Rx, —C(═O)ORx, —C(═O)NRxRy, —SOnRx and —SOnNRxRy.

The term “carbonyl,” as used herein, refers to “C(═O)”.

The terms “acyl,” “carboxyl group,” or “carbonyl group” are recognized in the art and can include such moieties as can be represented by the general formula:

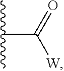

wherein W is ORw, N(Rw)2, SRw, or Rw, Rw being hydrogen, alkyl, heteroalkyl, alkenyl, heteroalkenyl, alkynyl, heteroalkynyl, aryl, heteroaryl, heterocycle, substituted derivatives thereof, or a salt thereof. For example, when W is O-alkyl, the formula represents an “ester,” and when W is OH, the formula represents a “carboxylic acid.” When W is alkyl, the formula represents a “ketone” group, and when W is hydrogen, the formula represents an “aldehyde” group. Those of ordinary skill in the art will understand the use of such terms.

As used herein, the term “substituted” is contemplated to include all permissible substituents of organic compounds, “permissible” being in the context of the chemical rules of valence known to those of ordinary skill in the art. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted phenyl” must still comprise the phenyl moiety and cannot be modified by substitution, in this definition, to become, e.g., a heteroaryl group such as pyridine. In a broad aspect, the permissible substituents include acyclic and cyclic, branched and unbranched, carbocyclic and heterocyclic, aromatic and nonaromatic, fused, and bridged substituents of organic compounds. Illustrative substituents include, for example, those described herein. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms. This invention is not intended to be limited in any manner by the permissible substituents of organic compounds.

A CNS therapeutic agent can be any agent for the treatment of any disease, provided that it is desired that the CNS therapeutic agent reaches the central nervous system. Non-limiting examples of such CNS therapeutic agents can include, antibiotics, antibodies, gabapentin, chemotherapeutics, anti-inflammatories, neurotransmitters, morphines, peptides, polypeptides, nucleic acids (e.g. RNAi-based therapies), psychiatric dugs, and/or therapeutic agents for the treatment of brain cancer; encephalitis; hydrocephalus; Parkinson's disease; neuropathic pain; and a condition treated by the administration of psychiatric drugs. The identity of such CNS therapeutic agents are known in the art and described, e.g. in Ghose et al. J Comb Chem 1999 1:55-68 and Pardridge. NeuroRx 2005 2:3-14; each of which is incorporated by reference herein in its entirety. In some embodiments, a central nervous system therapeutic agent can inhibit the activity and/or expression of a therapeutic target gene associated with a central nervous system disease (e.g. examples of such genes are described elsewhere herein), e.g. it can be an inhibitory nucleic acid or an inhibitory antibody reagent.

Non-limiting examples of CNS therapeutic agents for the conditions described herein include:

| Condition | Exemplary CNS therapeutic agents |
|---|---|
| Glioblastoma | temozolomide, bevacizumab, |
| CNS lymphoma | Acalabrutinib, brentuximab, capanlisib, nealrabine, |
| | axicabtagene ciloleucel, belinostat, bendamustine, |
| | carmustine, bleomycin, boretzomib, brexucabtagene |
| | autoleucel, lisocabtagene maraleucel, zanubrutinib, |
| | chlorambucil, copanlisib, crizotinib, cyclophosphamide, |
| | denileukin difitox, dexamethasone, doxorubicin, |
| | pralatrexate, obinutuzumab, ibritumomab tiuxetan, ibrutinib, |
| | IFNα-2b, romidepsin, pembrolizumab, tisagenelecleucel, |
| | lenalidomide, loncastuximab tesirine, methotrexate, |
| | mogamulizumab, tafassitamab, plerixafor, nelarabine, |
| | obinutuzumab, polatuzumab, pralatrexate, prednisone, |
| | rituximab, selinexor, tazemetostat, bendamustine, umbralisib |
| | tosylate, bortezomib, venetoclax, vinblastine, vorinostat, |
| | zanubrutinib, |
| Meningioma | Interferon, bevacizumab, progesterone, hydroxyurea |
| Metastatic melanoma | Aldesleukin, binimetinib, encorafenib, cobimetinib, |
| | dabrafenib, dacarbazine, talimogene laherparepvec, IFNα-2b, |
| | ipilimumab, pembrolizumab, tebentafusp-tebn, trametinib, |
| | nivolumab, vemuarfenib |
| Metastatic breast cancer | raloxifene, tamoxifen, ademaciclib, paclitaxel, trastuzumab, |
| | everolimus, alpelisib, anastrozole, pamidronate, exemestane, |
| | capecitabine, cyclophosphamide, docetaxel, doxorubicin, |
| | epirubicin, eribulin, fluorouracil, toremifene, fulvestrant, |
| | letrozole, gemcitabine, goserelin, eribulin mesylate, |
| | palbociclib, ixabepilone, pembrolizumab, ribociclib, |
| | lapatinib, olaparib, margetuximab, megestrol, methotrexate, |
| | neratinib, pertuzumab, sacituzumab, talazoparib, taxotere, |
| | atezolizumab, thiotepa, tucatinib, abemaciclib, vinblastine, |
| Metastatic lung cancer | Paclitaxel, afatinib, everolimus, alectinib, pemetrexed, |
| | brigatanib, amivantamab, atezolizumab, bevacizumab, |
| | capmatinib, cemiplimab, ceritinib, crizotinib, ramucirumab, |
| | dabrafenib, dacomitinib, docetaxel, doxorubicin, |
| | durvalumab, entrctinib, erlotinib, mobocertinib, pralsetinib, |
| | gefitinib, gemcitabine, duravalumab, ipilimumab, |
| | pembrozlizumab, lorlatinib, sotorasib, trametinib, |
| | methotrexate, necitumumab, nivolumab, osimertinib, |
| | premetrexed, necitumumab, selpercatinib, entrectinib, |
| | amivantamab, tepotinib, vinorelbine, |
| Metastatic renal cell cancer | Everolimus, aldesleukin, bevacizumab, avelumab, belzutifan, |
| | cabozantinib, tivozanib, axitinib, ipilimumab, |
| | pembrolizumab, lenvatnib, sorafenib, nivolumab, pazopanib, |
| | sunitinib, temsirolimus, |
| Metastatus colorectal cancer | Bevacizumab, irinotecan, capecitabine, cetuximab, |
| | ramucirumab, oxaliplatin, fluorouracil, ipilimumab, |
| | pembrolizumab, leucovorin, trifluridine, tipiracil, nivolumab, |
| | panitumumab, regorafenib, afilbercept, Ziv-afliercept |
| Multiple sclerosis | Ocrelizumab, siponimod, IFNβ-1a, IFNβ-1b, glatiramer, |
| | glatiramer acetate, ofatumumab, teriflunomide, monomethyl |
| | fumarate, dimethyl fumarate, fingolimod, caldribine, |
| | siponimod, ponesimod, deroximel fumarate, ozanimod, |
| | alemtuzumab, mitoxantrone, natalizumab, |
| Neuromyelitis optica spectrum | Corticosteroid, methylprednisolone, azathioprine, |
| disorder | mycophenolate, rituximab, inebilizumab, satralizumab, |
| | eculizumab, |
| Autoimmune encephalitis | Drospirenone, estetrol, steroids, solumedrol, rituximab, |
| | cyclophosphamide, immunoglobulins |
| Infectious meningoencephalitis | Antibiotics, cephalosporins, gentamicin, vancomycin, |
| | cefotxamine, ceftriaxone, ampicillin, antiviral, acyclovir, |
| | corticosteroids, |
| Progressive multifocal | Hexadecyloxypropyl-Cidofovir |
| leukoencephalopathy | |
| paraneoplastic encephalitis | Corticosteroids, cyclophosphamide, invtravenous |
| | immunoglobulin, rituximab, azathioprine, mycophenolate |
| Alzheimer's disease | Galantamine, rivastigmine, donepezil, aducanumab |
| Lewy-Body dementia | Galantamine, rivastigmine, donepezil, caridopa, levodopa, |
| Frontemporal dementia | SSRIs, citalopram, paroxetine, sertraline, trazodone, |
| | olanzapine, quetiapine |
| Encephalopathy | Lactulose, xifaxan, rifaximin, neomycin, gnerlac, enulose, |
| | kristalose, cholac, constilac, |
| Vascular Dementia | Atenolol, metformin, aspirin, memantine, galantamine, |
| | denopezil, rivastigmine, hydergine, nicergoline, nimodipine, |
| Schizophrenia | Olanzapine, aripiprazole, paliperidone, blonanserin, |
| | risperidone, quetiapine, haloperidol, flupenthixol, |
| | zuclopenthixol, clozapine, prochlorperazine, ziprasidone, |
| | brexpiprazole, cariprazine, perphenzaine, chlorpromazine, |
| | asenapine, fluphenazine, loxapine, thiothixene, lurasidone |
| Major Depressive Disorder | SSRIs (e.g., citalopram, escitalopram, paroxetine, fluoxetine, |
| | sertraline), SMSs (e.g., vilazodone, vortioxetine), SNRIs |
| | (e.g, venlafaxine, desvenlafaxine, duloxetine, |
| | levomilnacirpan), TCAs (e.g., amitriptyline, desipramine, |
| | doxepin, imipramine, nortriptyline, protriptyline, |
| | trimipramine), MAOIs, (e.g., phenelzine, tranylcypromine, |
| | isocarboxazid, selegiline), bupropion, esketamine, |
| | mirtazatpin, trazodone |
| Attention-Deficit/Hyperactivity | Amphetamine, methylphenidate, dexmethylphenidate, |
| Disorder | atomoxetine, lisdexamfetamine dimesylate, |
| | dextroamphetamine |
| Bipolar disorder | Mood stabilizers (e.g, lithium, valproic acid, divalproex, |
| | carbamazepine, lamotrigine), antipyschotics (e.g., |
| | olanzapine, risperidone, quetiapine, aripiprazole, |
| | ziprasidone, lurasidone, asenapine), benzodiazepines, or |
| | antidepressants (e.g, SSRIs (e.g., citalopram, escitalopram, |
| | paroxetine, fluoxetine, sertraline), SMSs (e.g., vilazodone, |
| | vortioxetine), SNRIs (e.g, venlafaxine, desvenlafaxine, |
| | duloxetine, levomilnacirpan), TCAs (e.g., amitriptyline, |
| | desipramine, doxepin, imipramine, nortriptyline, |
| | protriptyline, trimipramine), MAOIs, (e.g., phenelzine, |
| | tranylcypromine, isocarboxazid, selegiline), bupropion, |
| | esketamine, mirtazatpin, trazodone) |
| Parkinson's disease | caridopa, levodopa, dopamine agonists (e.g., pramixpexole, |
| | ropinirole, rotigotine, apomorphine), MAO B inhibitors (e.g., |
| | selegiline, rasagiline, safinamide), COMT inhibitors (e.g., |
| | entacapone, opicapone, tolcapone), anticholinergics (e.g., |
| | benztropine, trihexyphenidyl), amantadine |
| Dystonia | Caridopa, levodopa, anticholinergics (e.g., benztropine, |
| | trihexyphenidyl), tetrabenazine, deutetrabenazine, diazepam, |
| | clonazepam, baclofen |
| Amyotrophic lateral sclerosis (ALS) | riluzole, edaravone, |
| Tic disorders | Fluphenazine, haloperidol, risperidone, pimozide, botulinum |
| | toxin, tetrabenazine, methylphenidate, dextroamphetamine, |
| | amphetamine, clonidine, guanfacine, fluoxetine, topiramate |
| Epilepsy | Phenytoin, phenobarbital, carbamazepine, oxcarbazepine, |
| | gabapentin, pregabalin, lacosamide, vigabatrin, valproic |
| | acid, lamotrigine, topiramate, zonisamide, levetriacetam, |
| | clonazepam, rufinamide |
| Stroke | Alteplase, tenecteplase, |
| Trauma | Anti-seizure medications (e.g., phenytoin, valproate, |
| | gabapentin, topiramae, carbamazepine), NMDA antagonists |
| | (e.g., dextromethorphan), barbiturates (e.g., pentobarbital), |
| | calcium channel blockers (e.g., nimodipine), stimulants (e.g, |
| | methylphenidate, modafinil), dopamine agonists (e.g., |
| | sertraline, citalopram, paroxetine), antipyschotics (e.g., |
| | quetiapine) |
| CDKL5 deficiency disorder | A CDKL5 polypeptide or vector encoding CDKL5; an |
| | anticonvulsant (eg . . . , ganaxolone), |
| X-linked severe paediatric monogenic | Enzyme replacement therapy; Anti-seizure medications (e.g., |
| developmental and epilepsy disorder | phenytoin, valproate, gabapentin, topiramae, carbamazepine) |
| occurring via a loss of function | |
| mutation | |
| Tay Sachs disease | Enzyme replacement therapy; Anti-seizure medications (e.g., |
| | phenytoin, valproate, gabapentin, topiramae, carbamazepine) |
| Pompe disease | Enzyme-replacement therapy |
| Gaucher disease | Enzyme-replacement therapy; migulstat; eliglustat; and |
| | osteoporosis drugs |
| Neimann-Pick disease | Enzyme-replacement therapy |
| Fabry disease | Enzyme-replacement therapy |
| Mucopolysaccharidoses (MPS) | Enzyme-replacement therapy (e.g., laronidase) |
| disease | |

In some embodiments of any of the aspects, the central nervous system therapeutic reagent is less than about 500 kDa in size. In some embodiments of any of the aspects, the central nervous system therapeutic reagent is less than 500 kDa in size. In some embodiments of any of the aspects, the central nervous system therapeutic reagent is less than about 300 kDa in size. In some embodiments of any of the aspects, the central nervous system therapeutic reagent is less than 300 kDa in size. In some embodiments of any of the aspects, the central nervous system therapeutic reagent is less than about 200 kDa in size. In some embodiments of any of the aspects, the central nervous system therapeutic reagent is less than 200 kDa in size. In some embodiments of any of the aspects, the central nervous system therapeutic reagent is less than about 70 kDa in size. In some embodiments of any of the aspects, the central nervous system therapeutic reagent is less than 70 kDa in size. In some embodiments of any of the aspects, the central nervous system therapeutic reagent can be, e.g. an enzyme, a protein, a nucleic acid, an antibody reagent, a sugar, and/or a small molecule.

In some embodiments of any of the aspects, the CNS therapeutic agent is an agent that does not normally cross the BBB. In some embodiments of any of the aspects, the CNS therapeutic agent is an agent that inefficiently crosses the BBB, e.g. a therapeutically effective dose of the agent is unable to cross the BBB when administered systemically. In some embodiments of any of the aspects, the CNS therapeutic agent is an agent that does efficiently cross the BBB, e.g. a therapeutically effective dose of the agent is able to cross the BBB when administered systemically. Administration of an inhibitor of Mfsd2A can increase the permeability of the BBB such that, e.g., a therapeutically effective dose of the CNS therapeutic agent is able to reach the CNS or the necessary dose of the CNS therapeutic agent can be lowered.

In some embodiments of any of the aspects, the central nervous system therapeutic agent is administered at least 10 days after the inhibitor of Mfsd2A is first administered. In some embodiments of any of the aspects, the central nervous system therapeutic agent is administered at least 14 days after the inhibitor of Mfsd2A is first administered.

In some embodiments of any of the aspects, the central nervous system therapeutic agent is administered at least 10 days after administration of the inhibitor of Mfsd2A ceases. In some embodiments of any of the aspects, the central nervous system therapeutic agent is administered at least 14 days after the administration of the inhibitor of Mfsd2A ceases.

In some embodiments, a nucleic acid encoding a polypeptide as described herein (e.g. an Mfsd2A polypeptide), or an inhibitory nucleic acid, is comprised by a vector. In some of the aspects described herein, a nucleic acid sequence encoding a polypeptide, or encoding an inhibitory nucleic acid is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

Viral vector systems which can be utilized in the present invention include, but are not limited to, (a) adenovirus vectors; (b) retrovirus vectors, e.g., lentivirus vectors, murine moloney leukemia virus, etc.; (c) adeno-associated virus vectors; (d) herpes simplex virus vectors; (e) SV40 vectors; (f) polyoma virus vectors; (g) papilloma virus vectors; (h) picornavirus vectors; (i) pox virus vectors such as an orthopox, e.g., vaccinia virus vectors or avipox, e.g., canary pox or fowl pox; and (j) a helper-dependent or gutless adenovirus. Replication-defective viruses can also be advantageous. In some embodiments, the vector is an adeno-associated virus vector.

In some embodiments, a viral vector such as an adeno-associated virus (AAV) vector is used. AAVs, which normally infect mammals, including humans, but are non-pathogenic, have been developed and employed as gene therapy vectors in clinical trials in the United States and Europe (Daya and Berns, Clinical Microbiology Reviews 2008, 21, 583-593). AAV vectors may be prepared using any one of a number of methods available to those of ordinary skill in the art. Exemplary AAV vectors are disclosed in Walsh et al., Proc. Soc. Exp. Biol. Med. 204:289-300 (1993); U.S. Pat. No. 5,436,146 which is incorporated herein by reference; Gao et al., Gene Therapy 2005, 5, 285-297; Vandenberghe et al., Gene Therapy 2009, 16, 311-319; Gao et al., PNAS 2002, 99, 11854-11859; Gao et al., PNAS 2003, 100, 6081-6086; Gao et al., J. of Virology 2004, 78, 6381-6388.

In some embodiments, the vector is an adeno-associated virus (AAV) vector. In some embodiments, the AAV vector is an AAV1, AAV2, AAV4, AAV5, AAV6, AAV7, AAV8, AAV9, AAV9.HR, AAVrh. 10, AAVMYO, or AAV2.5. In some embodiments, the AAV is AAV9.

In some embodiments, the vector is episomal. The use of a suitable episomal vector provides a means of maintaining the nucleotide of interest in the subject in high copy number extra chromosomal DNA thereby eliminating potential effects of chromosomal integration.

As non-limiting examples, in some embodiments, a plasmid expression vector can be used. Plasmid expression vectors include, but are not limited to, pcDNA3.1, pET vectors (Novagen®), pGEX vectors (GE Life Sciences), and pMAL vectors (New England labs. Inc.) for protein expression in E. coli host cell such as BL21, BL21 (DE3) and AD494 (DE3) pLysS, Rosetta (DE3), and Origami (DE3) (NovagenR); the strong CMV promoter-based pcDNA3.1 (Invitrogen™ Inc.) and pCIneo vectors (Promega) for expression in mammalian cell lines such as CHO, COS, HEK-293, Jurkat, and MCF-7; replication incompetent adenoviral vector vectors pAdeno X, pAd5F35, pLP-Adeno-X-CMV (Clontech®), pAd/CMV/V5-DEST, pAd-DEST vector (Invitrogen™ Inc.) for adenovirus-mediated gene transfer and expression in mammalian cells; pLNCX2, pLXSN, and pLAPSN retrovirus vectors for use with the Retro-X™ system from Clontech for retroviral-mediated gene transfer and expression in mammalian cells; pLenti4/V5-DEST™, pLenti6/V5-DEST™, and pLenti6.2/V5-GW/lacZ (INVITROGEN™ Inc.) for lentivirus-mediated gene transfer and expression in mammalian cells; adenovirus-associated virus expression vectors such as pAAV-MCS, pAAV-IRES-hrGFP, and pAAV-RC vector (Stratagene®) for adeno-associated virus-mediated gene transfer and expression in mammalian cells.

A retroviral vector can also be used (see Miller et al., Meth. Enzymol. 217:581-599 (1993)). These retroviral vectors contain the components necessary for the correct packaging of the viral genome and integration into the host cell DNA. In another embodiment, the vector is a pox virus such as a vaccinia virus, for example an attenuated vaccinia such as Modified Virus Ankara (MVA) or NYVAC, an avipox such as fowl pox or canary pox. In another embodiment, lentiviral vectors are used, such as the HIV based vectors described in U.S. Pat. Nos. 6,143,520; 5,665,557; and 5,981,276, which are herein incorporated by reference. The vector may or may not be incorporated into the genome of a cell. The constructs may include viral sequences for transfection, if desired. Alternatively, the vector can be capable of episomal replication, e.g., EPV and EBV vectors.

As used herein, “viral particle” refers to a particle comprising at least one viral capsid polypeptide and a nucleic acid molecule, e.g., a viral genome and/or viral vector. Viral vectors are discussed elsewhere herein.

In some embodiments of any of the aspects, the vector is a CNS endothelial-targeting AAV, e.g., an AAV vector that preferentially binds to and/or enters CNS endothelial cells. Such vectors are known in the art and include PHP.V1, BI-30, BR1, or a variant thereof. The sequences and structures of PHP.V1, BI-30, BR1 and their variants are described in Korbelin et al., EMBO Mol Med (2016) 8:609-25; Kumar et al., Nat Methods (2020) 17:541-550; Krolak et al. Nature Cardiovascular Research (2022) 1:389-400; and U.S. Ser. No. 63/320,293 filed Mar. 16, 2022 and WO 2023/004367 published Jan. 26, 2023; each of which is incorporated by reference herein in its entirety.

In some embodiments of any of the aspects, the agonist or inhibitor is targeted to endothelial cells.

In some embodiments of any of the aspects, the vector is an AAV vector with a capsid V1 sequence comprising NNSTRGG (SEQ ID NO: 20). In some embodiments of any of the aspects, the vector comprises a hepatocyte-specific miR-122 target sequence in the 3′ UTR (WPRE). In some embodiments of any of the aspects, the vector comprises a ubiquitous CAG promoter.

In some embodiments, the agonist or inhibitor described herein (and optionally a CNS therapeutic agent) can be present in a scaffold material. Scaffold materials suitable for use in therapeutic compositions are known in the art and can include, but are not limited to, a nanoparticle; a matrix; a hydrogel; and a biomaterial, biocompatible, and/or biodegradable scaffold material. As used herein, the term “nanoparticle” refers to particles that are on the order of about 10-9 or one billionth of a meter. The term “nanoparticle” includes nanospheres; nanorods; nanoshells; and nanoprisms; and these nanoparticles may be part of a nanonetwork.

The term “nanoparticles” also encompasses liposomes and lipid particles having the size of a nanoparticle. As used herein, the term “matrix” refers to a 3-dimensional structure comprising the components of a composition described herein. Non-limiting examples of matrix structures include foams; hydrogels; electrospun fibers; gels; fiber mats; sponges; 3-dimensional scaffolds; non-woven mats; woven materials; knit materials; fiber bundles; and fibers and other material formats (See, e.g. Rockwood et al. Nature Protocols 2011 6:1612-1631 and US Patent Publications 2011/0167602; 2011/0009960; 2012/0296352; and U.S. Pat. No. 8,172,901; each of which is incorporated by reference herein in its entirety). The structure of the matrix can be selected by one of skill in the art depending upon the intended application of the composition, e.g. electrospun matrices can have greater surface area than foams.

In some embodiments, the scaffold is a hydrogel. As used herein, the term “hydrogel” refers to a three-dimensional polymeric structure that is insoluble in water but which is capable of absorbing and retaining large quantities of water to form a stable, often soft and pliable, structure. In some embodiments, water can penetrate in between the polymer chains of the polymer network, subsequently causing swelling and the formation of a hydrogel. In general, hydrogels are superabsorbent. Hydrogels have many desirable properties for biomedical applications. For example, they can be made nontoxic and compatible with tissue, and they are highly permeable to water, ions, and small molecules. Hydrogels are super-absorbent (they can contain over 99% water) and can be comprised of natural (e.g., silk) or synthetic polymers, e.g., PEG.

As used herein, “biomaterial” refers to a material that is biocompatible and biodegradable. As used herein, the term “biocompatible” refers to substances that are not toxic to cells. In some embodiments, a substance is considered to be “biocompatible” if its addition to cells in vitro results in less than or equal to approximately 20% cell death. In some embodiments, a substance is considered to be “biocompatible” if its addition to cells in vivo does not induce inflammation and/or other adverse effects in vivo. As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

In some embodiments, the methods described herein relate to treating a subject having or diagnosed as having a disease affecting the CNS or the retina, e.g. a neurodegenerative disease or a condition treated by delivering therapeutic agents to the CNS. Subjects having a disease affecting the CNS can be identified by a physician using current methods of diagnosing such conditions. Symptoms and/or complications of such conditions which characterize these conditions and aid in diagnosis are well known in the art and include but are not limited to, lost of neural function (e.g. lack of coordination, lack of sensation, altered behaviors, inflammation of the CNS, headaches, etc). Tests that may aid in a diagnosis of such conditions can include, but are not limited to, CT scan, MRI scan, spinal tap, brain biopsy, electroencephalogram (EEG), lumbar puncture, and/or blood tests. For some conditions, a family history of the condition, or exposure to risk factors for the condition can also aid in determining if a subject is likely to have the condition or in making a diagnosis.

In some embodiments of any of the aspects, the disease affecting the CNS is cancer. As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastases. Cancers which migrate from their original location and seed other vital organs can eventually lead to the death of the subject through the functional deterioration of the affected organs.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

The compositions and methods described herein can be administered to a subject having or diagnosed as having a disease affecting the CNS. In some embodiments, the methods described herein comprise administering an effective amount of compositions described herein, to a subject in order to alleviate a symptom of a disease affecting the CNS. As used herein, “alleviating a symptom” is ameliorating any condition or symptom associated with the disease affecting the CNS. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, injection, or intratumoral administration. Administration can be local or systemic.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount of a composition that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active agent which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for BBB permeability, among others. The dosage can be determined by a physician and adjusted, as necessary, to suit observed effects of the treatment.

Effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for tumor growth and/or size among others. The dosage can be determined by a physician and adjusted, as necessary, to suit observed effects of the treatment.

In some embodiments, the technology described herein relates to a pharmaceutical composition comprising an agonist or inhibitor of Mfsd2A, as described herein, and optionally a pharmaceutically acceptable carrier.

In one aspect, described herein is a pharmaceutical composition comprising an inhibitor of Mfsd2A and a pharmaceutically-acceptable carrier. In some embodiments, the composition can further comprise a central nervous system therapeutic agent. In one aspect, described herein is a combination comprising an inhibitor of Mfsd2A and a central nervous system therapeutic agent.

In one aspect, described herein is a pharmaceutical composition comprising an agonist of Mfsd2A and a pharmaceutically-acceptable carrier.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent.

In some embodiments, the pharmaceutical composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a composition as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The methods described herein can further comprise administering a second agent and/or treatment to the subject, e.g. as part of a combinatorial therapy. Non-limiting examples of a second agent and/or treatment can include CNS therapeutic agents as described herein, agents for the treatment of neurodegenerative diseases, and/or agents to treat symptoms or complications of any of the conditions described herein. Further, the methods of treatment can further include the use of surgical treatments.

In some embodiments of any of the aspects, the methods described herein can further comprise administering a second agent and/or treatment to the subject, e.g. as part of a combinatorial therapy. Non-limiting examples of a second agent and/or treatment can include radiation therapy, surgery, gemcitabine, cipslatin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In addition, the methods of treatment can further include the use of radiation or radiation therapy. Further, the methods of treatment can further include the use of surgical treatments.

The methods described herein can further comprise administering a second agent and/or treatment to the subject, e.g. as part of a combinatorial therapy. By way of non-limiting example, if a subject is to be treated for pain or inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs-such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

In certain embodiments, an effective dose of a composition as described herein can be administered to a patient once. In certain embodiments, an effective dose of a composition can be administered to a patient repeatedly. For systemic administration, subjects can be administered a therapeutic amount of a composition, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active agent. The desired dose or amount of effect can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition can be administered over a period of time, such as over a 5 minute, 10 minute, 15 minute, 20 minute, or 25 minute period.

The dosage ranges for the administration of a composition, according to the methods described herein depend upon, for example, the form of the composition, its potency, and the extent to which symptoms, markers, or indicators of a condition described herein are desired to be reduced, for example the percentage modulation desired for permeability of the BBB. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition in, e.g. the treatment of a condition described herein, or to induce a response as described herein (e.g. modulation of BBB permeability) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. BBB permeability to a detectable agent as described herein. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. BBB permeability, or symptoms of a disease affecting the CNS). It is well within the ability of one skilled in the art to monitor efficacy of administration and/or treatment by measuring any one of such parameters, or any combination of parameters. Efficacy can be assessed in animal models of a condition described herein, for example treatment of a disease affecting the CNS of a mouse, or the mouse embryo model of BBB permeability described herein. When using an experimental animal model, efficacy of treatment is evidenced when a statistically significant change in a marker is observed.

In vitro and animal model assays are provided herein which allow the assessment of a given dose of a composition. The efficacy of a given dosage combination can also be assessed in an animal model, e.g. a mouse model.

In one respect, the present invention relates to the herein described compositions, methods, and respective component(s) thereof, as essential to the technology, yet open to the inclusion of unspecified elements, essential or not (“comprising). In some embodiments of any of the aspects, other elements to be included in the description of the composition, method or respective component thereof are limited to those that do not materially affect the basic and novel characteristic(s) of the technology (e.g., the composition, method, or respective component thereof “consists essentially of” the elements described herein). This applies equally to steps within a described method as well as compositions and components therein. In other embodiments of any of the aspects, the compositions, methods, and respective components thereof, described herein are intended to be exclusive of any element not deemed an essential element to the component, composition or method (e.g., the composition, method, or respective component thereof “consists of” the elements described herein). This applies equally to steps within a described method as well as compositions and components therein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, the term “reducing the concentration” refers to a decrease of at least 5%, and preferably at least 10% or more. When applied to, e.g., a concentration of a cellular lipid or phospholipid such as DHA, as described herein, the reduction is sufficient to permit increased transcytosis as that term is defined herein.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a CNS disease. A subject can be male or female.

A subject can be one who has been previously diagnosed with or identified as suffering from or having a condition in need of treatment (e.g. cancer) or one or more complications related to such a condition, and optionally, have already undergone treatment for a condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing. The terms also refer to fragments or variants of the polypeptide that maintain at least 50% of the activity or effect, e.g. of the full length polypeptide. Conservative substitution variants that maintain the activity of wildtype Mfsd2A will include a conservative substitution as defined herein. The identification of amino acids most likely to be tolerant of conservative substitution while maintaining at least 50% of the activity of the wildtype is guided by, for example, sequence alignment with Mfsd2A homologs or paralogs from other species. Amino acids that are identical between Mfsd2A homologs are less likely to tolerate change, while those showing conservative differences are obviously much more likely to tolerate conservative change in the context of an artificial variant. Similarly, positions with non-conservative differences are less likely to be critical to function and more likely to tolerate conservative substitution in an artificial variant. Variants, fragments, and/or fusion proteins can be tested for activity, for example, by administering the variant to an appropriate animal model of CNS disease as described herein.

One method of identifying amino acid residues which can be substituted is to align, for example, human Mfsd2A to a Mfsd2A homolog from one or more non-human species. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. The variant amino acid or DNA sequence can be at least 90%, at least 95%, at least 96%, at least 97%, at least 98%, at least 99%, or more, identical to a native or reference sequence, e.g. a Mfsd2A sequence provided herein or a nucleic acid encoding one of those amino acid sequences. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp or BLASTn (available on the world wide web at blast.ncbi.nlm.nih.gov), with default parameters set.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

In some embodiments, a polypeptide, e.g., a Mfsd2A polypeptide can comprise one or more amino acid substitutions or modifications. In some embodiments, the substitutions and/or modifications can prevent or reduce proteolytic degradation and/or prolong half-life of the polypeptide in a subject. In some embodiments, a polypeptide can be modified by conjugating or fusing it to other polypeptide or polypeptide domains such as, by way of non-limiting example, transferrin (WO06096515A2), albumin (Yeh et al., 1992), growth hormone (US2003104578AA); cellulose (Levy and Shoseyov, 2002); and/or Fc fragments (Ashkenazi and Chamow, 1997). The references in the foregoing paragraph are incorporated by reference herein in their entireties.

In some embodiments, a polypeptide, e.g., a Mfsd2A polypeptide, as described herein can comprise at least one peptide bond replacement. A Mfsd2A polypeptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In some embodiments, a polypeptide, e.g., a Mfsd2A polypeptide, as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser(S), Thr (T), Cys (C), Tyr (Y), Asn (N), Gln (Q), Asp (D), Glu (E), Lys (K), Arg (R), and His (H). In some embodiments, a Mfsd2A polypeptide as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, a polypeptide, e.g. a Mfsd2A polypeptide, can be modified, e.g. by addition of a moiety to one or more of the amino acids that together comprise the peptide. In some embodiments, a polypeptide as described herein can comprise one or more moiety molecules, e.g. 1 or more moiety molecules per polypeptide, 2 or more moiety molecules per polypeptide, 5 or more moiety molecules per polypeptide, 10 or more moiety molecules per polypeptide or more moiety molecules per polypeptide. In some embodiments, a polypeptide as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties. The half-life of a polypeptide can be increased by the addition of moieties, e.g. PEG, albumin, or other fusion partners (e.g. Fc fragment of an immunoglobin).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established. Alterations of the original amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Khudyakov et al. “Artificial DNA: Methods and Applications” CRC Press, 2002; Braman “In Vitro Mutagenesis Protocols” Springer, 2004; and Rapley “The Nucleic Acid Protocols Handbook” Springer 2000; which are herein incorporated by reference in their entireties. In some embodiments, a polypeptide as described herein can be chemically synthesized and mutations can be incorporated as part of the chemical synthesis process.

As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof; including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding portion thereof, and/or bifunctional hybrid antibodies.

Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

As used herein, the term “CDR” refers to the complementarity determining regions within antibody variable sequences. The exact boundaries of these CDRs have been defined differently according to different systems. CDRs may be defined according to the Kabat system (see Kabat, E. A. et al., 1991, “Sequences of Proteins of Immunological Interest”, 5th edit., NIH Publication no. 91-3242, U.S. Department of Health and Human Services). Other systems may be used to define CDRs, which as the system devised by Chothia et al (see Chothia, C. & Lesk, A. M., 1987, “Canonical structures for the hypervariable regions of immunoglobulins”, J. Mol. Biol., 196, 901-917) and the IMGT system (see Lefranc, M. P., 1997, “Unique database numbering system for immunogenetic analysis”, Immunol. Today, 18, 50). An antibody typically contains 3 heavy chain CDRs and 3 light chain CDRs. The term CDR or CDRs is used here to indicate one or several of these regions. A person skilled in the art is able to readily compare the different systems of nomenclature and determine whether a particular sequence may be defined as a CDR. The methods and compositions used herein may utilize CDRs defined according to any of these systems.

The term “antigen-binding portion” of an antibody refers to one or more portions of an antibody as described herein, said one or more portions still having the binding affinities as defined above herein. Portions of a complete antibody have been shown to be able to carry out the antigen-binding function of an antibody. In accordance with the term “antigen-binding portion” of an antibody, examples of binding portions include (i) an Fab portion, i.e., a monovalent portion composed of the VL, VH, CL and CH1 domains; (ii) an F(ab′)2 portion, i.e., a bivalent portion comprising two Fab portions linked to one another in the hinge region via a disulfide bridge; (iii) an Fd portion composed of the VH and CH1 domains; (iv) an Fv portion composed of the FL and VH domains of a single arm of an antibody; and (v) a dAb portion consisting of a VH domain or of VH, CH1, CH2, DH3, or VH, CH2, CH3 (dAbs, or single domain antibodies, comprising only VL domains have also been shown to specifically bind to target epitopes).

Although the two domains of the Fv portion, namely VL and VH, are encoded by separate genes, they may further be linked to one another using a synthetic linker, e.g., a poly-G4S amino acid sequence (‘G4S’) (SEQ ID NO: 100), and recombinant methods, making it possible to prepare them as a single protein chain in which the VL and VH regions combine in order to form monovalent molecules (known as single chain Fv (ScFv)).

The term “antigen-binding portion” of an antibody is also intended to comprise such single chain antibodies. Other forms of single chain antibodies such as “diabodies” can also be included. Diabodies are bivalent, bispecific antibodies in which VH and VL domains are expressed on a single polypeptide chain, but using a linker which is too short for the two domains being able to combine on the same chain, thereby forcing said domains to pair with complementary domains of a different chain and to form two antigen-binding sites. An immunoglobulin constant domain refers to a heavy or light chain constant domain. Human IgG heavy chain and light chain constant domain amino acid sequences are known in the art.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

Furthermore, an antibody reagent as described herein may be part of a larger immunoadhesion molecule formed by covalent or noncovalent association of said antibody or antibody portion with one or more further proteins or peptides. Relevant to such immunoadhesion molecules are the use of the streptavidin core region in order to prepare a tetrameric scFv molecule and the use of a cystein residue, a marker peptide and a C-terminal polyhistidinyl, e.g., hexahistidinyl tag (‘hexahistidinyl tag’) (SEQ ID NO: 101) in order to produce bivalent and biotinylated scFv molecules.

In some embodiments of any of the aspects, the antibody reagent described herein can be an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, and a functionally active epitope-binding portion thereof.

In some embodiments of any of the aspects, the antibody reagent thereof is a fully human antibody. In some embodiments of any of the aspects, the antibody reagent is a humanized antibody or antibody reagent. In some embodiments of any of the aspects, the antibody reagent is a fully humanized antibody or antibody reagent. In some embodiments of any of the aspects, antibody reagent is a chimeric antibody or antibody reagent. In some embodiments of any of the aspects, the antibody reagent is a recombinant polypeptide.

The term “human antibody” refers to antibodies whose variable and constant regions correspond to or are derived from immunoglobulin sequences of the human germ line, as described, for example, by Kabat et al. (see Kabat, et al. (1991) Sequences of Proteins of Immunological Interest, Fifth Edition, U.S. Department of Health and Human Services, NIH Publication No. 91-3242). However, the human antibodies can contain amino acid residues not encoded by human germ line immunoglobulin sequences (for example mutations which have been introduced by random or site-specific mutagenesis in vitro or by somatic mutation in vivo), for example in the CDRs, and in particular in CDR3. Recombinant human antibodies as described herein have variable regions and may also contain constant regions derived from immunoglobulin sequences of the human germ line (see Kabat, E. A., et al. (1991) Sequences of Proteins of Immunological Interest, Fifth Edition, U.S. Department of Health and Human Services, NIH Publication No. 91-3242).

According to particular embodiments, however, such recombinant human antibodies are subjected to in-vitro mutagenesis (or to a somatic in-vivo mutagenesis, if an animal is used which is transgenic due to human Ig sequences) so that the amino acid sequences of the VH and VL regions of the recombinant antibodies are sequences which although related to or derived from VH and VL sequences of the human germ line, do not naturally exist in vivo within the human antibody germ line repertoire. According to particular embodiments, recombinant antibodies of this kind are the result of selective mutagenesis or back mutation or of both. Preferably, mutagenesis leads to an affinity to the target which is greater, and/or an affinity to non-target structures which is smaller than that of the parent antibody.

Generating a humanized antibody from the sequences and information provided herein can be practiced by those of ordinary skill in the art without undue experimentation. In one approach, there are four general steps employed to humanize a monoclonal antibody, see, e.g., U.S. Pat. Nos. 5,585,089; 6,835,823; 6,824,989. These are: (1) determining the nucleotide and predicted amino acid sequence of the starting antibody light and heavy variable domains; (2) designing the humanized antibody, i.e., deciding which antibody framework region to use during the humanizing process; (3) the actual humanizing methodologies/techniques; and (4) the transfection and expression of the humanized antibody.

Usually the CDR regions in humanized antibodies and human antibody variants are substantially identical, and more usually, identical to the corresponding CDR regions in the mouse or human antibody from which they were derived. In some embodiments of any of the aspects, it is possible to make one or more conservative amino acid substitutions of CDR residues without appreciably affecting the binding affinity of the resulting humanized immunoglobulin or human antibody variant. In some embodiments of any of the aspects, substitutions of CDR regions can enhance binding affinity.

The term “chimeric antibody” refers to antibodies which contain sequences for the variable region of the heavy and light chains from one species and constant region sequences from another species, such as antibodies having murine heavy and light chain variable regions linked to human constant regions. Humanized antibodies have variable region framework residues substantially from a human antibody (termed an acceptor antibody) and complementarity determining regions substantially from a non-human antibody, e.g., a mouse-antibody, (referred to as the donor immunoglobulin).

The constant region(s), if present, are also substantially or entirely from a human immunoglobulin. The human variable domains are usually chosen from human antibodies whose framework sequences exhibit a high degree of sequence identity with the (murine) variable region domains from which the CDRs were derived. The heavy and light chain variable region framework residues can be substantially similar to a region of the same or different human antibody sequences. The human antibody sequences can be the sequences of naturally occurring human antibodies or can be consensus sequences of several human antibodies.

In addition, techniques developed for the production of “chimeric antibodies” by splicing genes from a mouse, or other species, antibody molecule of appropriate antigen specificity together with genes from a human antibody molecule of appropriate biological activity can be used. The variable segments of chimeric antibodies are typically linked to at least a portion of an immunoglobulin constant region (Fc), typically that of a human immunoglobulin. Human constant region DNA sequences can be isolated in accordance with well-known procedures from a variety of human cells, such as immortalized B-cells. The antibody can contain both light chain and heavy chain constant regions. The heavy chain constant region can include CH1, hinge, CH2, CH3, and, sometimes, CH4 regions. For therapeutic purposes, the CH2 domain can be deleted or omitted.

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with antibody reagent as described herein. Such functional activities include inhibiting Mfsd2A and/or permeabilizing the BBB.

Additionally, a polypeptide having functional activity means the polypeptide exhibits activity similar, but not necessarily identical to, an activity of a reference antibody reagent, including mature forms, as measured in a particular assay, such as, for example, a biological assay, with or without dose dependency. In the case where dose dependency does exist, it need not be identical to that of the reference antibody reagent, but rather substantially similar to the dose-dependence in a given activity as compared to the reference antibody reagent as described herein (i.e., the candidate polypeptide will exhibit greater activity, or not more than about 25-fold less, about 10-fold less, or about 3-fold less activity relative to the antibody reagent described herein).

In some embodiments of any of the aspects, the antibody reagents described herein are not naturally-occurring biomolecules. For example, a murine antibody raised against an antigen of human origin would not occur in nature absent human intervention and manipulation, e.g., manufacturing steps carried out by a human. Chimeric antibodies are also not naturally-occurring biomolecules, e.g., in that they comprise sequences obtained from multiple species and assembled into a recombinant molecule. In certain particular embodiments, the human antibody reagents described herein are not naturally-occurring biomolecules, e.g., fully human antibodies directed against a human antigen would be subject to negative selection in nature and are not naturally found in the human body.

In some embodiments of any of the aspects, the antibody reagent is an isolated polypeptide. In some embodiments of any of the aspects, the antibody reagent is a purified polypeptide. In some embodiments of any of the aspects, the antibody reagent is an engineered polypeptide.

In some embodiments of any of the aspects, the antibody reagent or antigen-binding fragment thereof is fully human or fully humanized. In some embodiments of any of the aspects, the antibody reagent or antigen-binding fragment thereof is fully humanized except for the CDR sequences. In some embodiments of any of the aspects, the antibody reagent or antigen-binding fragment is selected from the group consisting of: an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, and a bispecific antibody.

As used herein, an “epitope” can be formed on a polypeptide both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin VH/VL pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics.

“Avidity” is the measure of the strength of binding between an antigen-binding molecule (such as an antibody or antigen-binding portion thereof described herein) and the pertinent antigen. Avidity is related to both the affinity between an antigenic determinant and its antigen binding site on the antigen-binding molecule, and the number of pertinent binding sites present on the antigen-binding molecule. Typically, antigen-binding proteins (such as an antibody or portion of an antibody as described herein) will bind to their cognate or specific antigen with a dissociation constant (KD of 10-5 to 10-12 moles/liter or less, such as 10-7 to 10-12 moles/liter or less, or 10-8 to 10-12 moles/liter (i.e., with an association constant (KA) of 105 to 1012 liter/moles or more, such as 107 to 1012 liter/moles or 108 to 1012 liter/moles). Any KD value greater than 10-4 mol/liter (or any KA value lower than 104 M-1) is generally considered to indicate non-specific binding. The KD for biological interactions which are considered meaningful (e.g., specific) are typically in the range of 10-10 M (0.1 nM) to 10-5 M (10000 nM). The stronger an interaction, the lower is its KD. For example, a binding site on an antibody or portion thereof described herein will bind to the desired antigen with an affinity less than 500 nM, such as less than 200 nM, or less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments of any of the aspects, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

In some embodiments of any of the aspects, a reagent that binds specifically has the ability to bind to a target, such as an antigen present on the cell-surface, with a KD 10-5 M (10000 nM) or less, e.g., 10-6 M, 10-7 M, 10-8 M, 10-9 M, 10-10 M, 10-11 M, 10-12 M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay. A polypeptide specifically bound to a target is not displaced by a non-similar competitor. In certain embodiments, an antibody, antigen-binding portion thereof, or CAR is said to specifically bind an antigen when it preferentially recognizes its target antigen in a complex mixture of proteins and/or macromolecules.

In some embodiments of any of the aspects, an antibody reagent as described herein binds to Mfsd2A with a dissociation constant (KD) of 10-5 M (10000 nM) or less, e.g., 10-6 M, 10-7 M, 10-8 M, 10-9 M, 10-10 M, 10-11 M, 10-12 M, or less. In some embodiments of any of the aspects, an antibody reagent as described herein binds to Mfsd2A with a dissociation constant (KD) of from about 10-5 M to 10-6 M. In some embodiments of any of the aspects, an antibody reagent as described herein binds to Mfsd2A with a dissociation constant (KD) of from about 10-6 M to 10-7 M. In some embodiments of any of the aspects, an antibody reagent as described herein binds to Mfsd2A with a dissociation constant (KD) of from about 10-7 M to 10-8 M. In some embodiments of any of the aspects, an antibody reagent as described herein binds to Mfsd2A with a dissociation constant (KD) of from about 10-8 M to 10-9 M. In some embodiments of any of the aspects, an antibody reagent as described herein binds to Mfsd2A with a dissociation constant (KD) of from about 10-9 M to 10-10 M. In some embodiments of any of the aspects, an antibody reagent as described herein binds to Mfsd2A with a dissociation constant (KD) of from about 10-10 M to 10-11 M. In some embodiments of any of the aspects, an antibody reagent as described herein binds to Mfsd2A with a dissociation constant (KD) of from about 10-11 M to 10-12 M. In some embodiments of any of the aspects, an antibody reagent as described herein binds to Mfsd2A with a dissociation constant (KD) of less than 10-12 M.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The term “agent” refers generally to any entity which is normally not present or not present at the levels being administered to a cell, tissue or subject. An agent can be selected from a group including but not limited to: lipids; phospholipids; lipoproteins; polypeptides; small molecules; polynucleotides and antibodies or antigen-binding fragments thereof. A polynucleotide can be RNA or DNA, and can be single or double stranded, and can be selected from a group including, for example, nucleic acids and nucleic acid analogues that encode a polypeptide. A polypeptide can be, but is not limited to, a naturally-occurring polypeptide, a mutated polypeptide or a fragment thereof that retains the function of interest. Further examples of agents include, but are not limited to a nucleic acid aptamer, peptide-nucleic acid (PNA), locked nucleic acid (LNA), small organic or inorganic molecules; saccharide; oligosaccharides; polysaccharides; biological macromolecules, peptidomimetics; nucleic acid analogs and derivatives; extracts made from biological materials such as bacteria, plants, fungi, or mammalian cells or tissues and naturally occurring or synthetic compositions. In some embodiments of any of the aspects, the agent is any chemical, entity or moiety, including without limitation synthetic and naturally-occurring non-proteinaceous entities. In certain embodiments the agent is a small molecule having a chemical moiety selected, for example, from unsubstituted or substituted alkyl, aromatic, or heterocyclyl moieties including macrolides, leptomycins and related natural products or analogues thereof. Agents can be known to have a desired activity and/or property, or can be selected from a library of diverse compounds. As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight more than about 50, but less than about 5000 Daltons (5 kD). Preferably the small molecule has a molecular weight of less than 3 kD, still more preferably less than 2 kD, and most preferably less than 1 kD. In some cases it is preferred that a small molecule have a molecular mass equal to or less than 700 Daltons.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, the agonist or inhibitor described herein is exogenous. In some embodiments of any of the aspects, the agonist or inhibitor described herein is ectopic. In some embodiments of any of the aspects, the agonist or inhibitor described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder described herein. The term “treating” includes reducing or alleviating at least one adverse effect or symptom of a condition, disease or disorder described herein. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments of any of the aspects, a treatment involving the administration of an agonist of Mfsd2A is effective if the permeability of the BBB/BRB is decreased, the integrity of the BBB/BRB is increased, a symptom of the disease is decreased, or the size or rate of growth of a cancer is reduced. In some embodiments of any of the aspects, a treatment involving the administration of an inhibitor of Mfsd2A is effective if the permeability of the BBB/BRB is increased, the integrity of the BBB/BRB is decreased, a symptom of the disease is decreased, the patient's response to the CNS therapeutic agent is improved, or the size or rate of growth of a cancer is reduced.

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom, or contribute to faster recovery from the disease or symptom. Accordingly, the methods described herein can be prophylactic relative to worsening of a symptom of a CNS disease, or the spread or rate of spread of a tumor in the CNS. In some embodiments of any of the aspects, prophylactic treatment is not prevention of all symptoms or signs of a disease.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “nanoparticle” refers to particles that are on the order of about 1 to 1,000 nanometers in diameter or width. The term “nanoparticle” includes nanospheres; nanorods; nanoshells; and nanoprisms; these nanoparticles may be part of a nanonetwork. The term “nanoparticles” also encompasses liposomes and lipid particles having the size of a nanoparticle. Exemplary nanoparticles include lipid nanoparticles or ferritin nanoparticles. Lipid nanoparticles can comprise multiple components, including, e.g., ionizable lipids (such as MC3, DLin-MC3-DMA, ALC-0315, or SM-102), pegylated lipids (such as PEG2000-C-DMG, PEG2000-DMG, ALC-0159), phospholipids (such as DSPC), and cholesterol.

Exemplary liposomes can comprise, e.g., DSPC, DPPC, DSPG, Cholesterol, hydrogenated soy phosphatidylcholine, soy phosphatidyl choline, methoxypolyethylene glycol (mPEG-DSPE) phosphatidyl choline (PC), phosphatidyl glycerol (PG), distearoylphosphatidylcholine, and combinations thereof.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

In all embodiments where a sample is obtained or has been obtained or provided, the sample can be sample taken, obtained, or provided via minimally invasive methods and/or involves only a minor intervention. In some embodiments of any of the aspects, a sample is taken, obtained, or provided by one or more of a blood draw or prick, an epidermal or mucus membrane swab, buccal sampling, saliva sample, a epidermal skin sampling technique, and/or collection of a secreted or expelled bodily fluid (e.g., mucus, urine, sweat, etc), fecal sampling, semen/seminal fluid sampling, or clippings (e.g., of hair or nails). In some embodiments of any of the aspects, the sample comprises, consists of, or consists essentially of blood (or any fraction or component thereof), serum, urine, mucus, epithelial cells, saliva, buccal cells, a secreted or expelled bodily fluid, and/or hair or nail clippings.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:
1. A method of treating a disease selected from the group consisting of:
   a) a primary central nervous cancer or a metastatic disease in the central nervous system;
   b) a neuroinflammatory disorder;
   c) a neurocognitive disorder;
   d) a neurovascular disease; and
   e) a retinal disease;
   in a subject in need thereof, the method comprising administering to the subject an agonist of Mfsd2A.
2. The method of paragraph 1, wherein
   a) the primary central nervous cancer or metastatic disease in the central nervous system is selected from the group consisting of: glioblastoma, CNS lymphoma, meningioma, metastatic melanoma, metastatic breast cancer, metastatic lung cancer, metastatic renal cell cancer, and metastatic colorectal cancer;
   b) the neuroinflammatory disorder is selected from the group consisting of: multiple sclerosis, infectious encephalitis, COVID-19 encephalopathy, sepsis, and CAR-T neurotoxicity (ICANS) and other toxicities from immuno- and cancer therapeutics;
   c) the neurocognitive disorder is selected from the group consisting of: Alzheimer's disease, Huntington disease, Lewy-Body Dementia, and frontotemporal dementia (FTD);
   d) the neurovascular disease is selected from the group consisting of: stroke, trauma, and cerebral edema; and
   e) the retinal disease is selected from a group consisting of: diabetic retinopathy, macular degeneration, macular edema, retinitis pigmentosa, infectious retinitis, and inflammatory retinitis.
3. The method of any of the preceding paragraphs, wherein the disease is glioblastoma.
4. The method of any of the preceding paragraphs, wherein the subject is not further administered a chemotherapeutic.
5. The method of any of the preceding paragraphs, wherein the subject is not further administered a further therapeutic agent.
6. The method of any of the preceding paragraphs, wherein the agonist is a small molecule agonist.
7. The method of any of the preceding paragraphs, wherein the agonist is a pharmaceutical, nutraceutical or dietary formulation, or dietary supplement comprising omega-3 fatty acids and/or linolenic acid.
8. The method of any of the preceding paragraphs, wherein the agonist is a cholesterol lowering drug.
9. The method of any of the preceding paragraphs, wherein the agonist is a Mfsd2A polypeptide or a vector encoding a Mfsd2A polypeptide.
10. The method of paragraph 8, wherein the vector is a CNS endothelial-targeting AAV.
11. The method of paragraph 9, wherein the CNS endothelial-targeting AAV is PHP.V1, BI-30, BR1, or a variant thereof.
12. The method of any of the preceding paragraphs, wherein the agonist is targeted to endothelial cells.
13. A method of treating a disease selected from the group consisting of:
   a) a primary central nervous cancer or a metastatic disease in the central nervous system;
   b) a neuroinflammatory disease;
   c) a neurocognitive disorder;
   d) a neuropsychiatric condition;
   e) a movement disorder;
   f) a critical illness;
   g) an enzyme deficiency disorder; and
   h) a lysosomal storage disorder.
   in a subject in need thereof, the method comprising administering to the subject:
   i) an inhibitor of Mfsd2A; and
   ii) a central nervous system therapeutic agent.
14. The method of paragraph 13, wherein:
   a) the primary central nervous cancer or metastatic disease in the central nervous system is selected from the group consisting of: glioblastoma, CNS lymphoma, meningioma, metastatic melanoma, metastatic breast cancer, metastatic lung cancer, metastatic renal cell cancer, and metastatic colorectal cancer;
   b) the neuroinflammatory disease is selected from the group consisting of: Multiple Sclerosis (MS), neuromyelitis optica (NMO) Spectrum Disorder, autoimmune encephalitis, infectious meningoencephalitis, progressive multifocal leukoencephalopathy (PML), and paraneoplastic encephalitis;
   c) the neurocognitive disorder is selected from the group consisting of: Alzheimer's Dementia, Lewy-Body Dementia, Frontotemporal Dementia, Encephalopathy, and Vascular Demential;
   d) the neuropsychiatric condition is selected from the group consisting of: Schizophrenia, Major Depressive Disorder, Attention-Deficit/Hyperactivity Disorder, and Bipolar Disorder;
   e) the movement disorder is selected from the group consisting of: Parkinson's Disease, Dystonia, ALS, and Tic disorders;
   f) the critical illness is selected from the group consisting of: trauma, stroke, seizure disorders such as epilepsy;
   g) the enzyme deficiency disorder is selected from a lysosomal storage disorder, CDKL5 deficiency disorder, X-linked severe paediatric monogenic developmental and epilepsy disorder occurring via a loss of function mutation; and
   h) the lysosomal storage disorder is selected from the group consisting of: Tay-Sachs disease, Pompe disease, Gaucher disease, Neimann-Pick disease, Fabry disease, and Mucopolysaccharidoses (MPS) disease.
15. The method of any of paragraphs 13-14, wherein the inhibitor is a small molecule inhibitor.
16. The method of any of paragraphs 13-14, wherein the inhibitor is an anti-Mfsd2A antibody reagent, an inhibitory nucleic acid, or a vector encoding an inhibitory nucleic acid.
17. The method of paragraph 16, wherein the inhibitory nucleic acid comprises or consists of the sequence of one or more of SEQ ID NOs: 16-19.
18. The method of any of paragraphs 16-17, wherein the vector is a CNS endothelial-targeting AAV.
19. The method of paragraph 18, wherein the CNS endothelial-targeting AAV is PHP.V1, BI-30, BR1, or a variant thereof.
20. The method of any of the preceding paragraphs, wherein the inhibitor is targeted to endothelial cells.
21. The method of any of paragraphs 13-20, wherein the central nervous system therapeutic agent is administered at least 10 days after the inhibitor of Mfsd2A is first administered.
22. The method of any of paragraphs 13-21, wherein the central nervous system therapeutic agent is administered at least 14 days after the inhibitor of Mfsd2A is first administered.
23. The method of any of paragraphs 13-22, wherein the inhibitor of Mfsd2A is administered for at least 3 months.
24. An agonist of Mfsd2A for use in a method of treating a disease selected from the group consisting of:
   a) a primary central nervous cancer or a metastatic disease in the central nervous system;
   b) a neuroinflammatory disorder;
   c) a neurocognitive disorder;
   d) a neurovascular disease; and
   e) a retinal disease.
25. The agonist of paragraph 24, wherein
   a) the primary central nervous cancer or metastatic disease in the central nervous system is selected from the group consisting of: glioblastoma, CNS lymphoma, meningioma, metastatic melanoma, metastatic breast cancer, metastatic lung cancer, metastatic renal cell cancer, and metastatic colorectal cancer;
   b) the neuroinflammatory disorder is selected from the group consisting of: multiple sclerosis, infectious encephalitis, COVID-19 encephalopathy, sepsis, and CAR-T neurotoxicity (ICANS) and other toxicities from immuno- and cancer therapeutics;
   c) the neurocognitive disorder is selected from the group consisting of: Alzheimer's disease, Huntington disease, Lewy-Body Dementia, and frontotemporal dementia (FTD);
   d) the neurovascular disease is selected from the group consisting of: stroke, trauma, and cerebral edema; and
   e) the retinal disease is selected from a group consisting of: diabetic retinopathy, macular degeneration, macular edema, retinitis pigmentosa, infectious retinitis, and inflammatory retinitis.
26. The agonist of any of paragraphs 24-25, wherein the disease is glioblastoma.
27. The agonist of any of paragraphs 24-26, wherein the subject is not further administered a chemotherapeutic.
28. The agonist of any of paragraphs 24-27, wherein the subject is not further administered a further therapeutic agent.
29. The agonist of any of paragraphs 24-28, wherein the agonist is a small molecule agonist.
30. The agonist of any of paragraphs 24-28, wherein the agonist is a pharmaceutical, nutraceutical or dietary formulation, or dietary supplement comprising omega-3 fatty acids and/or linolenic acid.
31. The agonist of any of paragraphs 24-28, wherein the agonist is a cholesterol lowering drug.
32. The agonist of any of paragraphs 24-28, wherein the agonist is a Mfsd2A polypeptide or a vector encoding a Mfsd2A polypeptide.
33. The agonist of paragraph 32, wherein the vector is a CNS endothelial-targeting AAV.
34. The agonist of paragraph 33 wherein the CNS endothelial-targeting AAV is PHP.V1, BI-30, BR1, or a variant thereof.
35. The agonist of any of paragraphs 24-34, wherein the agonist is targeted to endothelial cells.
36. The combination of:
   i) an inhibitor of Mfsd2A; and
   ii) a central nervous system therapeutic agent;
   for use in a method of treating a disease selected from the group consisting of:
   a) a primary central nervous cancer or a metastatic disease in the central nervous system;
   b) a neuroinflammatory disease;
   c) a neurocognitive disorder;
   d) a neuropsychiatric condition;
   e) a movement disorder;
   f) a critical illness;
   g) an enzyme deficiency disorder; and
   h) a lysosomal storage disorder.
37. The combination of paragraph 36, wherein:
   a) the primary central nervous cancer or metastatic disease in the central nervous system is selected from the group consisting of: glioblastoma, CNS lymphoma, meningioma, metastatic melanoma, metastatic breast cancer, metastatic lung cancer, metastatic renal cell cancer, and metastatic colorectal cancer;
   b) the neuroinflammatory disease is selected from the group consisting of: Multiple Sclerosis (MS), neuromyelitis optica (NMO) Spectrum Disorder, autoimmune encephalitis, infectious meningoencephalitis, progressive multifocal leukoencephalopathy (PML), and paraneoplastic encephalitis;
   c) the neurocognitive disorder is selected from the group consisting of: Alzheimer's Dementia, Lewy-Body Dementia, Frontotemporal Dementia, Encephalopathy, and Vascular Demential;
   d) the neuropsychiatric condition is selected from the group consisting of: Schizophrenia, Major Depressive Disorder, Attention-Deficit/Hyperactivity Disorder, and Bipolar Disorder;
   e) the movement disorder is selected from the group consisting of: Parkinson's Disease, Dystonia, ALS, and Tic disorders;
   f) the critical illness is selected from the group consisting of: trauma, stroke, seizure disorders such as epilepsy;
   g) the enzyme deficiency disorder is selected from a lysosomal storage disorder, CDKL5 deficiency disorder, X-linked severe paediatric monogenic developmental and epilepsy disorder occurring via a loss of function mutation; and
   h) the lysosomal storage disorder is selected from the group consisting of: Tay-Sachs disease, Pompe disease, Gaucher disease, Neimann-Pick disease, Fabry disease, and Mucopolysaccharidoses (MPS) disease.
38. The combination of any of paragraphs 36-37, wherein the inhibitor is a small molecule inhibitor.
39. The combination of any of paragraphs 36-37, wherein the inhibitor is an anti-Mfsd2A antibody reagent, an inhibitory nucleic acid, or a vector encoding an inhibitory nucleic acid.
40. The combination of paragraph 39, wherein the inhibitory nucleic acid comprises or consists of the sequence of one or more of SEQ ID NOs: 16-19.
41. The combination of any of paragraphs 36-37, wherein the vector is a CNS endothelial-targeting AAV.
42. The combination of paragraph 41, wherein the CNS endothelial-targeting AAV is PHP.V1, BI-30, BR1, or a variant thereof.
43. The combination of any of paragraphs 36-42, wherein the inhibitor is targeted to endothelial cells.
44. The combination of any of paragraphs 36-43, wherein the central nervous system therapeutic agent is administered at least 10 days after the inhibitor of Mfsd2A is first administered.
45. The combination of any of paragraphs 36-44, wherein the central nervous system therapeutic agent is administered at least 14 days after the inhibitor of Mfsd2A is first administered.
46. The combination of any of paragraphs 36-45, wherein the inhibitor of Mfsd2A is administered for at least 3 months.

The technology described herein is further illustrated by the following examples which in no way should be construed as being further limiting.

### EXAMPLES

### Example 1

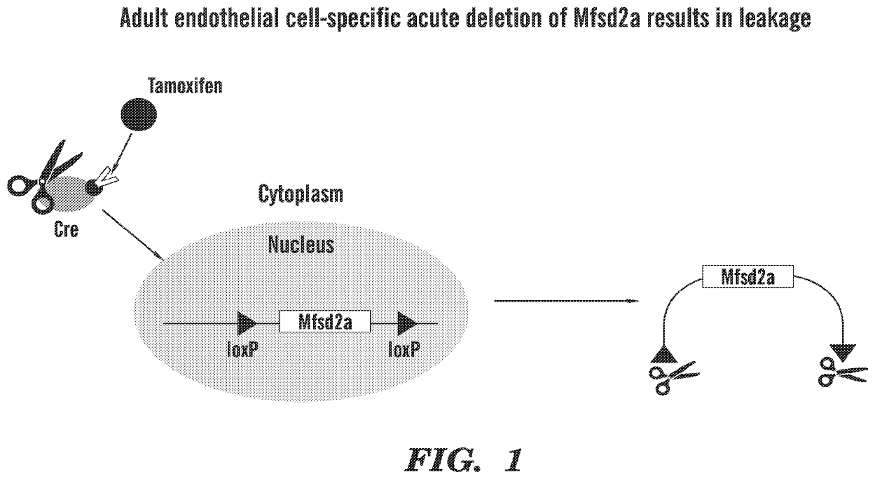

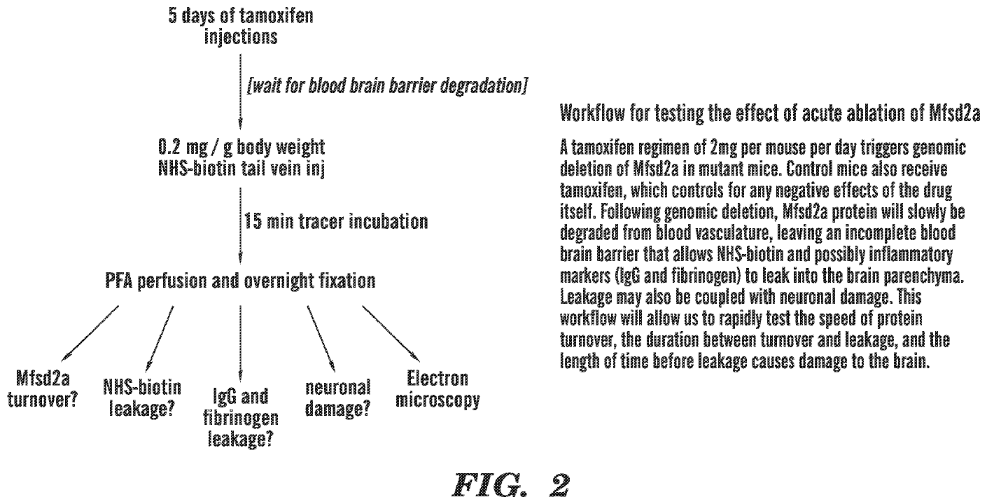

A model permitting endothelial cell-specific deletion of Mfsd2A in adult mice was designed as shown in FIG. 1. The model used a tamoxifen inducible endothelial specific ablation of Mfsd2Aa in adult Mfsd2a f/f/Slcolcl: CreER mice. In mutant mice, Mfsd2a flanked by loxP sites creates a fully functional Mfsd2a protein during development, allowing healthy progression into adulthood. Tamoxifen injected during adulthood binds to the CreER receptor in CNS vasculature and directs the Cre protein into the nuclease. Cre excises at the loxP sites, removing mfsd2a (FIG. 1). In control mice, Mfsd2a is flanked by loxP sites, but there is not Cre, allowing control for tamoxifen. When Mfsd2A is deleted in adult mouse endothelial cells, BBB leakage is observed (FIG. 2).

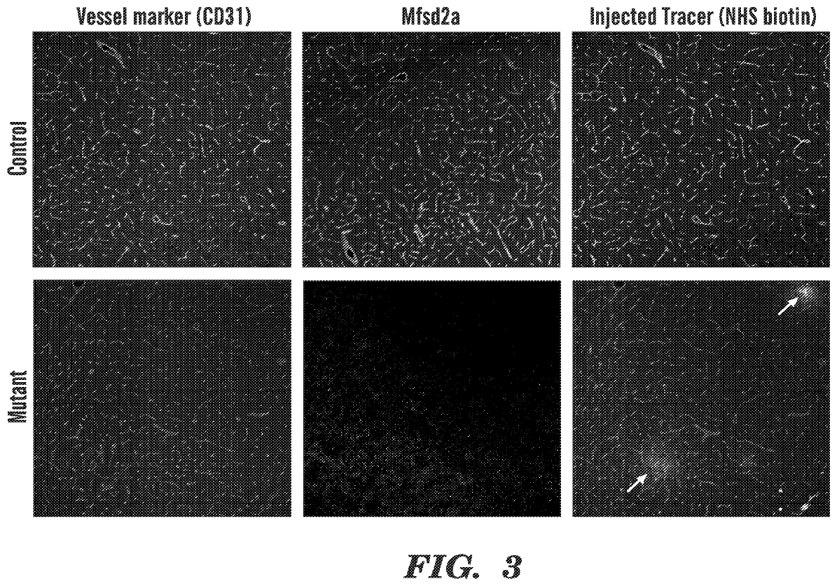

In control (Mfsd2a f/f) mice, the Mfsd2a protein pattern is identical to that of the vessel marker CD31 (FIG. 3). In healthy adult mice, Mfsd2a is only expressed in CNS endothelial cells. Two weeks after tamoxifen injection, the mutant mice (Mfsd2a f/f Slocolcl: CreER+) no longer have Mfsd2a localized to the vascular tree. This demonstrates that two weeks is sufficient for Mfsd2a protein turnover. In control (Mfsd2a f/f) mice, the tail-vein injected tracer (NHS-biotin) is contained within vessels in control mice. In mutant mice the tracer escapes the blood vessels, as marked by the arrows.

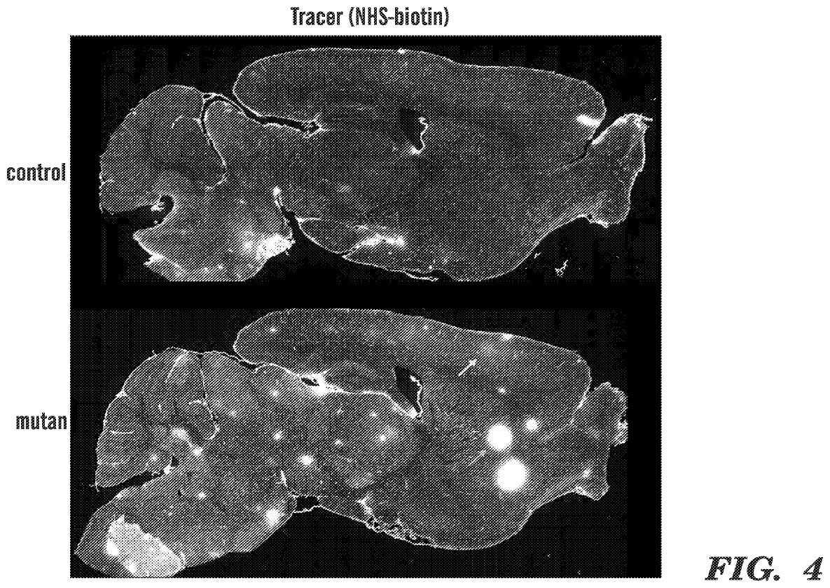

In control mice, the tracer (NHS-biotin) is contained within blood vessels across the entire cross section of the brain (FIG. 4). Mutant mice display leakage throughout the brain, demonstrating the lack of a functional BBB. In many areas (one example marked with an arrow) the leakage forms a circular pattern known as spot leakage. Throughout the cortex there is a more diffuse leakage pattern (example marked by the other arrow).

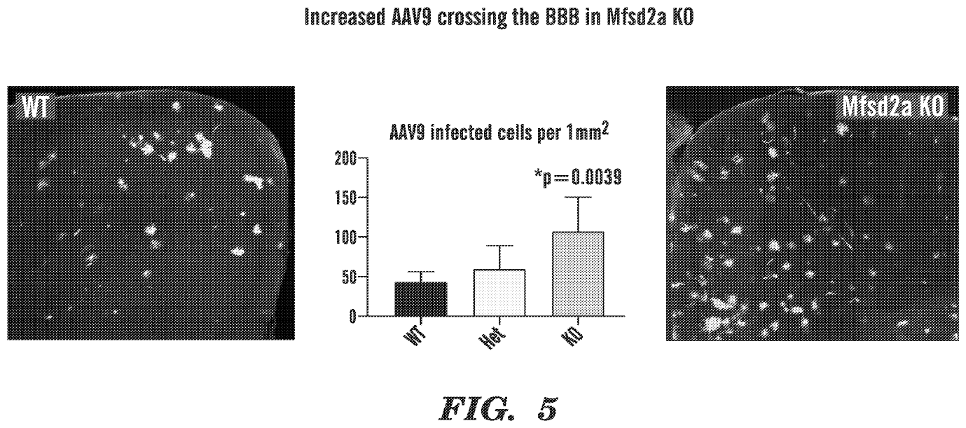

Increased BBB permeability to viral vectors was also demonstrated (FIG. 5).

### Example 2

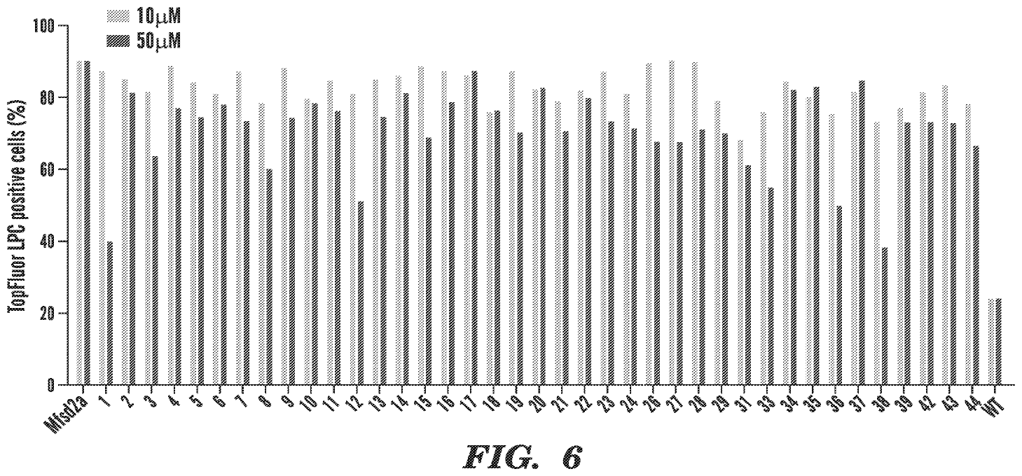

A screen for Mfsd2A inhibitors was conducted. Cells expressing Mfsd2A (control) or not expressing Mfsd2A (WT) were contacting with candidate inhibitors and TopFluor LPC (a fluorescent Mfsd2A substrate). Mfsd2A-expressing cells exhibit significant uptake of the substrate while WT cells exhibit low uptake (FIG. 6). FIG. 6 demonstrates that a number of Mfsd2A inhibitors were identified by the screen.

Sequences

| | | SEQ |
|---|---|---|
| | | ID |
| Sequence | | NO |
| 1 | makgegaesg saagllptsi lqsterpaqv kkepkkkkqq lsvcnklcya lggapyqvtg | 1 |
| 61 | calgfflqiy lldvaqkdee vvfcfssfqv gpfsasiilf vgrawdaitd plvglcisks | |
| 121 | pwtclgrlmp wiifstplav iayfliwfvp dfphgqtywy llfyclfetm vtcfhvpysa | |
| 181 | ltmfisteqt erdsatayrm tvevlgtvlg taiqgqivgq adtpcfqdln sstvasqsan | |
| 241 | hthgttshre tqkayllaag vivciyiica vililgvreq repyeaqqse piayfrglrl | |
| 301 | vmshgpyikl itgflftsla fmlvegnfvl fctytlgfrn efqnlllaim lsatltipiw | |
| 361 | qwfltrfgkk tavyvgissa vpflilvalm esnliityav avaagisvaa afllpwsmlp | |
| 421 | dviddfhlkq phfhgtepif fsfyvfftkf asgvslgist lsldfagyqt rgcsqpervk | |
| 481 | ftlnmlvtma pivlillgll lfkmypidee rrrqnkkalq alrdeasssg csetdstela | |
| 541 | sil | |
| 1 | agagtgcgtt cctcgtctgc cagccggctt ggctagcgcg cggcggccgt ggctaaggct | 2 |
| 61 | gctacgaagc gagcttggga ggagcagcgg cctgcggggc agaggagcat cccgtctacc | |
| 121 | aggtcccaag cggcgtggcc cgcgggtcat ggccaaagga gaaggcgccg agagcggctc | |
| 181 | cgcggcgggg ctgctaccca ccagcatcct ccaaagcact gaacgcccgg cccaggtgaa | |
| 241 | gaaagaaccg aaaaagaaga aacaacagtt gtctgtttgc aacaagcttt gctatgcact | |
| 301 | tgggggagcc ccctaccagg tgacgggctg tgccctgggt ttcttccttc agatctacct | |
| 361 | attggatgtg gctcagaagg atgaggaagt tgtcttttgc ttctcctcat tccaggtggg | |
| 421 | ccctttctct gcctccatca tcctgtttgt gggccgagcc tgggatgcca tcacagaccc | |
| 481 | cctggtgggc ctctgcatca gcaaatcccc ctggacctgc ctgggtcgcc ttatgccctg | |
| 541 | gatcatcttc tccacgcccc tggccgtcat tgcctacttc ctcatctggt tcgtgcccga | |
| 601 | cttcccacac ggccagacct attggtacct gcttttctat tgcctctttg aaacaatggt | |
| 661 | cacgtgtttc catgttccct actcggctct caccatgttc atcagcaccg agcagactga | |
| 721 | gcgggattct gccaccgcct atcggatgac tgtggaagtg ctgggcacag tgctgggcac | |
| 781 | ggcgatccag ggacaaatcg tgggccaagc agacacgcct tgtttccagg acctcaatag | |
| 841 | ctctacagta gcttcacaaa gtgccaacca tacacatggc accacctcac acagggaaac | |
| 901 | gcaaaaggca tacctgctgg cagcgggggt cattgtctgt atctatataa tctgtgctgt | |
| 961 | catcctgatc ctgggcgtgc gggagcagag agaaccctat gaagcccagc agtctgagcc | |
| 1021 | aatcgcctac ttccggggcc tacggctggt catgagccac ggcccataca tcaaacttat | |
| 1081 | tactggcttc ctcttcacct ccttggcttt catgctggtg gaggggaact ttgtcttgtt | |
| 1141 | ttgcacctac accttgggct tccgcaatga attccagaat ctactcctgg ccatcatgct | |
| 1201 | ctcggccact ttaaccattc ccatctggca gtggttcttg acccggtttg gcaagaagac | |
| 1261 | agctgtatat gttgggatct catcagcagt gccatttctc atcttggtgg ccctcatgga | |
| 1321 | gagtaacctc atcattacat atgcggtagc tgtggcagct ggcatcagtg tggcagctgc | |
| 1381 | cttcttacta ccctggtcca tgctgcctga tgtcattgac gacttccatc tgaagcagcc | |
| 1441 | ccacttccat ggaaccgagc ccatcttctt ctccttctat gtcttcttca ccaagtttgc | |
| 1501 | ctctggagtg tcactgggca tttctaccct cagtctggac tttgcagggt accagacccg | |
| 1561 | tggctgctcg cagccggaac gtgtcaagtt tacactgaac atgctcgtga ccatggctcc | |
| 1621 | catagttctc atcctgctgg gcctgctgct cttcaaaatg taccccattg atgaggagag | |
| 1681 | gcggcggcag aataagaagg ccctgcaggc actgagggac gaggccagca gctctggctg | |
| 1741 | ctcagaaaca gactccacag agctggctag catcctctag ggcccgccac gttgcccgaa | |
| 1801 | gccaccatgc agaaggccac agaagggatc aggacctgtc tgccggcttg ctgagcagct | |
| 1861 | ggactgcagg tgctaggaag ggaactgaag actcaaggag gtggcccagg acacttgctg | |
| 1921 | tgctcactgt ggggccggct gctctgtggc ctcctgcctc ccctctgcct gcctgtgggg | |
| 1981 | ccaagccctg gggctgccac tgtgaatatg ccaaggactg atcgggccta gcccggaaca | |
| 2041 | ctaatgtaga aacctttttt tttacagagc ctaattaata acttaatgac tgtgtacata | |
| 2101 | gcaatgtgtg tgtatgtata tgtctgtgag ctattaatgt tattaatttt cataaaagct | |
| 2161 | ggaaagca | |
| MAKGEGAESGSAAGLLPTSILQASERPVQVKKEPKKKQQLSICNKLCYAVGGAPYQLTGC | | 3 |
| ALGFFLQIYLLDVAKVEPLPASIILFVGRAWDAFTDPLVGFCISKSSWTRLGRLMPWIIF | | |
| STPLAIIAYFLIWFVPDFPSGTESSHGFLWYLLFYCLFETLVTCFHVPYSALTMFISTEQ | | |
| SERDSATAYRMTVEVLGTVIGTAIQGQIVGQAKAPCLQDQNGSVVVSEVANRTQSTASLK | | |
| DTQNAYLLAAGIIASIYVLCAFILILGVREQRELYESQQAESMPFFQGLRLVMGHGPYVK | | |
| LIAGFLFTSAFMLVEGNFALFCTYTLDERNEFQNLLLAIMLSATFTIPIWQWELTREGKK | | |
| TAVYIGISSAVPFLILVALMERNLIVTYVVAVAAGVSVAAAFLLPWSMLPDVIDDFHLKH | | |
| PHSPGTEPIFFSFYVFFTKFASGVSLGVSTLSLDFANYQRQGCSQPEQVKFTLKMLVTMA | | |
| PIILILLGLLLFKLYPIDEEKRRQNKKALQALREEASSSGCSDTDSTELASIL | | |
| 1 | mvtcfhvpys altmfisteq terdsatayr mtvevlgtvl gtaiqgqivg qadtpcfqdl | 4 |
| 61 | nsstvasqsa nhthgttshr etqkayllaa gvivciyiic avililgvre qrepyeaqqs | |
| 121 | epiayfrglr lvmshgpyik litgflftsl afmlvegnfv lfctytlgfr nefqnlllai | |
| 181 | mlsatltipi wqwfltrfgk ktavyvgiss avpflilval mesnliitya vavaagisva | |
| 241 | aafllpwsml pdviddfhlk qphfhgtepi ffsfyvfftk fasgvslgis tlsldfagyq | |
| 301 | trgcsqperv kftlnmlvtm apivlillgl llfkmypide errrqnkkal qalrdeasss | |
| 361 | gcsetdstel asil | |
| 1 | makgegaesg saagllptsi lqsterpaqv kiylldvaqv gpfsasiilf vgrawdaitd | 5 |
| 61 | plvglcisks pwtclgrlmp wiifstplav iayfliwfvp dfphgqtywy llfyclfetm | |
| 121 | vtcfhvpysa ltmfisteqt erdsatayrm tvevlgtvlg taiqgqivgq adtpcfqdln | |
| 181 | sstvasqsan hthgttshre tayllaagvi vciyiicavi lilgvreqre pyeaqqsepi | |
| 241 | ayfrglrlvm shgpyiklit gflftslafm lvegnfvlfc tytlgfrnef qnlllaimls | |
| 301 | atltipiwqw fltrfgkkta vyvgissavp flilvalmes nliityavav aagisvaaaf | |
| 361 | llpwsmlpdv iddfhlkqph fhgtepiffs fyvfftkfas gvslgistls ldfagyqtrg | |
| 421 | csqpervkft lnmlvtmapi vlillglllf kmypideerr rqnkkalqal rdeasssgcs | |
| 481 | etdstelasi l | |
| 1 | makgegaesg saagllptsi lqsterpaqk epkkkkqqls vcnklcyalg gapyqvtgca | 6 |
| 61 | lgfflqiyll dvaqvgpfsa siilfvgraw daitdplvgl ciskspwtcl grlmpwiifs | |
| 121 | tplaviayfl iwfvpdfphg qtywyllfyc lfetmvtcfh vpysaltmfi steqterdsa | |
| 181 | tayrmtvevl gtvlgtaiqg qivgqadtpc fqdlnsstva sqsanhthgt tshretqkay | |
| 241 | llaagvivci yiicavilil gvreqrepye aqqsepiayf rglrlvmshg pyiklitgfl | |
| 301 | ftslafmlve gnfvlfctyt lgfrnefqnl llaimlsatl tipiwqwflt rfgkktavyv | |
| 361 | gissavpfli lvalmesnli ityavavaag isvaaafllp wsmlpdvidd fhlkqphfhg | |
| 421 | tepiffsfyv fftkfasgvs lgistlsldf agyqtrgcsq pervkftlnm lvtmapivli | |
| 481 | llglllfkmy pideerrrqn kkalgalrde asssgcsetd stelasil | |
| 1 | makgegaesg saagllptsi lqsterpaqv kkepkkkkqq lsvcnklcya lggapyqvtg | 7 |
| 61 | calgfflqiy lldvaqvgpf sasiilfvgr awdaitdplv glciskspwt clgrlmpwii | |
| 121 | fstplaviay fliwfvpdfp hgqtywyllf yclfetmvtc fhvpysaltm fisteqterd | |
| 181 | satayrmtve vlgtvlgtai qgqivggadt pcfqdlnsst vasqsanhth gttshretqk | |
| 241 | ayllaagviv ciyiicavil ilgvreqrep yeaqqsepia yfrglrlvms hgpyiklitg | |
| 301 | flftslafml vegnfvlfct ytlgfrnefq nlllaimsav pflilvalme snliityava | |
| 361 | vaagisvaaa fllpwsmlpd viddfhlkqp hfhgtepiff sfyvfftkfa sgvslgistl | |
| 421 | sldfagyqtr gcsqpervkf tlnmlvtmap ivlillglll fkmypideer rrqnkkalqa | |
| 481 | lrdeasssgc setdstelas il | |
| 1 | mpwiifstpl aviayfliwf vpdfphgqty wyllfyclfe tmvtcfhvpy saltmfiste | 8 |
| 61 | qterdsatay rmtvevlgtv lgtaiqgqiv gqadtpcfqd lnsstvasqs anhthgttsh | |
| 121 | retqkaylla agvivciyii cavililgvr eqrepyeaqq sepiayfrgl rlvmshgpyi | |
| 181 | klitgflfts lafmlvegnf vlfctytlgf rnefqnllla imlsatltip iwqwfltrfg | |
| 241 | kktavyvgis savpflilva lmesnliity avavaagisv aaafllpwsm lpdviddfhl | |
| 301 | kqphfhgtep iffsfyvfft kfasgvslgi stlsldfagy qtrgcsqper vkftlnmlvt | |
| 361 | mapivlillg lllfkmypid eerrrqnkka lqalrdeass sgcsetdste lasil | |
| 1 | makgegaesg saagllptsi lqsterpaqv kkepkkkkqq lsvcnklcya lggapyqvtg | 9 |
| 61 | calgfflqiy lldvaqvgpf sasiilfvgr awdaitdplv glciskspwt clgrlmpwii | |
| 121 | fstplaviay fliwfvpdfp hgqtywyllf yclfetmvtc fhvpysaltm fisteqterd | |
| 181 | satayrmtve vlgtvlgtai qgqivgqadt pcfqdlnsst vasqsanhth gttshretqk | |
| 241 | ayllaagviv ciyiicavil ilgvreqrep yeaqqsepia yfrglrlvms hgpyiklitg | |
| 301 | flftslafml vegnfvlfct ytlgfrnefq nlllaimlsa tltipiwqwf ltrfgkktav | |
| 361 | yvgissavpf lilvalmesn liityavava agisvaaafl lpwsmlpdvi ddfhlkqphf | |
| 421 | hgtepiffsf yvfftkfasg vslgistlsl dfagyqtrgc sqpervkftl nmlvtmapiv | |
| 481 | lillglllfk mypideerrr qnkkalqalr deasssgcse tdstelasil | |
| 1 | agagtgcgtt cctcgtctgc cagccggctt ggctagcgcg cggcggccgt ggctaaggct | 10 |
| 61 | gctacgaagc gagcttggga ggagcagcgg cctgcggggc agaggagcat cccgtctacc | |
| 121 | aggtcccaag cggcgtggcc cgcgggtcat ggccaaagga gaaggcgccg agagcggctc | |
| 181 | cgcggcgggg ctgctaccca ccagcatcct ccaaagcact gaacgcccgg cccaggtgaa | |
| 241 | ggatcatctt ctccacgccc ctggccgtca ttgcctactt cctcatctgg ttcgtgcccg | |
| 301 | acttcccaca cggccagacc tattggtacc tgcttttcta ttgcctcttt gaaacaatgg | |
| 361 | tcacgtgttt ccatgttccc tactcggctc tcaccatgtt catcagcacc gagcagactg | |
| 421 | agcgggattc tgccaccgcc tatcggatga ctgtggaagt gctgggcaca gtgctgggca | |
| 481 | cggcgatcca gggacaaatc gtgggccaag cagacacgcc ttgtttccag gacctcaata | |
| 541 | gctctacagt agcttcacaa agtgccaacc atacacatgg caccacctca cacagggaaa | |
| 601 | cgcaaaaggc atacctgctg gcagcggggg tcattgtctg tatctatata atctgtgctg | |
| 661 | tcatcctgat cctgggcgtg cgggagcaga gagaacccta tgaagcccag cagtctgagc | |
| 721 | caatcgccta cttccggggc ctacggctgg tcatgagcca cggcccatac atcaaactta | |
| 781 | ttactggctt cctcttcacc tccttggctt tcatgctggt ggaggggaac tttgtcttgt | |
| 841 | tttgcaccta caccttgggc ttccgcaatg aattccagaa tctactcctg gccatcatgc | |
| 901 | tctcggccac tttaaccatt cccatctggc agtggttctt gacccggttt ggcaagaaga | |
| 961 | cagctgtata tgttgggatc tcatcagcag tgccatttct catcttggtg gccctcatgg | |
| 1021 | agagtaacct catcattaca tatgcggtag ctgtggcagc tggcatcagt gtggcagctg | |
| 1081 | ccttcttact accctggtcc atgctgcctg atgtcattga cgacttccat ctgaagcagc | |
| 1141 | cccacttcca tggaaccgag cccatcttct tctccttcta tgtcttcttc accaagtttg | |
| 1201 | cctctggagt gtcactgggc atttctaccc tcagtctgga ctttgcaggg taccagaccc | |
| 1261 | gtggctgctc gcagccggaa cgtgtcaagt ttacactgaa catgctcgtg accatggctc | |
| 1321 | ccatagttct catcctgctg ggcctgctgc tcttcaaaat gtaccccatt gatgaggaga | |
| 1381 | ggcggcggca gaataagaag gccctgcagg cactgaggga cgaggccagc agctctggct | |
| 1441 | gctcagaaac agactccaca gagctggcta gcatcctcta gggcccgcca cgttgcccga | |
| 1501 | agccaccatg cagaaggcca cagaagggat caggacctgt ctgccggctt gctgagcagc | |
| 1561 | tggactgcag gtgctaggaa gggaactgaa gactcaagga ggtggcccag gacacttgct | |
| 1621 | gtgctcactg tggggccggc tgctctgtgg cctcctgcct cccctctgcc tgcctgtggg | |
| 1681 | gccaagccct ggggctgcca ctgtgaatat gccaaggact gatcgggcct agcccggaac | |
| 1741 | actaatgtag aaaccttttt ttttacagag cctaattaat aacttaatga ctgtgtacat | |
| 1801 | agcaatgtgt gtgtatgtat atgtctgtga gctattaatg ttattaattt tcataaaagc | |
| 1861 | tggaaagca | |
| 1 | agagtgcgtt cctcgtctgc cagccggctt ggctagcgcg cggcggccgt ggctaaggct | 11 |
| 61 | gctacgaagc gagcttggga ggagcagcgg cctgcggggc agaggagcat cccgtctacc | |
| 121 | aggtcccaag cggcgtggcc cgcgggtcat ggccaaagga gaaggcgccg agagcggctc | |
| 181 | cgcggcgggg ctgctaccca ccagcatcct ccaaagcact gaacgcccgg cccaggtgaa | |
| 241 | gatctaccta ttggatgtgg ctcaggtggg ccctttctct gcctccatca tcctgtttgt | |
| 301 | gggccgagcc tgggatgcca tcacagaccc cctggtgggc ctctgcatca gcaaatcccc | |
| 361 | ctggacctgc ctgggtcgcc ttatgccctg gatcatcttc tccacgcccc tggccgtcat | |
| 421 | tgcctacttc ctcatctggt tcgtgcccga cttcccacac ggccagacct attggtacct | |
| 481 | gcttttctat tgcctctttg aaacaatggt cacgtgtttc catgttccct actcggctct | |
| 541 | caccatgttc atcagcaccg agcagactga gcgggattct gccaccgcct atcggatgac | |
| 601 | tgtggaagtg ctgggcacag tgctgggcac ggcgatccag ggacaaatcg tgggccaagc | |
| 661 | agacacgcct tgtttccagg acctcaatag ctctacagta gcttcacaaa gtgccaacca | |
| 721 | tacacatggc accacctcac acagggaaac ggcatacctg ctggcagcgg gggtcattgt | |
| 781 | ctgtatctat ataatctgtg ctgtcatcct gatcctgggc gtgcgggagc agagagaacc | |
| 841 | ctatgaagcc cagcagtctg agccaatcgc ctacttccgg ggcctacggc tggtcatgag | |
| 901 | ccacggccca tacatcaaac ttattactgg cttcctcttc acctccttgg ctttcatgct | |
| 961 | ggtggagggg aactttgtct tgttttgcac ctacaccttg ggcttccgca atgaattcca | |
| 1021 | gaatctactc ctggccatca tgctctcggc cactttaacc attcccatct ggcagtggtt | |
| 1081 | cttgacccgg tttggcaaga agacagctgt atatgttggg atctcatcag cagtgccatt | |
| 1141 | tctcatcttg gtggccctca tggagagtaa cctcatcatt acatatgcgg tagctgtggc | |
| 1201 | agctggcatc agtgtggcag ctgccttctt actaccctgg tccatgctgc ctgatgtcat | |
| 1261 | tgacgacttc catctgaagc agccccactt ccatggaacc gagcccatct tcttctcctt | |
| 1321 | ctatgtcttc ttcaccaagt ttgcctctgg agtgtcactg ggcatttcta ccctcagtct | |
| 1381 | ggactttgca gggtaccaga cccgtggctg ctcgcagccg gaacgtgtca agtttacact | |
| 1441 | gaacatgctc gtgaccatgg ctcccatagt tctcatcctg ctgggcctgc tgctcttcaa | |
| 1501 | aatgtacccc attgatgagg agaggcggcg gcagaataag aaggccctgc aggcactgag | |
| 1561 | ggacgaggcc agcagctctg gctgctcaga aacagactcc acagagctgg ctagcatcct | |
| 1621 | ctagggcccg ccacgttgcc cgaagccacc atgcagaagg ccacagaagg gatcaggacc | |
| 1681 | tgtctgccgg cttgctgagc agctggactg caggtgctag gaagggaact gaagactcaa | |
| 1741 | ggaggtggcc caggacactt gctgtgctca ctgtggggcc ggctgctctg tggcctcctg | |
| 1801 | cctcccctct gcctgcctgt ggggccaagc cctggggctg ccactgtgaa tatgccaagg | |
| 1861 | actgatcggg cctagcccgg aacactaatg tagaaacctt tttttttaca gagcctaatt | |
| 1921 | aataacttaa tgactgtgta catagcaatg tgtgtgtatg tatatgtctg tgagctatta | |
| 1981 | atgttattaa ttttcataaa agctggaaag ca | |
| 1 | agagtgcgtt cctcgtctgc cagccggctt ggctagcgcg cggcggccgt ggctaaggct | 12 |
| 61 | gctacgaagc gagcttggga ggagcagcgg cctgcggggc agaggagcat cccgtctacc | |
| 121 | aggtcccaag cggcgtggcc cgcgggtcat ggccaaagga gaaggcgccg agagcggctc | |
| 181 | cgcggcgggg ctgctaccca ccagcatcct ccaaagcact gaacgcccgg cccagaaaga | |
| 241 | accgaaaaag aagaaacaac agttgtctgt ttgcaacaag ctttgctatg cacttggggg | |
| 301 | agccccctac caggtgacgg gctgtgccct gggtttcttc cttcagatct acctattgga | |
| 361 | tgtggctcag gtgggccctt tctctgcctc catcatcctg tttgtgggcc gagcctggga | |
| 421 | tgccatcaca gaccccctgg tgggcctctg catcagcaaa tccccctgga cctgcctggg | |
| 481 | tcgccttatg ccctggatca tcttctccac gcccctggcc gtcattgcct acttcctcat | |
| 541 | ctggttcgtg cccgacttcc cacacggcca gacctattgg tacctgcttt tctattgcct | |
| 601 | ctttgaaaca atggtcacgt gtttccatgt tccctactcg gctctcacca tgttcatcag | |
| 661 | caccgagcag actgagcggg attctgccac cgcctatcgg atgactgtgg aagtgctggg | |
| 721 | cacagtgctg ggcacggcga tccagggaca aatcgtgggc caagcagaca cgccttgttt | |
| 781 | ccaggacctc aatagctcta cagtagcttc acaaagtgcc aaccatacac atggcaccac | |
| 841 | ctcacacagg gaaacgcaaa aggcatacct gctggcagcg ggggtcattg tctgtatcta | |
| 901 | tataatctgt gctgtcatcc tgatcctggg cgtgcgggag cagagagaac cctatgaagc | |
| 961 | ccagcagtct gagccaatcg cctacttccg gggcctacgg ctggtcatga gccacggccc | |
| 1021 | atacatcaaa cttattactg gcttcctctt cacctccttg gctttcatgc tggtggaggg | |
| 1081 | gaactttgtc ttgttttgca cctacacctt gggcttccgc aatgaattcc agaatctact | |
| 1141 | cctggccatc atgctctcgg ccactttaac cattcccatc tggcagtggt tcttgacccg | |
| 1201 | gtttggcaag aagacagctg tatatgttgg gatctcatca gcagtgccat ttctcatctt | |
| 1261 | ggtggccctc atggagagta acctcatcat tacatatgcg gtagctgtgg cagctggcat | |
| 1321 | cagtgtggca gctgccttct tactaccctg gtccatgctg cctgatgtca ttgacgactt | |
| 1381 | ccatctgaag cagccccact tccatggaac cgagcccatc ttcttctcct tctatgtctt | |
| 1441 | cttcaccaag tttgcctctg gagtgtcact gggcatttct accctcagtc tggactttgc | |
| 1501 | agggtaccag acccgtggct gctcgcagcc ggaacgtgtc aagtttacac tgaacatgct | |
| 1561 | cgtgaccatg gctcccatag ttctcatcct gctgggcctg ctgctcttca aaatgtaccc | |
| 1621 | cattgatgag gagaggcggc ggcagaataa gaaggccctg caggcactga gggacgaggc | |
| 1681 | cagcagctct ggctgctcag aaacagactc cacagagctg gctagcatcc tctagggccc | |
| 1741 | gccacgttgc ccgaagccac catgcagaag gccacagaag ggatcaggac ctgtctgccg | |
| 1801 | gcttgctgag cagctggact gcaggtgcta ggaagggaac tgaagactca aggaggtggc | |
| 1861 | ccaggacact tgctgtgctc actgtggggc cggctgctct gtggcctcct gcctcccctc | |
| 1921 | tgcctgcctg tggggccaag ccctggggct gccactgtga atatgccaag gactgatcgg | |
| 1981 | gcctagcccg gaacactaat gtagaaacct ttttttttac agagcctaat taataactta | |
| 2041 | atgactgtgt acatagcaat gtgtgtgtat gtatatgtct gtgagctatt aatgttatta | |
| 2101 | attttcataa aagctggaaa gca | |
| 1 | agagtgcgtt cctcgtctgc cagccggctt ggctagcgcg cggcggccgt ggctaaggct | 13 |
| 61 | gctacgaagc gagcttggga ggagcagcgg cctgcggggc agaggagcat cccgtctacc | |
| 121 | aggtcccaag cggcgtggcc cgcgggtcat ggccaaagga gaaggcgccg agagcggctc | |
| 181 | cgcggcgggg ctgctaccca ccagcatcct ccaaagcact gaacgcccgg cccaggtgaa | |
| 241 | gaaagaaccg aaaaagaaga aacaacagtt gtctgtttgc aacaagcttt gctatgcact | |
| 301 | tgggggagcc ccctaccagg tgacgggctg tgccctgggt ttcttccttc agatctacct | |
| 361 | attggatgtg gctcaggtgg gccctttctc tgcctccatc atcctgtttg tgggccgagc | |
| 421 | ctgggatgcc atcacagacc ccctggtggg cctctgcatc agcaaatccc cctggacctg | |
| 481 | cctgggtcgc cttatgccct ggatcatctt ctccacgccc ctggccgtca ttgcctactt | |
| 541 | cctcatctgg ttcgtgcccg acttcccaca cggccagacc tattggtacc tgcttttcta | |
| 601 | ttgcctcttt gaaacaatgg tcacgtgttt ccatgttccc tactcggctc tcaccatgtt | |
| 661 | catcagcacc gagcagactg agcgggattc tgccaccgcc tatcggatga ctgtggaagt | |
| 721 | gctgggcaca gtgctgggca cggcgatcca gggacaaatc gtgggccaag cagacacgcc | |
| 781 | ttgtttccag gacctcaata gctctacagt agcttcacaa agtgccaacc atacacatgg | |
| 841 | caccacctca cacagggaaa cgcaaaaggc atacctgctg gcagcggggg tcattgtctg | |
| 901 | tatctatata atctgtgctg tcatcctgat cctgggcgtg cgggagcaga gagaacccta | |
| 961 | tgaagcccag cagtctgagc caatcgccta cttccggggc ctacggctgg tcatgagcca | |
| 1021 | cggcccatac atcaaactta ttactggctt cctcttcacc tccttggctt tcatgctggt | |
| 1081 | ggaggggaac tttgtcttgt tttgcaccta caccttgggc ttccgcaatg aattccagaa | |
| 1141 | tctactcctg gccatcatgt cagcagtgcc atttctcatc ttggtggccc tcatggagag | |
| 1201 | taacctcatc attacatatg cggtagctgt ggcagctggc atcagtgtgg cagctgcctt | |
| 1261 | cttactaccc tggtccatgc tgcctgatgt cattgacgac ttccatctga agcagcccca | |
| 1321 | cttccatgga accgagccca tcttcttctc cttctatgtc ttcttcacca agtttgcctc | |
| 1381 | tggagtgtca ctgggcattt ctaccctcag tctggacttt gcagggtacc agacccgtgg | |
| 1441 | ctgctcgcag ccggaacgtg tcaagtttac actgaacatg ctcgtgacca tggctcccat | |
| 1501 | agttctcatc ctgctgggcc tgctgctctt caaaatgtac cccattgatg aggagaggcg | |
| 1561 | gcggcagaat aagaaggccc tgcaggcact gagggacgag gccagcagct ctggctgctc | |
| 1621 | agaaacagac tccacagagc tggctagcat cctctagggc ccgccacgtt gcccgaagcc | |
| 1681 | accatgcaga aggccacaga agggatcagg acctgtctgc cggcttgctg agcagctgga | |
| 1741 | ctgcaggtgc taggaaggga actgaagact caaggaggtg gcccaggaca cttgctgtgc | |
| 1801 | tcactgtggg gccggctgct ctgtggcctc ctgcctcccc tctgcctgcc tgtggggcca | |
| 1861 | agccctgggg ctgccactgt gaatatgcca aggactgatc gggcctagcc cggaacacta | |
| 1921 | atgtagaaac cttttttttt acagagccta attaataact taatgactgt gtacatagca | |
| 1981 | atgtgtgtgt atgtatatgt ctgtgagcta ttaatgttat taattttcat aaaagctgga | |
| 2041 | aagca | |
| 1 | agagtgcgtt cctcgtctgc cagccggctt ggctagcgcg cggcggccgt ggctaaggct | 14 |
| 61 | gctacgaagc gagcttggga ggagcagcgg cctgcggggc agaggagcat cccgtctacc | |
| 121 | aggtcccaag cggcgtggcc cgcgggtcat ggccaaagga gaaggcgccg agagcggctc | |
| 181 | cgcggcgggg ctgctaccca ccagcatcct ccaaagcact gaacgcccgg cccaggtgaa | |
| 241 | gaaccgaaaa agaagaaaca acagttgtct gtttgcaaca agctttgcta tgcacttggg | |
| 301 | ggagccccct accaggtgac gggctgtgcc ctgggtttct tccttcagat ctacctattg | |
| 361 | gatgtggctc aggtgggccc tttctctgcc tccatcatcc tgtttgtggg ccgagcctgg | |
| 421 | gatgccatca cagaccccct ggtgggcctc tgcatcagca aatccccctg gacctgcctg | |
| 481 | ggtcgcctta tgccctggat catcttctcc acgcccctgg ccgtcattgc ctacttcctc | |
| 541 | atctggttcg tgcccgactt cccacacggc cagacctatt ggtacctgct tttctattgc | |
| 601 | ctctttgaaa caatggtcac gtgtttccat gttccctact cggctctcac catgttcatc | |
| 661 | agcaccgagc agactgagcg ggattctgcc accgcctatc ggatgactgt ggaagtgctg | |
| 721 | ggcacagtgc tgggcacggc gatccaggga caaatcgtgg gccaagcaga cacgccttgt | |
| 781 | ttccaggacc tcaatagctc tacagtagct tcacaaagtg ccaaccatac acatggcacc | |
| 841 | acctcacaca gggaaacgca aaaggcatac ctgctggcag cgggggtcat tgtctgtatc | |
| 901 | tatataatct gtgctgtcat cctgatcctg ggcgtgcggg agcagagaga accctatgaa | |
| 961 | gcccagcagt ctgagccaat cgcctacttc cggggcctac ggctggtcat gagccacggc | |
| 1021 | ccatacatca aacttattac tggcttcctc ttcacctcct tggctttcat gctggtggag | |
| 1081 | gggaactttg tcttgttttg cacctacacc ttgggcttcc gcaatgaatt ccagaatcta | |
| 1141 | ctcctggcca tcatgctctc ggccacttta accattccca tctggcagtg gttcttgacc | |
| 1201 | cggtttggca agaagacagc tgtatatgtt gggatctcat cagcagtgcc atttctcatc | |
| 1261 | ttggtggccc tcatggagag taacctcatc attacatatg cggtagctgt ggcagctggc | |
| 1321 | atcagtgtgg cagctgcctt cttactaccc tggtccatgc tgcctgatgt cattgacgac | |
| 1381 | ttccatctga agcagcccca cttccatgga accgagccca tcttcttctc cttctatgtc | |
| 1441 | ttcttcacca agtttgcctc tggagtgtca ctgggcattt ctaccctcag tctggacttt | |
| 1501 | gcagggtacc agacccgtgg ctgctcgcag ccggaacgtg tcaagtttac actgaacatg | |
| 1561 | ctcgtgacca tggctcccat agttctcatc ctgctgggcc tgctgctctt caaaatgtac | |
| 1621 | cccattgatg aggagaggcg gcggcagaat aagaaggccc tgcaggcact gagggacgag | |
| 1681 | gccagcagct ctggctgctc agaaacagac tccacagagc tggctagcat cctctagggc | |
| 1741 | ccgccacgtt gcccgaagcc accatgcaga aggccacaga agggatcagg acctgtctgc | |
| 1801 | cggcttgctg agcagctgga ctgcaggtgc taggaaggga actgaagact caaggaggtg | |
| 1861 | gcccaggaca cttgctgtgc tcactgtggg gccggctgct ctgtggcctc ctgcctcccc | |
| 1921 | tctgcctgcc tgtggggcca agccctgggg ctgccactgt gaatatgcca aggactgatc | |
| 1981 | gggcctagcc cggaacacta atgtagaaac cttttttttt acagagccta attaataact | |
| 2041 | taatgactgt gtacatagca atgtgtgtgt atgtatatgt ctgtgagcta ttaatgttat | |
| 2101 | taattttcat aaaagctgga aagca | |
| 1 | agagtgcgtt cctcgtctgc cagccggctt ggctagcgcg cggcggccgt ggctaaggct | 15 |
| 61 | gctacgaagc gagcttggga ggagcagcgg cctgcggggc agaggagcat cccgtctacc | |
| 121 | aggtcccaag cggcgtggcc cgcgggtcat ggccaaagga gaaggcgccg agagcggctc | |
| 181 | cgcggcgggg ctgctaccca ccagcatcct ccaaagcact gaacgcccgg cccaggtgaa | |
| 241 | gaaagaaccg aaaaagaaga aacaacagtt gtctgtttgc aacaagcttt gctatgcact | |
| 301 | tgggggagcc ccctaccagg tgacgggctg tgccctgggt ttcttccttc agatctacct | |
| 361 | attggatgtg gctcaggtgg gccctttctc tgcctccatc atcctgtttg tgggccgagc | |
| 421 | ctgggatgcc atcacagacc ccctggtggg cctctgcatc agcaaatccc cctggacctg | |
| 481 | cctgggtcgc cttatgccct ggatcatctt ctccacgccc ctggccgtca ttgcctactt | |
| 541 | cctcatctgg ttcgtgcccg acttcccaca cggccagacc tattggtacc tgcttttcta | |
| 601 | ttgcctcttt gaaacaatgg tcacgtgttt ccatgttccc tactcggctc tcaccatgtt | |
| 661 | catcagcacc gagcagactg agcgggattc tgccaccgcc tatcggatga ctgtggaagt | |
| 721 | gctgggcaca gtgctgggca cggcgatcca gggacaaatc gtgggccaag cagacacgcc | |
| 781 | ttgtttccag gacctcaata gctctacagt agcttcacaa agtgccaacc atacacatgg | |
| 841 | caccacctca cacagggaaa cgcaaaaggc atacctgctg gcagcggggg tcattgtctg | |
| 901 | tatctatata atctgtgctg tcatcctgat cctgggcgtg cgggagcaga gagaacccta | |
| 961 | tgaagcccag cagtctgagc caatcgccta cttccggggc ctacggctgg tcatgagcca | |
| 1021 | cggcccatac atcaaactta ttactggctt cctcttcacc tccttggctt tcatgctggt | |
| 1081 | ggaggggaac tttgtcttgt tttgcaccta caccttgggc ttccgcaatg aattccagaa | |
| 1141 | tctactcctg gccatcatgc tctcggccac tttaaccatt cccatctggc agtggttctt | |
| 1201 | gacccggttt ggcaagaaga cagctgtata tgttgggatc tcatcagcag tgccatttct | |
| 1261 | catcttggtg gccctcatgg agagtaacct catcattaca tatgcggtag ctgtggcagc | |
| 1321 | tggcatcagt gtggcagctg ccttcttact accctggtcc atgctgcctg atgtcattga | |
| 1381 | cgacttccat ctgaagcagc cccacttcca tggaaccgag cccatcttct tctccttcta | |
| 1441 | tgtcttcttc accaagtttg cctctggagt gtcactgggc atttctaccc tcagtctgga | |
| 1501 | ctttgcaggg taccagaccc gtggctgctc gcagccggaa cgtgtcaagt ttacactgaa | |
| 1561 | catgctcgtg accatggctc ccatagttct catcctgctg ggcctgctgc tcttcaaaat | |
| 1621 | gtaccccatt gatgaggaga ggcggcggca gaataagaag gccctgcagg cactgaggga | |
| 1681 | cgaggccagc agctctggct gctcagaaac agactccaca gagctggcta gcatcctcta | |
| 1741 | gggcccgcca cgttgcccga agccaccatg cagaaggcca cagaagggat caggacctgt | |
| 1801 | ctgccggctt gctgagcagc tggactgcag gtgctaggaa gggaactgaa gactcaagga | |
| 1861 | ggtggcccag gacacttgct gtgctcactg tggggccggc tgctctgtgg cctcctgcct | |
| 1921 | cccctctgcc tgcctgtggg gccaagccct ggggctgcca ctgtgaatat gccaaggact | |
| 1981 | gatcgggcct agcccggaac actaatgtag aaaccttttt ttttacagag cctaattaat | |
| 2041 | aacttaatga ctgtgtacat agcaatgtgt gtgtatgtat atgtctgtga gctattaatg | |
| p2101 | ttattaattt tcataaaagc tggaaagca | |

1. A method of treating a disease selected from the group consisting of:
a) a primary central nervous cancer or a metastatic disease in the central nervous system;
b) a neuroinflammatory disorder;
c) a neurocognitive disorder;
d) a neurovascular disease; and
e) a retinal disease;
in a subject in need thereof, the method comprising administering to the subject an agonist of Mfsd2A.

2. The method of claim 1, wherein
a) the primary central nervous cancer or metastatic disease in the central nervous system is selected from the group consisting of: glioblastoma, CNS lymphoma, meningioma, metastatic melanoma, metastatic breast cancer, metastatic lung cancer, metastatic renal cell cancer, and metastatic colorectal cancer;
b) the neuroinflammatory disorder is selected from the group consisting of: multiple sclerosis, infectious encephalitis, COVID-19 encephalopathy, sepsis, and CAR-T neurotoxicity (ICANS) and other toxicities from immuno- and cancer therapeutics;
c) the neurocognitive disorder is selected from the group consisting of: Alzheimer's disease, Huntington disease, Lewy-Body Dementia, and frontotemporal dementia (FTD);
d) the neurovascular disease is selected from the group consisting of: stroke, trauma, and cerebral edema; and
e) the retinal disease is selected from a group consisting of: diabetic retinopathy, macular degeneration, macular edema, retinitis pigmentosa, infectious retinitis, and inflammatory retinitis.

3. The method of claim 1, wherein the disease is glioblastoma.

4. The method of claim 1, wherein the subject is not further administered a chemotherapeutic.

5. The method of claim 1, wherein the subject is not further administered a further therapeutic agent.

6. The method of claim 1, wherein the agonist is a small molecule agonist.

7. The method of claim 1, wherein the agonist is a pharmaceutical, nutraceutical or dietary formulation, or dietary supplement comprising omega-3 fatty acids and/or linolenic acid.

8. The method of claim 1, wherein the agonist is a cholesterol lowering drug.

9. The method of claim 1, wherein the agonist is a Mfsd2A polypeptide or a vector encoding a Mfsd2A polypeptide.

10. The method of claim 8, wherein the vector is a CNS endothelial-targeting AAV.

11. The method of claim 9, wherein the CNS endothelial-targeting AAV is PHP.V1, BI-30, BR1, or a variant thereof.

12. The method of claim 1, wherein the agonist is targeted to endothelial cells.

13. A method of treating a disease selected from the group consisting of:
a) a primary central nervous cancer or a metastatic disease in the central nervous system;
b) a neuroinflammatory disease;
c) a neurocognitive disorder;
d) a neuropsychiatric condition;
e) a movement disorder;
f) a critical illness;
g) an enzyme deficiency disorder; and
h) a lysosomal storage disorder.
in a subject in need thereof, the method comprising administering to the subject:
i) an inhibitor of Mfsd2A; and
ii) a central nervous system therapeutic agent.

14. The method of claim 13, wherein:
a) the primary central nervous cancer or metastatic disease in the central nervous system is selected from the group consisting of: glioblastoma, CNS lymphoma, meningioma, metastatic melanoma, metastatic breast cancer, metastatic lung cancer, metastatic renal cell cancer, and metastatic colorectal cancer;
b) the neuroinflammatory disease is selected from the group consisting of: Multiple Sclerosis (MS), neuromyelitis optica (NMO) Spectrum Disorder, autoimmune encephalitis, infectious meningoencephalitis, progressive multifocal leukoencephalopathy (PML), and paraneoplastic encephalitis;
c) the neurocognitive disorder is selected from the group consisting of: Alzheimer's Dementia, Lewy-Body Dementia, Frontotemporal Dementia, Encephalopathy, and Vascular Demential;
d) the neuropsychiatric condition is selected from the group consisting of: Schizophrenia, Major Depressive Disorder, Attention-Deficit/Hyperactivity Disorder, and Bipolar Disorder;
e) the movement disorder is selected from the group consisting of: Parkinson's Disease, Dystonia, ALS, and Tic disorders;
f) the critical illness is selected from the group consisting of: trauma, stroke, seizure disorders such as epilepsy;
g) the enzyme deficiency disorder is selected from a lysosomal storage disorder, CDKL5 deficiency disorder, X-linked severe paediatric monogenic developmental and epilepsy disorder occurring via a loss of function mutation; and
h) the lysosomal storage disorder is selected from the group consisting of: Tay-Sachs disease, Pompe disease, Gaucher disease, Neimann-Pick disease, Fabry disease, and Mucopolysaccharidoses (MPS) disease.

15. The method of claim 13, wherein the inhibitor is a small molecule inhibitor.

16. The method of claim 13, wherein the inhibitor is an anti-Mfsd2A antibody reagent, an inhibitory nucleic acid, or a vector encoding an inhibitory nucleic acid.

17. The method of claim 16, wherein the inhibitory nucleic acid comprises or consists of the sequence of one or more of SEQ ID NOs: 16-19.

18. The method of claim 16, wherein the vector is a CNS endothelial-targeting AAV.

19. (canceled)

20. (canceled)

21. The method of claim 13, wherein the central nervous system therapeutic agent is administered at least 10 days after the inhibitor of Mfsd2A is first administered.

22. (canceled)

23. The method of claim 13, wherein the inhibitor of Mfsd2A is administered for at least 3 months.

24.-46. (canceled)
